(19) United States
(12) Patent Application Publication
(10) Pub. No.: US 2026/0242464 A1
(43) Pub. Date: Aug. 20, 2026

(54) ANTIBODY, NUCLEIC ACID, CELL, AND PHARMACEUTICAL

(71) Applicants: NB HEALTH LABORATORY CO., LTD., Hokkaido (JP); The University of Tokyo, Tokyo (JP)

(72) Inventors: Megumi HIKICHI, Hokkaido (JP); Masami ASHIDA, Hokkaido (JP); Miki NAKAGAWA, Hokkaido (JP); Masahiko SHINAGAWA, Hokkaido (JP); Ryoma TANAKA, Hokkaido (JP); Kiyoshi TAKAYAMA, Hokkaido (JP); Junken AOKI, Tokyo (JP)

(73) Assignees: NB HEALTH LABORATORY CO., LTD., Hokkaido (JP); The University of Tokyo, Tokyo (JP)

(21) Appl. No.: 19/665,742

(22) Filed: May 1, 2026

Related U.S. Application Data
(63) Continuation of application No. 18/881,999, filed on Oct. 13, 2023, now Pat. No. 12,643,946.
(60) Provisional application No. 63/417,197, filed on Oct. 18, 2022.

(30) Foreign Application Priority Data
Apr. 14, 2023 (JP) 2023-066688

(51) Int. Cl. C07K 16/28; A61K 47/68

(52) U.S. Cl. CPC C07K 16/28; A61K 47/6801; C07K 2317/24; C07K 2317/75; C07K 2317/92

(57) ABSTRACT

To provide an antibody that specifically binds to an extracellular domain of human LPA1, wherein the antibody does not specifically bind to an extracellular domain of human LPA2 and does not specifically bind to an extracellular domain of human LPA3. Preferably, the antibody includes an activity of blocking an LPA1-dependent cell function. Also, a nucleic acid encoding the antibody, a cell including the nucleic acid, and a medicament including the antibody as an active ingredient are provided.

### CROSS-REFERENCE TO OTHER APPLICATIONS

This application is a continuation of U.S. application Ser. No. 18/881,999, filed Jan. 7, 2025, which is the National Stage of International Application No. PCT/JP2023/037183, filed Oct. 13, 2023, which claims the benefit of U.S. Provisional Application No. 63/417,197, filed Oct. 18, 2022, and to Japanese Patent Application JP2023-066688, filed Apr. 14, 2023, each of which are herein incorporated by reference in their entirety.

### SEQUENCE LISTING

A sequence listing in electronic (XML file) format is filed with this application and incorporated herein by reference. The name of the XML file is “AttachF_SEQLIST-0356.xml”; the file was created on May 1, 2026; the size of the file is 139,601 bytes.

### TECHNICAL FIELD

This disclosure relates to an antibody that specifically binds to an extracellular domain of human lysophosphatidic acid receptor 1 (human LPA1), a nucleic acid encoding the antibody, a cell including the nucleic acid, and a medicament including the antibody as an active ingredient.

### BACKGROUND ART

Lysophosphatidic acid (LPA) is a lysophospholipid having a glycerol scaffold bonded with one phosphoric acid and one fatty acid. Initially, LPA was thought to be one of intermediate products of lipid metabolites, but it is now recognized as a physiologically active lipid mediator that exhibits a variety of physiological effects. For example, LPA is involved in cell functions such as cell proliferation, apoptosis suppression, cell migration, production of cytokines and chemokines, platelet aggregation, smooth muscle contraction, cell transformation, and neurite regression (Non-Patent Document 1, 2).

LPA binds to a G-protein coupled receptor on a cell surface to modulate the intracellular signaling pathway and exhibit various physiological effects. Six subtypes of LPA receptors, LPA1, LPA2, LPA3, LPA4, LPA5, and LPA6, have been reported (Non-Patent Document 3). Three receptors LPA1, LPA2, and LPA3 belong to Endothelial Differentiation Gene (EDG) family and also referred to as EDG2, EDG4, and EDG7 respectively, which structurally have high homology to each other. LPA4 to LPA6 belong to non-EDG family and have low homology to the EDG family members.

In studies on modulation of LPA receptor functions using cell models and animal models, developmental biological and pathophysiologic influences of the LPA receptor on organs such as nerve, cardiovascular, reproductive organ, lung, liver, and kidney have been investigated. The studies have suggested that LPA-dependent cell dysfunction may be causally involved in diseases including nerve and bone developmental disorder, such as fibrosis, cancer, neuropsychiatric disorder, pain, cardiovascular disease, bone disorder, infertility, and obesity (Non-Patent Documents 1, 2, 4, 5).

Tissue fibrosis is a disease that results from abnormal control of a tissue healing process, leading to tissue dysfunction due to excessive accumulation of extracellular matrix. It has been reported that a LPA concentration in an alveolar lavage fluid in bleomycin-induced fibrosis model mice is increased, and that fibrosis is suppressed in the same models by LPA1 knockout or LPA1 antagonist administration (Non-Patent Documents 6, 7). Also, it has been reported that LPA1 antagonists BMS-986020 and BMS-986278 improve respiratory function and lung fibrosis in patients with idiopathic pulmonary fibrosis (Non-Patent Documents 8, 9). Thus, control of abnormal LPA1-dependent cell functions is useful for treating diseases resulting from fibrosis (e.g., pulmonary fibrosis such as idiopathic pulmonary fibrosis, hepatic fibrosis such as nonalcoholic steatohepatitis, renal fibrosis such as diabetic nephropathy, skin fibrosis such as scleroderma, cardiovascular fibrosis, and gastrointestinal fibrosis) (Non-Patent Documents 1, 2, 10, 11).

Examples of other diseases that have been suggested to be effectively treated by controlling the LPA1-dependent cell functions include neuropathic and inflammatory pain such as fibromyalgia, cancer pain, and diabetic neuropathy (Non-Patent Documents 12 to 14); inflammatory and autoimmune diseases such as rheumatoid arthritis, sepsis, and Guillain-Barre syndrome (Non-Patent Documents 15 to 17); metabolic diseases such as obesity and insulin-resistant diabetes (Non-Patent Document 18); cardiovascular disorders such as atherosclerosis, stroke, and hypertensive nephropathy (Non-Patent Documents 19 to 21); nervous system disorders such as hydrocephalus, schizophrenia, depression, and dementia (Non-Patent Documents 22, 23); cell proliferation diseases (tumor cell proliferation, tumor infiltration and metastasis, and controlled angiogenesis) (Non-Patent Documents 6, 24, 25); urologic disorders such as prostatic hyperplasia and urinary incontinence (Non-Patent Documents 26, 27); and ophthalmological diseases such as ischemic retinopathy (Non-Patent Document 28).

LPA1, LPA2, and LPA3 have different in vivo distributions depending on their subtypes, and each subtype contributes to different cell functions and physiological actions (Non-Patent Documents 1, 2). Thus, a substance that binds specifically to human LPA1 and not specifically to human LPA2 and human LPA3 is desired as a useful active ingredient in pharmaceuticals with low side effects. As reported for the LPA1 antagonist BMS-986020, there is a case where the pharmaceutical binds to multiple LPA receptors, leading to discontinuation of its development due to side effects. It is still not easy to find a substance that binds specifically to human LPA1 and not specifically to human LPA2 and human LPA3.

Although several LPA1 antagonists consisting of low-molecular-weight compounds have been invented, none of them has yet practically utilized (Non-Patent Document 6). Any antibody that binds specifically to LPA1 and not specifically to human LPA2 and LPA3 to control the LPA1-dependent cell functions is useful, but there has been no report on such an antibody.

### PRIOR ART DOCUMENTS

### Non-Patent Documents

Non-Patent Document 1: Aikawa S, Hashimoto T, Kano K, Aoki J. “Lysophosphatidic acid as a lipid mediator with multiple biological actions.” J Biochem. 2015; 157:81-89
Non-Patent Document 2: Yung Y C, Stoddard N C, Chun J. “LPA receptor signaling: pharmacology, physiology, and pathophysiology.” J Lipid Res. 2014; 55: 1192-1214
Non-Patent Document 3: Kihara Y, Maceyka M, Spiegel S, Chun J. “Lysophospholipid receptor nomenclature review: IUPHAR Review 8.” Br J Pharmacol. 2014; 171: 3575-3594
Non-Patent Document 4: Kano K, Aoki J, Hla T. “Lysophospholipid Mediators in Health and Disease.” Annu Rev Pathol. 2022; 17: 459-483
Non-Patent Document 5: Meduri B, Pujar G V, Durai Ananda Kumar T, Akshatha H S, Sethu A K, Singh M, Kanagarla A, Mathew B. “Lysophosphatidic acid (LPA) receptor modulators: Structural features and recent development.” Eur J Med Chem. 2021; 222: 113574
Non-Patent Document 6: Tager A M, LaCamera P, Shea B S, Campanella G S, Selman M, Zhao Z, Polosukhin V, Wain J, Karimi-Shah B A, Kim N D, Hart W K, Pardo A, Blackwell T S, Xu Y, Chun J, Luster A D. “The lysophosphatidic acid receptor LPA11 inks pulmonary fibrosis to lung injury by mediating fibroblast recruitment and vascular leak.” Nat Med. 2008; 14: 45-54
Non-Patent Document 7: Swaney J S, Chapman C, Correa L D, Stebbins K J, Bundey R A, Prodanovich P C, Fagan P, Baccei C S, Santini A M, Hutchinson J H, Seiders T J, Parr T A, Prasit P, Evans J F, Lorrain D S. “A novel, orally active LPA(1) receptor antagonist inhibits lung fibrosis in the mouse bleomycin model.” Br J Pharmacol. 2010; 160: 1699-1713
Non-Patent Document 8: Palmer S M, Snyder L, Todd J L, Soule B, Christian R, Anstrom K, Luo Y, Gagnon R, Rosen G. “Randomized, Double-Blind, Placebo-Controlled, Phase 2 Trial of BMS-986020, a Lysophosphatidic Acid Receptor Antagonist for the Treatment of Idiopathic Pulmonary Fibrosis.” Chest. 2018; 154: 1061-1069
Non-Patent Document 9: Cheng P T W, Kaltenbach R F 3rd, Zhang H, Shi J, Tao S, Li J, Kennedy L J, Walker S J, Shi Y, Wang Y, Dhanusu S, Reddigunta R, Kumaravel S, Jusuf S, Smith D, Krishnananthan S, Li J, Wang T, Heiry R, Sum C S, Kalinowski S S, Hung C P, Chu C H, Azzara A V, Ziegler M, Burns L, Zinker B A, Boehm S, Taylor J, Sapuppo J, Mosure K, Everlof G, Guarino V, Zhang L, Yang Y, Ruan Q, Xu C, Apedo A, Traeger S C, Cvijic M E, Lentz K A, Tirucherai G, Sivaraman L, Robl J, Ellsworth B A, Rosen G, Gordon D A, Soars M G, Gill M, Murphy B J. “Discovery of an Oxycyclohexyl Acid Lysophosphatidic Acid Receptor 1 (LPA1) Antagonist BMS-986278 for the Treatment of Pulmonary Fibrotic Diseases.” J Med Chem. 2021; 64: 15549-15581
Non-Patent Document 10: Kim D, Li H Y, Lee J H, Oh Y S, Jun H S. “Lysophosphatidic acid increases mesangial cell proliferation in models of diabetic nephropathy via Rac1/MAPK/KLF5 signaling.” Exp Mol Med. 2019; 51: 1-10
Non-Patent Document 12: Ueda H. “LPA receptor signaling as a therapeutic target for radical treatment of neuropathic pain and fibromyalgia.” Pain Manag. 2020; 10: 43-53
Non-Patent Document 13: Ueda H, Neyama H, Matsushita Y. “Lysophosphatidic Acid Receptor 1- and 3-Mediated Hyperalgesia and Hypoalgesia in Diabetic Neuropathic Pain Models in Mice.” Cells. 2020; 9: 1906
Non-Patent Document 14: Srikanth M, Chew W S, Hind T, Lim S M, Hay N W J, Lee J H M, Rivera R, Chun J, Ong W Y, Herr D R. “Lysophosphatidic acid and its receptor LPA1 mediate carrageenan induced inflammatory pain in mice.” Eur J Pharmacol. 2018; 841: 49-56
Non-Patent Document 15: Miyabe Y, Miyabe C, Iwai Y, Takayasu A, Fukuda S, Yokoyama W, Nagai J, Jona M, Tokuhara Y, Ohkawa R, Albers H M, Ovaa H, Aoki J, Chun J, Yatomi Y, Ueda H, Miyasaka M, Miyasaka N, Nanki T. “Necessity of lysophosphatidic acid receptor 1 for development of arthritis.” Arthritis Rheum. 2013; 65: 2037-2047
Non-Patent Document 16: Zhao J, Wei J, Weathington N, Jacko A M, Huang H, Tsung A, Zhao Y. “Lysophosphatidic acid receptor 1 antagonist ki16425 blunts abdominal and systemic inflammation in a mouse model of peritoneal sepsis.” Transl Res. 2015; 166: 80-88
Non-Patent Document 17: Szepanowski F, Winkelhausen M, Steubing R D, Mausberg A K, Kleinschnitz C, Stettner M. J “LPA1 signaling drives Schwann cell dedifferentiation in experimental autoimmune neuritis.” Neuroinflammation. 2021; 18: 293
Non-Patent Document 18: D'Souza K, Paramel G V, Kienesberger P C. “Lysophosphatidic Acid Signaling in Obesity and Insulin Resistance.” Nutrients. 2018; 10: 399
Non-Patent Document 19: Zhou Y, Little P J, Ta H T, Xu S, Kamato D. “Lysophosphatidic acid and its receptors: pharmacology and therapeutic potential in atherosclerosis and vascular disease.” Pharmacol Ther. 2019; 204: 107404
Non-Patent Document 20: Gaire B P, Sapkota A, Choi J W. “BMS-986020, a Specific LPA1 Antagonist, Provides Neuroprotection against Ischemic Stroke in Mice.” Antioxidants 2020; 9: 1097
Non-Patent Document 21: Naruse T, Otake H, Takahashi T. “Effects of a lysophosphatidic acid receptor 1 antagonist on hypertensive renal injury in Dahl-Iwai salt-sensitive rats.” J Pharmacol Sci. 2022; 149:179-188
Non-Patent Document 22: Yung Y C, Mutoh T, Lin M E, Noguchi K, Rivera R R, Choi J W, Kingsbury M A, Chun J. “Lysophosphatidic acid signaling may initiate fetal hydrocephalus.” Sci Transl Med. 2011; 3: 99ra87
Non-Patent Document 23: Moreno-Fernandez R D, Tabbai S, Castilla-Ortega E, Perez-Martin M, Estivill-Torrus G, Rodriguez de Fonseca F, Santin L J, Pedraza C. “Stress, Depression, Resilience and Ageing: A Role for the LPA-LPA1 Pathway.” Curr Neuropharmacol. 2018; 16: 271-283
Non-Patent Document 24: Boucharaba A, Serre C M, Guglielmi J, Bordet J C, Clezardin P, Peyruchaud O. “The type 1 lysophosphatidic acid receptor is a target for therapy in bone metastases.” Proc Natl Acad Sci USA. 2006; 103: 9643-9648
Non-Patent Document 25: Zhao P F, Wu S, Li Y, Bao G, Pei J Y, Wang Y W, Ma Q, Sun H J, Damirin A. “LPA receptorl antagonists as anticancer agents suppress human lung tumours.” Eur J Pharmacol. 2020; 868: 172886
Non-Patent Document 26: Sakamoto K, Noguchi Y, Ueshima K, Yamakuni H, Ohtake A, Sato S, Ishizu K, Hosogai N, Kawaminami E, Takeda M, Masuda N. “Effect of ASP6432, a Novel Type 1 Lysophosphatidic Acid Receptor Antagonist, on Urethral Function and Prostate Cell Proliferation. “J Pharmacol Exp Ther. 2018; 366: 390-396
Non-Patent Document 27: Sakamoto K, Noguchi Y, Ueshima K, Ohtake A, Sato S, Imazumi K, Takeda M, Masuda N. “Modulation of urinary frequency via type 1 lysophosphatidic acid receptors: Effect of the novel antagonist ASP6432 in conscious rats.” Eur J Pharmacol. 2019; 853: 11-17
Non-Patent Document 28: Yang C, Lafleur J, Mwaikambo B R, Zhu T, Gagnon C, Chemtob S, Di Polo A, Hardy P. “The role of lysophosphatidic acid receptor (LPA1) in the oxygen-induced retinal ganglion cell degeneration.” Invest Ophthalmol Vis Sci. 2009; 50: 1290-1298

### DISCLOSURE OF INVENTION

### Technical Problem

An object of the present disclosure is to provide a novel antibody that specifically binds to an extracellular domain of LPA1, and a relevant technology.

### Solution to Problem

An aspect of the present disclosure is an antibody that specifically binds to an extracellular domain of human LPA1, wherein the antibody does not specifically bind to an extracellular domain of human LPA2, and the antibody does not specifically bind to an extracellular domain of human LPA3.

Preferably, the antibody includes an activity of blocking an LPA1-dependent cell function.

Preferably, the antibody includes:
a heavy chain CDR1 including an amino acid sequence represented by SEQ ID NO: 1, 11, 21, 31, 41, 51, 61, 71, 81, 91, or 101;
a heavy chain CDR2 including an amino acid sequence represented by SEQ ID NO: 2, 12, 22, 32, 42, 52, 62, 72, 82, 92, or 102;
a heavy chain CDR3 including an amino acid sequence represented by SEQ ID NO: 3, 13, 23, 33, 43, 53, 63, 73, 83, 93, or 103;
a light chain CDR1 including an amino acid sequence represented by SEQ ID NO: 4, 14, 24, 34, 44, 54, 64, 74, 84, 94, or 104;
a light chain CDR2 including an amino acid sequence represented by SEQ ID NO: 5, 15, 25, 35, 45, 55, 65, 75, 85, 95, or 105; and
a light chain CDR3 including an amino acid sequence represented by SEQ ID NO: 6, 16, 26, 36, 46, 56, 66, 76, 86, 96, or 106.

Preferably, the antibody satisfies any one of the following (A1) to (A11):
(A1) including a heavy chain CDR1 including an amino acid sequence represented by SEQ ID NO: 1, a heavy chain CDR2 including an amino acid sequence represented by SEQ ID NO: 2, and a heavy chain CDR3 including an amino acid sequence represented by SEQ ID NO: 3;
(A2) including a heavy chain CDR1 including an amino acid sequence represented by SEQ ID NO: 11, a heavy chain CDR2 including an amino acid sequence represented by SEQ ID NO: 12, and a heavy chain CDR3 including an amino acid sequence represented by SEQ ID NO: 13;
(A3) including a heavy chain CDR1 including an amino acid sequence represented by SEQ ID NO: 21, a heavy chain CDR2 including an amino acid sequence represented by SEQ ID NO: 22, and a heavy chain CDR3 including an amino acid sequence represented by SEQ ID NO: 23;
(A4) including a heavy chain CDR1 including an amino acid sequence represented by SEQ ID NO: 31, a heavy chain CDR2 including an amino acid sequence represented by SEQ ID NO: 32, and a heavy chain CDR3 including an amino acid sequence represented by SEQ ID NO: 33;
(A5) including a heavy chain CDR1 including an amino acid sequence represented by SEQ ID NO: 41, a heavy chain CDR2 including an amino acid sequence represented by SEQ ID NO: 42, and a heavy chain CDR3 including an amino acid sequence represented by SEQ ID NO: 43;
(A6) including a heavy chain CDR1 including an amino acid sequence represented by SEQ ID NO: 51, a heavy chain CDR2 including an amino acid sequence represented by SEQ ID NO: 52, and a heavy chain CDR3 including an amino acid sequence represented by SEQ ID NO: 53;
(A7) including a heavy chain CDR1 including an amino acid sequence represented by SEQ ID NO: 61, a heavy chain CDR2 including an amino acid sequence represented by SEQ ID NO: 62, and a heavy chain CDR3 including an amino acid sequence represented by SEQ ID NO: 63;
(A8) including a heavy chain CDR1 including an amino acid sequence represented by SEQ ID NO: 71, a heavy chain CDR2 including an amino acid sequence represented by SEQ ID NO: 72, and a heavy chain CDR3 including an amino acid sequence represented by SEQ ID NO: 73;
(A9) including a heavy chain CDR1 including an amino acid sequence represented by SEQ ID NO: 81, a heavy chain CDR2 including an amino acid sequence represented by SEQ ID NO: 82, and a heavy chain CDR3 including an amino acid sequence represented by SEQ ID NO: 83;
(A10) including a heavy chain CDR1 including an amino acid sequence represented by SEQ ID NO: 91, a heavy chain CDR2 including an amino acid sequence represented by SEQ ID NO: 92, and a heavy chain CDR3 including an amino acid sequence represented by SEQ ID NO: 93; and
(A11) including a heavy chain CDR1 including an amino acid sequence represented by SEQ ID NO: 101, a heavy chain CDR2 including an amino acid sequence represented by SEQ ID NO: 102, and a heavy chain CDR3 including an amino acid sequence represented by SEQ ID NO: 103.

Preferably, the antibody satisfies any one of the following (B1) to (B11):
(B1) including a light chain CDR1 including an amino acid sequence represented by SEQ ID NO: 4, a light chain CDR2 including an amino acid sequence represented by SEQ ID NO: 5, and a light chain CDR3 including an amino acid sequence represented by SEQ ID NO: 6;
(B2) including a light chain CDR1 including an amino acid sequence represented by SEQ ID NO: 14, a light chain CDR2 including an amino acid sequence represented by SEQ ID NO: 15, and a light chain CDR3 including an amino acid sequence represented by SEQ ID NO: 16;
(B3) including a light chain CDR1 including an amino acid sequence represented by SEQ ID NO: 24, a light chain CDR2 including an amino acid sequence represented by SEQ ID NO: 25, and a light chain CDR3 including an amino acid sequence represented by SEQ ID NO: 26;
(B4) including a light chain CDR1 including an amino acid sequence represented by SEQ ID NO: 34, a light chain CDR2 including an amino acid sequence represented by SEQ ID NO: 35, and a light chain CDR3 including an amino acid sequence represented by SEQ ID NO: 36;
(B5) including a light chain CDR1 including an amino acid sequence represented by SEQ ID NO: 44, a light chain CDR2 including an amino acid sequence represented by SEQ ID NO: 45, and a light chain CDR3 including an amino acid sequence represented by SEQ ID NO: 46;
(B6) including a light chain CDR1 including an amino acid sequence represented by SEQ ID NO: 54, a light chain CDR2 including an amino acid sequence represented by SEQ ID NO: 55, and a light chain CDR3 including an amino acid sequence represented by SEQ ID NO: 56;
(B7) including a light chain CDR1 including an amino acid sequence represented by SEQ ID NO: 64, a light chain CDR2 including an amino acid sequence represented by SEQ ID NO: 65, and a light chain CDR3 including an amino acid sequence represented by SEQ ID NO: 66;
(B8) including a light chain CDR1 including an amino acid sequence represented by SEQ ID NO: 74, a light chain CDR2 including an amino acid sequence represented by SEQ ID NO: 75, and a light chain CDR3 including an amino acid sequence represented by SEQ ID NO: 76;
(B9) including a light chain CDR1 including an amino acid sequence represented by SEQ ID NO: 84, a light chain CDR2 including an amino acid sequence represented by SEQ ID NO: 85, and a light chain CDR3 including an amino acid sequence represented by SEQ ID NO: 86;
(B10) including a light chain CDR1 including an amino acid sequence represented by SEQ ID NO: 94, a light chain CDR2 including an amino acid sequence represented by SEQ ID NO: 95, and a light chain CDR3 including an amino acid sequence represented by SEQ ID NO: 96 and
(B11) including a light chain CDR1 including an amino acid sequence represented by SEQ ID NO: 104, a light chain CDR2 including an amino acid sequence represented by SEQ ID NO: 105, and a light chain CDR3 including an amino acid sequence represented by SEQ ID NO: 106.

Preferably, the antibody satisfies any one of the following (AB1) to (AB11):
(AB1) including a heavy chain CDR1 including an amino acid sequence represented by SEQ ID NO: 1, a heavy chain CDR2 including an amino acid sequence represented by SEQ ID NO: 2, a heavy chain CDR3 including an amino acid sequence represented by SEQ ID NO: 3, a light chain CDR1 including an amino acid sequence represented by SEQ ID NO: 4, a light chain CDR2 including an amino acid sequence represented by SEQ ID NO: 5, and a light chain CDR3 including an amino acid sequence represented by SEQ ID NO: 6;
(AB2) including a heavy chain CDR1 including an amino acid sequence represented by SEQ ID NO: 11, a heavy chain CDR2 including an amino acid sequence represented by SEQ ID NO: 12, a heavy chain CDR3 including an amino acid sequence represented by SEQ ID NO: 13, a light chain CDR1 including an amino acid sequence represented by SEQ ID NO: 14, a light chain CDR2 including an amino acid sequence represented by SEQ ID NO: 15, and a light chain CDR3 including an amino acid sequence represented by SEQ ID NO: 16;
(AB3) including a heavy chain CDR1 including an amino acid sequence represented by SEQ ID NO: 21, a heavy chain CDR2 including an amino acid sequence represented by SEQ ID NO: 22, a heavy chain CDR3 including an amino acid sequence represented by SEQ ID NO: 23, a light chain CDR1 including an amino acid sequence represented by SEQ ID NO: 24, a light chain CDR2 including an amino acid sequence represented by SEQ ID NO: 25, and a light chain CDR3 including an amino acid sequence represented by SEQ ID NO: 26;
(AB4) including a heavy chain CDR1 including an amino acid sequence represented by SEQ ID NO: 31, a heavy chain CDR2 including an amino acid sequence represented by SEQ ID NO: 32, a heavy chain CDR3 including an amino acid sequence represented by SEQ ID NO: 33, a light chain CDR1 including an amino acid sequence represented by SEQ ID NO: 34, a light chain CDR2 including an amino acid sequence represented by SEQ ID NO: 35, and a light chain CDR3 including an amino acid sequence represented by SEQ ID NO: 36;
(AB5) including a heavy chain CDR1 including an amino acid sequence represented by SEQ ID NO: 41, a heavy chain CDR2 including an amino acid sequence represented by SEQ ID NO: 42, a heavy chain CDR3 including an amino acid sequence represented by SEQ ID NO: 43, a light chain CDR1 including an amino acid sequence represented by SEQ ID NO: 44, a light chain CDR2 including an amino acid sequence represented by SEQ ID NO: 45, and a light chain CDR3 including an amino acid sequence represented by SEQ ID NO: 46;
(AB6) including a heavy chain CDR1 including an amino acid sequence represented by SEQ ID NO: 51, a heavy chain CDR2 including an amino acid sequence represented by SEQ ID NO: 52, a heavy chain CDR3 including an amino acid sequence represented by SEQ ID NO: 53, a light chain CDR1 including an amino acid sequence represented by SEQ ID NO: 54, a light chain CDR2 including an amino acid sequence represented by SEQ ID NO: 55, and a light chain CDR3 including an amino acid sequence represented by SEQ ID NO: 56;
(AB7) including a heavy chain CDR1 including an amino acid sequence represented by SEQ ID NO: 61, a heavy chain CDR2 including an amino acid sequence represented by SEQ ID NO: 62, a heavy chain CDR3 including an amino acid sequence represented by SEQ ID NO: 63, a light chain CDR1 including an amino acid sequence represented by SEQ ID NO: 64, a light chain CDR2 including an amino acid sequence represented by SEQ ID NO: 65, and a light chain CDR3 including an amino acid sequence represented by SEQ ID NO: 66;
(AB8) including a heavy chain CDR1 including an amino acid sequence represented by SEQ ID NO: 71, a heavy chain CDR2 including an amino acid sequence represented by SEQ ID NO: 72, a heavy chain CDR3 including an amino acid sequence represented by SEQ ID NO: 73, a light chain CDR1 including an amino acid sequence represented by SEQ ID NO: 74, a light chain CDR2 including an amino acid sequence represented by SEQ ID NO: 75, and a light chain CDR3 including an amino acid sequence represented by SEQ ID NO: 76;
(AB9) including a heavy chain CDR1 including an amino acid sequence represented by SEQ ID NO: 81, a heavy chain CDR2 including an amino acid sequence represented by SEQ ID NO: 82, a heavy chain CDR3 including an amino acid sequence represented by SEQ ID NO: 83, a light chain CDR1 including an amino acid sequence represented by SEQ ID NO: 84, a light chain CDR2 including an amino acid sequence represented by SEQ ID NO: 85, and a light chain CDR3 including an amino acid sequence represented by SEQ ID NO: 86;
(AB10) including a heavy chain CDR1 including an amino acid sequence represented by SEQ ID NO: 91, a heavy chain CDR2 including an amino acid sequence represented by SEQ ID NO: 92, a heavy chain CDR3 including an amino acid sequence represented by SEQ ID NO: 93, a light chain CDR1 including an amino acid sequence represented by SEQ ID NO: 94, a light chain CDR2 including an amino acid sequence represented by SEQ ID NO: 95, and a light chain CDR3 including an amino acid sequence represented by SEQ ID NO: 96; and
(AB11) including a heavy chain CDR1 including an amino acid sequence represented by SEQ ID NO: 101, a heavy chain CDR2 including an amino acid sequence represented by SEQ ID NO: 102, a heavy chain CDR3 including an amino acid sequence represented by SEQ ID NO: 103, a light chain CDR1 including an amino acid sequence represented by SEQ ID NO: 104, a light chain CDR2 including an amino acid sequence represented by SEQ ID NO: 105, and a light chain CDR3 including an amino acid sequence represented by SEQ ID NO: 106.

Preferably, the antibody satisfies any one of the following (C1) to (C11):
(C1) including a heavy chain variable region including an amino acid sequence represented by SEQ ID NO: 7 and a light chain variable region including an amino acid sequence represented by SEQ ID NO: 9;
(C2) including a heavy chain variable region including an amino acid sequence represented by SEQ ID NO: 17 and a light chain variable region including an amino acid sequence represented by SEQ ID NO: 19;
(C3) including a heavy chain variable region including an amino acid sequence represented by SEQ ID NO: 27 and a light chain variable region including an amino acid sequence represented by SEQ ID NO: 29;
(C4) including a heavy chain variable region including an amino acid sequence represented by SEQ ID NO: 37 and a light chain variable region including an amino acid sequence represented by SEQ ID NO: 39;
(C5) including a heavy chain variable region including an amino acid sequence represented by SEQ ID NO: 47 and a light chain variable region including an amino acid sequence represented by SEQ ID NO: 49;
(C6) including a heavy chain variable region including an amino acid sequence represented by SEQ ID NO: 57 and a light chain variable region including an amino acid sequence represented by SEQ ID NO: 59;
(C7) including a heavy chain variable region including an amino acid sequence represented by SEQ ID NO: 67 and a light chain variable region including an amino acid sequence represented by SEQ ID NO: 69;
(C8) including a heavy chain variable region including an amino acid sequence represented by SEQ ID NO: 77 and a light chain variable region including an amino acid sequence represented by SEQ ID NO: 79;
(C9) including a heavy chain variable region including an amino acid sequence represented by SEQ ID NO: 87 and a light chain variable region including an amino acid sequence represented by SEQ ID NO: 89;
(C10) including a heavy chain variable region including an amino acid sequence represented by SEQ ID NO: 97 and a light chain variable region including an amino acid sequence represented by SEQ ID NO: 99; and
(C11) including a heavy chain variable region including an amino acid sequence represented by SEQ ID NO: 107 and a light chain variable region including an amino acid sequence represented by SEQ ID NO: 109.

An aspect of the present disclosure is an antibody that specifically binds to an extracellular domain of human LPA1, wherein
the antibody does not specifically bind to an extracellular domain of human LPA2,
the antibody does not specifically bind to an extracellular domain of human LPA3, and
the antibody satisfies any one of the following (C1′) to (C11′):
(C1′) including a heavy chain variable region including an amino acid sequence represented by SEQ ID NO: 7 with 1 to 10 amino acids substituted, added, or deleted, or including an amino acid sequence having 90% or higher of identity with the amino acid sequence represented by SEQ ID NO: 7, and
a light chain variable region including an amino acid sequence represented by SEQ ID NO: 9 with 1 to 10 amino acids substituted, added, or deleted, or including an amino acid sequence having 90% or higher of identity with the amino acid sequence represented by SEQ ID NO: 9;
(C2′) including a heavy chain variable region including an amino acid sequence represented by SEQ ID NO: 17 with 1 to 10 amino acids substituted, added, or deleted, or including an amino acid sequence having 90% or higher of identity with the amino acid sequence represented by SEQ ID NO: 17, and
a light chain variable region including an amino acid sequence represented by SEQ ID NO: 19 with 1 to 10 amino acids substituted, added, or deleted, or including an amino acid sequence having 90% or higher of identity with the amino acid sequence represented by SEQ ID NO: 19;
(C3′) including a heavy chain variable region including an amino acid sequence represented by SEQ ID NO: 27 with 1 to 10 amino acids substituted, added, or deleted, or including an amino acid sequence having 90% or higher of identity with the amino acid sequence represented by SEQ ID NO: 27, and
a light chain variable region including an amino acid sequence represented by SEQ ID NO: 29 with 1 to 10 amino acids substituted, added, or deleted, or including an amino acid sequence having 90% or higher of identity with the amino acid sequence represented by SEQ ID NO: 29;
(C4′) including a heavy chain variable region including an amino acid sequence represented by SEQ ID NO: 37 with 1 to 10 amino acids substituted, added, or deleted, or including an amino acid sequence having 90% or higher of identity with the amino acid sequence represented by SEQ ID NO: 37, and
a light chain variable region including an amino acid sequence represented by SEQ ID NO: 39 with 1 to 10 amino acids substituted, added, or deleted, or including an amino acid sequence having 90% or higher of identity with the amino acid sequence represented by SEQ ID NO: 39;
(C5′) including a heavy chain variable region including an amino acid sequence represented by SEQ ID NO: 47 with 1 to 10 amino acids substituted, added, or deleted, or including an amino acid sequence having 90% or higher of identity with the amino acid sequence represented by SEQ ID NO: 47, and
a light chain variable region including an amino acid sequence represented by SEQ ID NO: 49 with 1 to 10 amino acids substituted, added, or deleted, or including an amino acid sequence having 90% or higher of identity with the amino acid sequence represented by SEQ ID NO: 49;
(C6′) including a heavy chain variable region including an amino acid sequence represented by SEQ ID NO: 57 with 1 to 10 amino acids substituted, added, or deleted, or including an amino acid sequence having 90% or higher of identity with the amino acid sequence represented by SEQ ID NO: 57, and
a light chain variable region including an amino acid sequence represented by SEQ ID NO: 59 with 1 to 10 amino acids substituted, added, or deleted, or including an amino acid sequence having 90% or higher of identity with the amino acid sequence represented by SEQ ID NO: 59;
(C7′) including a heavy chain variable region including an amino acid sequence represented by SEQ ID NO: 67 with 1 to 10 amino acids substituted, added, or deleted, or including an amino acid sequence having 90% or higher of identity with the amino acid sequence represented by SEQ ID NO: 67, and
a light chain variable region including an amino acid sequence represented by SEQ ID NO: 69 with 1 to 10 amino acids substituted, added, or deleted, or including an amino acid sequence having 90% or higher of identity with the amino acid sequence represented by SEQ ID NO: 69;
(C8′) including a heavy chain variable region including an amino acid sequence represented by SEQ ID NO: 77 with 1 to 10 amino acids substituted, added, or deleted, or including an amino acid sequence having 90% or higher of identity with the amino acid sequence represented by SEQ ID NO: 77, and
a light chain variable region including an amino acid sequence represented by SEQ ID NO: 79 with 1 to 10 amino acids substituted, added, or deleted, or including an amino acid sequence having 90% or higher of identity with the amino acid sequence represented by SEQ ID NO: 79;
(C9′) including a heavy chain variable region including an amino acid sequence represented by SEQ ID NO: 87 with 1 to 10 amino acids substituted, added, or deleted, or including an amino acid sequence having 90% or higher of identity with the amino acid sequence represented by SEQ ID NO: 87, and
a light chain variable region including an amino acid sequence represented by SEQ ID NO: 89 with 1 to 10 amino acids substituted, added, or deleted, or including an amino acid sequence having 90% or higher of identity with the amino acid sequence represented by SEQ ID NO: 89;
(C10′) including a heavy chain variable region including an amino acid sequence represented by SEQ ID NO: 97 with 1 to 10 amino acids substituted, added, or deleted, or including an amino acid sequence having 90% or higher of identity with the amino acid sequence represented by SEQ ID NO: 97, and
a light chain variable region including an amino acid sequence represented by SEQ ID NO: 99 with 1 to 10 amino acids substituted, added, or deleted, or including an amino acid sequence having 90% or higher of identity with the amino acid sequence represented by SEQ ID NO: 99; and
(C11′) including a heavy chain variable region including an amino acid sequence represented by SEQ ID NO: 107 with 1 to 10 amino acids substituted, added, or deleted, or including an amino acid sequence having 90% or higher of identity with the amino acid sequence represented by SEQ ID NO: 107, and
a light chain variable region including an amino acid sequence represented by SEQ ID NO: 109 with 1 to 10 amino acids substituted, added, or deleted, or including an amino acid sequence having 90% or higher of identity with the amino acid sequence represented by SEQ ID NO: 109.

Preferably, the antibody includes an activity of blocking an LPA1-dependent cell function.

An aspect of the present disclosure is a second antibody that specifically binds to an extracellular domain of human LPA1, wherein
the second antibody does not specifically bind to an extracellular domain of human LPA2,
the second antibody does not specifically bind to an extracellular domain of human LPA3, and
the second antibody competitively inhibits a binding between a first antibody that is the above antibody and a receptor.

Preferably, the second antibody includes an activity of blocking an LPA1-dependent cell function.

Preferably, the antibody or the second antibody is a humanized antibody or a chimeric antibody.

Preferably, the antibody or the second antibody is a multispecific antibody.

Preferably, the antibody or the second antibody is a modified antibody bonded with another molecule.

Preferably, the modified antibody is an antibody-drug conjugate.

An aspect of the present disclosure is a nucleic acid encoding the above antibody or second antibody.

An aspect of the present disclosure is a cell including the above nucleic acid.

An aspect of the present disclosure is a medicament including the above antibody as an active ingredient.

Preferably, the medicament is used for treating a disease, a disorder, or a condition involving LPA1-dependent cell dysfunction.

Preferably, the medicament is used for treating tissue fibrosis, cell proliferative disease, pain, inflammatory disease, autoimmune disease, metabolic disease, cardiovascular disorder, urological disease, or ophthalmological disease.

Preferably, the tissue fibrosis is hepatic fibrosis, kidney fibrosis, pulmonary fibrosis, skin fibrosis, cardiovascular fibrosis, or gastrointestinal fibrosis.

Preferably, the hepatic fibrosis is nonalcoholic steatohepatitis (NASH), nonalcoholic fatty liver disease (NAFLD), liver cirrhosis, ischemia reperfusion, post-liver implantation disorder, necrotizing hepatitis, hepatitis B, hepatitis C, primary biliary liver cirrhosis, or primary sclerosing cholangitis.

Preferably, the liver cirrhosis is alcohol-induced, drug-induced, or chemically induced.

Preferably, the kidney fibrosis is proliferative glomerulonephritis, sclerosing glomerulonephritis, nephrogenic fibrosing dermopathy, diabetic nephropathy, renal tubulointerstitial fibrosis, or focal segmental glomerulosclerosis.

Preferably, the pulmonary fibrosis is lung interstitial fibrosis, drug-induced sarcoidosis, idiopathic pulmonary fibrosis, asthma, chronic obstructive lung disease, diffuse alveolar damage disease, pulmonary hypertension, or neonatal bronchopulmonary malformation.

Preferably, the skin fibrosis is scleroderma, keloid scarring, psoriasis, hypertrophic scarring, or pseudo-scleroderma.

Preferably, the cardiovascular fibrosis is atherosclerosis, coronary artery restenosis, congestive cardiomyopathy, heart failure, heart implantation, or myocardial fibrosis.

Preferably, the gastrointestinal fibrosis is collagenous colitis, villous atrophy, crypt hyperplasia, polyp formation, Crohn's fibrosis, gastric ulcer healing, or scar after abdominal adhesion surgery.

Preferably, the fibrosis is caused by a bone-related fiberization disease and has rheumatoid pannus formation.

Preferably, the cell proliferative disease is tumor cell proliferation, tumor infiltration and metastasis, or controlled angiogenesis.

Preferably, the cell proliferative disease is hematological cancer or solid carcinoma.

Preferably, the solid carcinoma is breast cancer, malignant breast tumor, gastric cancer, melanoma, non-small-cell lung cancer, pulmonary adenocarcinoma, gastric cancer, pancreatic cancer, ovarian cancer, uterine cancer, cervical cancer, hepatoma, prostate cancer, urothelial cancer, renal cell cancer, or squamous cell cancer.

Preferably, the squamous cell cancer is oral squamous cell carcinoma, esophageal squamous cell carcinoma, or pharyngeal squamous cell carcinoma.

Preferably, the pain is fibromyalgia pain, cancer pain, or diabetic neuropathy pain.

Preferably, the inflammatory disease is rheumatoid arthritis, sepsis, chronic obstructive lung disease, inflammatory bowel disease, implanted organ rejection, Guillain-Barre syndrome, or multiple sclerosis.

Preferably, the metabolic disease is obesity or insulin-resistant diabetes.

Preferably, the cardiovascular disorder is stroke, hypertensive nephropathy, or Raynaud's phenomenon.

Preferably, the nervous system disorder is hydrocephalus, schizophrenia, depression, or dementia.

Preferably, the urologic disease is prostatic hyperplasia or urinary incontinence.

Preferably, the ophthalmological disease is ischemic retinopathy, diabetic retinopathy, or age-related macular degeneration.

### Effect of Invention

According to the present disclosure, it is possible to provide a novel antibody that specifically binds to an extracellular domain of LPA1, and a relevant technology.

### BRIEF DESCRIPTION OF DRAWINGS

FIG. 1A is a histogram presenting results of flow cytometry performed in Example 4, showing binding properties between an antibody and human LPA1 to LPA3.

FIG. 1B is a histogram presenting results of flow cytometry performed in Example 4, showing binding properties between an antibody and mouse LPA1 to LPA3.

FIG. 2A is a graph presenting an evaluation result of a binding property of an antibody (210309-1-C) to a human LPA1-stably expressing CHO cell.

FIG. 2B is a graph presenting an evaluation result of a binding property of an antibody (210309-1-G) to a human LPA1-stably expressing CHO cell.

FIG. 2C is a graph presenting an evaluation result of a binding property of an antibody (210309-2-A) to a human LPA1-stably expressing CHO cell.

FIG. 2D is a graph presenting an evaluation result of a binding property of an antibody (210309-2-D) to a human LPA1-stably expressing CHO cell.

FIG. 2E is a graph presenting an evaluation result of a binding property of an antibody (210309-4-A) to a human LPA1-stably expressing CHO cell.

FIG. 2F is a graph presenting an evaluation result of a binding property of an antibody (210310-1-D) to a human LPA1-stably expressing CHO cell.

FIG. 2G is a graph presenting an evaluation result of a binding property of an antibody (210420-4-E) to a human LPA1-stably expressing CHO cell.

FIG. 2H is a graph presenting an evaluation result of a binding property of an antibody (210420-1-H) to a human LPA1-stably expressing CHO cell.

FIG. 2I is a graph presenting an evaluation result of a binding property of an antibody (210420-3-E) to a human LPA1-stably expressing CHO cell.

FIG. 2J is a graph presenting an evaluation result of a binding property of an antibody (211222-1-G) to a human LPA1-stably expressing CHO cell.

FIG. 2K is a graph presenting an evaluation result of a binding property of an antibody (211222-1-A) to a human LPA1-stably expressing CHO cell.

FIG. 3A is a graph presenting an evaluation result of a binding property of an antibody (210309-1-C) to a mouse LPA1-stably expressing CHO cell.

FIG. 3B is a graph presenting an evaluation result of a binding property of an antibody (210309-1-G) to a mouse LPA1-stably expressing CHO cell.

FIG. 3C is a graph presenting an evaluation result of a binding property of an antibody (210309-2-A) to a mouse LPA1-stably expressing CHO cell.

FIG. 3D is a graph presenting an evaluation result of a binding property of an antibody (210309-2-D) to a mouse LPA1-stably expressing CHO cell.

FIG. 3E is a graph presenting an evaluation result of a binding property of an antibody (210309-4-A) to a mouse LPA1-stably expressing CHO cell.

FIG. 3F is a graph presenting an evaluation result of a binding property of an antibody (210310-1-D) to a mouse LPA1-stably expressing CHO cell.

FIG. 3G is a graph presenting an evaluation result of a binding property of an antibody (210420-4-E) to a mouse LPA1-stably expressing CHO cell.

FIG. 3H is a graph presenting an evaluation result of a binding property of an antibody (210420-1-H) to a mouse LPA1-stably expressing CHO cell.

FIG. 3I is a graph presenting an evaluation result of a binding property of an antibody (210420-3-E) to a mouse LPA1-stably expressing CHO cell.

FIG. 3J is a graph presenting an evaluation result of a binding property of an antibody (211222-1-G) to a mouse LPA1-stably expressing CHO cell.

FIG. 3K is a graph presenting an evaluation result of a binding property of an antibody (211222-1-A) to a mouse LPA1-stably expressing CHO cell.

FIG. 4 is a histogram presenting results of flow cytometry performed in Example 6, showing a binding property between an antibody and an endogenous human LPA1.

FIG. 5A is a graph presenting a dose dependency of an inhibitory activity of an antibody (210309-1-C) on human LPA1.

FIG. 5B is a graph presenting a dose dependency of an inhibitory activity of an antibody (210309-1-G) on human LPA1.

FIG. 5C is a graph presenting a dose dependency of an inhibitory activity of an antibody (210309-2-A) on human LPA1.

FIG. 5D is a graph presenting a dose dependency of an inhibitory activity of an antibody (210309-2-D) on human LPA1.

FIG. 5E is a graph presenting a dose dependency of an inhibitory activity of an antibody (210309-4-A) on human LPA1.

FIG. 5F is a graph presenting a dose dependency of an inhibitory activity of an antibody (210310-1-D) on human LPA1.

FIG. 5G is a graph presenting a dose dependency of an inhibitory activity of an antibody (210420-4-E) on human LPA1.

FIG. 5H is a graph presenting a dose dependency of an inhibitory activity of an antibody (210420-1-H) on human LPA1.

FIG. 5I is a graph presenting a dose dependency of an inhibitory activity of an antibody (210420-3-E) on human LPA1.

FIG. 5J is a graph presenting a dose dependency of an inhibitory activity of an antibody (211222-1-G) on human LPA1.

FIG. 5K is a graph presenting a dose dependency of an inhibitory activity of an antibody (211222-1-A) on human LPA1.

FIG. 6A is a graph presenting a dose dependency of an inhibitory activity of an antibody (210309-1-C) on mouse LPA1.

FIG. 6B is a graph presenting a dose dependency of an inhibitory activity of an antibody (210309-1-G) on mouse LPA1.

FIG. 6C is a graph presenting a dose dependency of an inhibitory activity of an antibody (210309-2-A) on mouse LPA1.

FIG. 6D is a graph presenting a dose dependency of an inhibitory activity of an antibody (210309-2-D) on mouse LPA1.

FIG. 6E is a graph presenting a dose dependency of an inhibitory activity of an antibody (210309-4-A) on mouse LPA1.

FIG. 6F is a graph presenting a dose dependency of an inhibitory activity of an antibody (210310-1-D) on mouse LPA1.

FIG. 6G is a graph presenting a dose dependency of an inhibitory activity of an antibody (210420-4-E) on mouse LPA1.

FIG. 6H is a graph presenting a dose dependency of an inhibitory activity of an antibody (210420-1-H) on mouse LPA1.

FIG. 6I is a graph presenting a dose dependency of an inhibitory activity of an antibody (210420-3-E) on mouse LPA1.

FIG. 6J is a graph presenting a dose dependency of an inhibitory activity of an antibody (211222-1-G) on mouse LPA1.

FIG. 6K is a graph presenting a dose dependency of an inhibitory activity of an antibody (211222-1-A) on mouse LPA1.

FIG. 7A is a graph presenting a dose dependency of an inhibitory activity of BMS-986278 on human LPA1.

FIG. 7B is a graph presenting a dose dependency of an inhibitory activity of BMS-986278 on mouse LPA1.

FIG. 8 is a graph presenting an evaluation result of an effect of 210309-4-A on a change in an intracellular cAMP concentration in the absence of LPA1 ligand using a human LPA1-stably expressing CHO cell.

FIG. 9 is a graph presenting a dose-response curve of lysophosphatidic acid at various 210309-4-A concentrations.

FIG. 10A is an explanatory diagram presenting an alignment of a heavy chain variable region of a humanized anti-LPA1 antibody.

FIG. 10B is an explanatory diagram presenting an alignment of a light chain variable region of the humanized anti-LPA1 antibody.

FIG. 11A is a graph presenting an evaluation result of a binding property of an antibody (h309-4-A-BP1) to a human LPA1-stably expressing CHO cell.

FIG. 11B is a graph presenting an evaluation result of a binding property of an antibody (h309-4-A-SS1) to a human LPA1-stably expressing CHO cell.

FIG. 11C is a graph presenting an evaluation result of a binding property of an antibody (h309-4-A-SG1) to a human LPA1-stably expressing CHO cell.

FIG. 11D is a graph presenting an evaluation result of a binding property of an antibody (309-4-A-Chimera) to a human LPA1-stably expressing CHO cell.

FIG. 11E is a graph presenting an evaluation result of a binding property of an antibody (210309-4-A) to a human LPA1-stably expressing CHO cell.

FIG. 12A is a graph presenting a dose dependency of an inhibitory activity of an antibody (h309-4-A-BP1) on human LPA1.

FIG. 12B is a graph presenting a dose dependency of an inhibitory activity of an antibody (h309-4-A-SS1) on human LPA1.

FIG. 12C is a graph presenting a dose dependency of an inhibitory activity of an antibody (h309-4-A-SG1) on human LPA1.

FIG. 12D is a graph presenting a dose dependency of an inhibitory activity of an antibody (309-4-A-Chimera) on human LPA1.

FIG. 12E is a graph presenting a dose dependency of an inhibitory activity of an antibody (210309-4-A) on human LPA1.

### BEST MODE FOR CARRYING OUT THE INVENTION

In the present disclosure, the complementarity determining region is abbreviated as CDR. In the present disclosure, a heavy chain variable region, a heavy chain constant region, a light chain variable region, and a light chain constant region are abbreviated as VH, CH, VL, and CL respectively in some cases. In the present disclosure, the term “antibody” may be synonymous with “immunoglobulin”. The term “nucleic acid” in the present disclosure may be synonymous with “DNA” or “gene”.

### <Human LPA1>

Lysophosphatidic acid receptor 1 (LPA1) is a type of G protein coupled receptors (GPCR), which penetrates cell membranes seven times and has an N-terminal extracellularly oriented and a C-terminal intracellularly oriented. A gene (cDNA) coding for human LPA1 has already been isolated, and an amino acid sequence of human LPA1 is also known. Sequence information of LPA1 can be obtained from gene databases (e.g., National Center for Biotechnology Information (NCBI) Reference Sequence: NP001392). As an example, the amino acid sequence of human LPA1 is represented by SEQ ID NO: 111.

Each domain in human LPA1 is considered to correspond to each of the following parts in the amino acid sequence represented by SEQ ID NO: 111. The amino acid numbers are described on the left side, and domains are described on the right side. Each boundary between the domains may be slightly variable.
1 to 50: N-terminal domain
76 to 83: Intracellular first loop domain
108 to 121: Extracellular first loop domain
145 to 163: Intracellular second loop domain
185 to 204: Extracellular second loop domain
226 to 255: Intracellular third loop domain
281 to 294: Extracellular third loop domain
316 to 364: C-terminal domain

As human LPA1, various variants such as amino acid substitutes are known in addition to the amino acid sequence represented by SEQ ID NO: 111. The “human LPA1” in the present disclosure includes the aforementioned variants as long as the variants have extracellular domains and LPA1 functions.

A gene (cDNA) coding for human LPA2 has already been isolated, and the amino acid sequence of human LPA2 is also known. Sequence information of LPA2 can be obtained from gene databases (NP_004711, SEQ ID NO: 119). A gene (cDNA) coding for human LPA3 has already been isolated, and the amino acid sequence of human LPA3 is also known. The sequence information of LPA3 can be obtained from gene databases (NP_036284, SEQ ID NO: 121).

### <Anti-LPA1 Antibody>

The antibody disclosed in the present application specifically binds to an extracellular domain of human LPA1 and does not specifically bind to extracellular domains of human LPA2 and human LPA3.

An antibody according to an embodiment includes:
a heavy chain CDR1 including an amino acid sequence represented by SEQ ID NO: 1, 11, 21, 31, 41, 51, 61, 71, 81, 91, or 101;
a heavy chain CDR2 including an amino acid sequence represented by SEQ ID NO: 2, 12, 22, 32, 42, 52, 62, 72, 82, 92, or 102;
a heavy chain CDR3 including an amino acid sequence represented by SEQ ID NO: 3, 13, 23, 33, 43, 53, 63, 73, 83, 93, or 103;
a light chain CDR1 including an amino acid sequence represented by SEQ ID NO: 4, 14, 24, 34, 44, 54, 64, 74, 84, 94, or 104;
a light chain CDR2 including an amino acid sequence represented by SEQ ID NO: 5, 15, 25, 35, 45, 55, 65, 75, 85, 95, or 105; and
a light chain CDR3 including an amino acid sequence represented by SEQ ID NO: 6, 16, 26, 36, 46, 56, 66, 76, 86, 96, or 106.

In an antibody according to an embodiment, the heavy chain CDRs 1 to 3 satisfy any one of the above (A1) to (A11).

In an antibody according to an embodiment, the light chain CDRs 1 to 3 satisfy any one of the above (B1) to (B11).

In an antibody according to an embodiment, the heavy chain CDRs 1 to 3 and the light chain CDRs 1 to 3 satisfy any one of the above (AB1) to (AB11).

In an antibody according to an embodiment, the heavy chain variable region and the light chain variable region satisfy any one of the above (C1) to (C11).

The heavy chain variable region (SEQ ID NO: 7) identified in (C1) includes the heavy chain CDRs 1 to 3 (SEQ ID NO: 1 to 3) identified in (A1) or (AB1). The light chain variable region (SEQ ID NO: 9) identified in (C1) includes the light chain CDRs 1 to 3 (SEQ ID NO: 4 to 6) identified in (B1) or (AB1). Examples of an antibody satisfying (A1), (B1), (AB1), or (C1) include “210309-4-A” described in Examples below.

The heavy chain variable region (SEQ ID NO: 17) identified in (C2) includes the heavy chain CDRs 1 to 3 (SEQ ID NO: 11 to 13) identified in (A2) or (AB2). The light chain variable region (SEQ ID NO: 19) identified in (C2) includes the light chain CDRs 1 to 3 (SEQ ID NO: 14 to 16) identified in (B2) or (AB2). Examples of an antibody satisfying (A2), (B2), (AB2), or (C2) include “210309-1-C” described in Examples below.

The heavy chain variable region (SEQ ID NO: 27) identified in (C3) includes the heavy chain CDRs 1 to 3 (SEQ ID NO: 21 to 23) identified in (A3) or (AB3). The light chain variable region (SEQ ID NO: 29) identified in (C3) includes the light chain CDRs 1 to 3 (SEQ ID NO: 24 to 26) identified in (B3) or (AB3). Examples of an antibody satisfying (A3), (B3), (AB3), or (C3) include “210309-1-G” described in Examples below.

The heavy chain variable region (SEQ ID NO: 37) identified in (C4) includes the heavy chain CDRs 1 to 3 (SEQ ID NO: 31 to 33) identified in (A4) or (AB4). The light chain variable region (SEQ ID NO: 39) identified in (C4) includes the light chain CDRs 1 to 3 (SEQ ID NO: 34 to 36) identified in (B4) or (AB4). Examples of an antibody satisfying (A4), (B4), (AB4), or (C4) include “210309-2-A” described in Examples below.

The heavy chain variable region (SEQ ID NO: 47) identified in (C5) includes the heavy chain CDRs 1 to 3 (SEQ ID NO: 41 to 43) identified in (A5) or (AB5). The light chain variable region (SEQ ID NO: 49) identified in (C5) includes the light chain CDRs 1 to 3 (SEQ ID NO: 44 to 46) identified in (B5) or (AB5). Examples of an antibody satisfying (A5), (B5), (AB5), or (C5) include “210309-2-D” described in Examples below.

The heavy chain variable region (SEQ ID NO: 57) identified in (C6) includes the heavy chain CDRs 1 to 3 (SEQ ID NO: 51 to 53) identified in (A6) or (AB6). The light chain variable region (SEQ ID NO: 59) identified in (C6) includes the light chain CDRs 1 to 3 (SEQ ID NO: 54 to 56) identified in (B6) or (AB6). Examples of an antibody satisfying (A6), (B6), (AB6), or (C6) include “210310-1-D” described in Examples below.

The heavy chain variable region (SEQ ID NO: 67) identified in (C7) includes the heavy chain CDRs 1 to 3 (SEQ ID NO: 61 to 63) identified in (A7) or (AB7). The light chain variable region (SEQ ID NO: 69) identified in (C7) includes the light chain CDRs 1 to 3 (SEQ ID NO: 64 to 66) identified in (B7) or (AB7). Examples of an antibody satisfying (A7), (B7), (AB7), or (C7) include “210420-4-E” described in Examples below.

The heavy chain variable region (SEQ ID NO: 77) identified in (C8) includes the heavy chain CDRs 1 to 3 (SEQ ID NO: 71 to 73) identified in (A8) or (AB8). The light chain variable region (SEQ ID NO: 79) identified in (C8) includes the light chain CDRs 1 to 3 (SEQ ID NO: 74 to 76) identified in (B8) or (AB8). Examples of an antibody satisfying (A8), (B8), (AB8), or (C8) include “210420-1-H” described in Examples below.

The heavy chain variable region (SEQ ID NO: 87) identified in (C9) includes the heavy chain CDRs 1 to 3 (SEQ ID NO: 81 to 83) identified in (A9) or (AB9). The light chain variable region (SEQ ID NO: 89) identified in (C9) includes the light chain CDRs 1 to 3 (SEQ ID NO: 84 to 86) identified in (B9) or (AB9). Examples of an antibody satisfying (A9), (B9), (AB9), or (C9) include “210420-3-E” described in Examples below.

The heavy chain variable region (SEQ ID NO: 97) identified in (C10) includes the heavy chain CDRs 1 to 3 (SEQ ID NO: 91 to 93) identified in (A10) or (AB10). The light chain variable region (SEQ ID NO: 99) identified in (C10) includes the light chain CDRs 1 to 3 (SEQ ID NO: 94 to 96) identified in (B10) or (AB10). Examples of an antibody satisfying (A10), (B10), (AB10), or (C10) include “211222-1-G” described in Examples below.

The heavy chain variable region (SEQ ID NO: 107) identified in (C11) includes the heavy chain CDRs 1 to 3 (SEQ ID NO: 101 to 103) identified in (A11) or (AB11). The light chain variable region (SEQ ID NO: 109) identified in (C11) includes the light chain CDRs 1 to 3 (SEQ ID NO: 104 to 106) identified in (B11) or (AB11). Examples of an antibody satisfying (A11), (B11), (AB11), or (C11) include “211222-1-A” described in Examples below.

SEQ ID NO: 1, SEQ ID NO: 21, SEQ ID NO: 31, and SEQ ID NO: 41 have a same amino acid sequence. SEQ ID NO: 2 and SEQ ID NO: 32 have a same amino acid sequence. SEQ ID NO: 6, SEQ ID NO: 16, SEQ ID NO: 26, SEQ ID NO: 36, and SEQ ID NO: 46 have a same amino acid sequence. SEQ ID NO: 15, SEQ ID NO: 25, SEQ ID NO: 35, SEQ ID NO: 45, SEQ ID NO: 65, SEQ ID NO: 75, SEQ ID NO: 85, and SEQ ID NO: 105 have a same amino acid sequence. SEQ ID NO: 24, SEQ ID NO: 34, SEQ ID NO: 54, SEQ ID NO: 94, and SEQ ID NO: 104 have a same amino acid sequence. SEQ ID NO: 51, SEQ ID NO: 91, and SEQ ID NO: 101 have a same amino acid sequence. SEQ ID NO: 52 and SEQ ID NO: 92 have a same amino acid sequence. SEQ ID NO: 53, SEQ ID NO: 93, and SEQ ID NO: 103 have a same amino acid sequence. SEQ ID NO: 55 and SEQ ID NO: 95 have a same amino acid sequence. SEQ ID NO: 56, SEQ ID NO: 66, SEQ ID NO: 76, SEQ ID NO: 86, SEQ ID NO: 96, and SEQ ID NO: 106 have a same amino acid sequence. SEQ ID NO: 61, SEQ ID NO: 71, and SEQ ID NO: 81 have a same amino acid sequence. SEQ ID NO: 62, SEQ ID NO: 72, and SEQ ID NO: 82 have a same amino acid sequence. SEQ ID NO: 63, SEQ ID NO: 73, and SEQ ID NO: 83 have a same amino acid sequence. SEQ ID NO: 64, SEQ ID NO: 74, and SEQ ID NO: 84 have a same amino acid sequence.

The above antibody may be a functional fragment of an antibody. Herein, the “functional fragment of an antibody” refers to a partial fragment of an antibody (i.e. immunoglobulin), which retains at least one action against antigens. Examples of the partial fragment include F(ab′)2, Fab, Fv, disulfide-bonded Fv, single-stranded antibodies (scFv, VH-VL), VH, and polymers thereof, as well as a fusion thereof with the heavy chain CH3 region, and also include various CDRs such as CDR1, CDR2, and CDR3, and a linked assembly of these CDRs, as well as a fusion of these CDRs or the CDR linked assembly with a heavy chain CH3 region. That means, the antibodies according to the present disclosure include partial fragments of antibodies as described above. A partial fragment of the antibody is referred to as an “antibody fragment” in some cases.

When the aforementioned antibody is a functional fragment, the antibody has, for example, the following effects. That means, when the anti-LPA1 antibody according to the present disclosure is applied for a medicament as described below, use of a full-length antibody such as IgG type may cause damage to a target tissue leading to side effects, in addition to inhibited cell functions via a target receptor. In such a case, the side effects can be easily avoided by adopting the “functional fragment of antibody” using only a variable region.

The above antibody may be a multispecific antibody. A multispecific antibody according to this embodiment has at least a first specific binding property that the antibody binds specifically to the extracellular domain of human LPA1 and not specifically to the extracellular domains of human LPA2 and human LPA3, and a second specific binding property different from the first specific binding property. The second specific binding property may be targeted to human LPA1 or to other target molecules. Examples of the multispecific antibody include a diabody as a type of bispecific antibodies.

The class (isotype) of the above antibodies is not particularly limited. For example, the antibodies may belong to any class of IgG, IgM, IgA, IgD, IgE and the like. Furthermore, the subclass of the above antibodies is not particularly limited, neither. For example, if the antibody belongs to the IgG class, the subclass thereof may be any of IgG1, IgG2, IgG3, IgG4, and the like.

In a preferable embodiment, the above antibodies have an activity of blocking LPA1-dependent cell functions. The LPA1-dependent cell functions are induced through activation of a protein such as trimeric G proteins and β-arrestin, which is conjugated to an intracellular portion of LPA1 to act for intracellular signaling. The LPA1-dependent activation of the cell functions is induced by stimulation of an LPA1 ligand or an LPA1 ligand-independent high expression of LPA1. Examples of the cell functions include a change in intracellular cyclic adenosine monophosphate (cAMP), a change in intracellular calcium ions, binding of guanosine triphosphate (GTP) to a low molecular weight G protein Rho, cell proliferation, cell migration, production of cytokines and chemokines, and cell transformation.

The extracellular domain of LPA1 to which the above antibodies specifically bind may be any of an N-terminal domain, an extracellular first loop domain, an extracellular second loop domain, and an extracellular third loop domain. The above antibodies may bind to only one of these extracellular domains or to two or more of them.

The present disclosure encompasses antibodies that are “functionally equivalent” to the anti-LPA1 antibodies described above. The functionally equivalent antibodies include antibodies having the same epitopes as of the anti-LPA1 antibodies. For example, epitopes of eleven types of anti-LPA1 antibodies specifically described in Examples below are analyzed by an epitope mapping method using a partial peptide of LPA1 or the like. Then, a synthetic peptide including identified epitopes can be used as an antigen to obtain an anti-LPA1 antibody that binds to the same epitopes as the above eleven anti-LPA1 antibodies. Furthermore, amino acid sequences of a heavy chain variable region and a light chain variable region of the obtained anti-LPA1 antibody can be determined to identify amino acid sequences of the heavy chain CDRs 1 to 3 and the light chain CDRs 1 to 3.

As an example of the antibodies functionally equivalent to the above antibodies, the present disclosure encompasses an antibody that binds specifically to the extracellular domain of human LPA1 and not specifically to the extracellular domains of human LPA2 and human LPA3 and satisfies any one of the (C1′) to (C11′) described above. In (C1′) to (C11′), the number of substituted, added, or deleted amino acids is preferably 1 to 8, more preferably 1 to 5, particularly preferably 1 to 3. The identity of the amino acid sequences described above is preferably 92% or higher, more preferably 95% or higher, and particularly preferably 97% or higher. In a preferable embodiment, the antibody has an activity of blocking the LPA1-dependent cell functions.

Examples of a method for determining whether epitopes are identical between two antibodies include a method using a competition experiment. For example, if binding between the above eleven anti-LPA1 antibodies or functional fragments thereof as first antibodies and the receptors is competitively inhibited by second antibodies to be tested, the second antibodies are considered to bind to the same epitopes as for the first antibodies. As an example of the antibodies that are functionally equivalent to the above antibodies, the present disclosure encompasses second antibodies that bind specifically to the extracellular domain of human LPA1 and not specifically to the extracellular domains of human LPA2 and human LPA3, and competitively inhibit the binding between the above antibodies (first antibodies) and the receptors. In a preferable embodiment, the second antibodies have an activity of blocking the LPA1-dependent cell functions.

### <Humanized Antibody>

The present disclosure encompasses the above antibodies that are humanized antibodies. The humanized antibodies refer to antibodies in which the CDRs are derived from non-human animals and other regions (e.g., framework regions and constant regions) are derived from humans. Methods for constructing and producing humanized antibodies are described later.

### <Chimeric Antibody>

The present disclosure encompasses the above antibodies that are chimeric antibodies. The chimeric antibodies refer to antibodies in which the heavy chain variable region (VH) and light chain variable region (VL) are derived from non-human animals, and other regions such as the heavy chain constant region (CH) and light chain constant region (CL) are derived from humans. Methods for constructing and producing chimeric antibodies are described later.

### <Nucleic Acid>

The present disclosure encompasses nucleic acids (DNA) encoding the above antibodies. The nucleic acids include, e.g., a first nucleic acid encoding the heavy chain variable region and/or a second nucleic acid encoding the light chain variable region.

The heavy chain variable region which the first nucleic acid encodes includes, e.g., a heavy chain CDR1 including an amino acid sequence represented by SEQ ID NO: 1, 11, 21, 31, 41, 51, 61, 71, 81, 91, or 101, a heavy chain CDR2 including an amino acid sequence represented by SEQ ID NO: 2, 12, 22, 32, 42, 52, 62, 72, 82, 92, or 102, and a heavy chain CDR3 including an amino acid sequence represented by SEQ ID NO: 3, 13, 23, 33, 43, 53, 63, 73, 83, 93, or 103. The heavy chain variable region which the first nucleic acid encodes includes, e.g., the heavy chain CDRs 1 to 3 identified in any one of (A1) to (A11) above. The heavy chain variable region which the first nucleic acid encodes is identified e.g. in any one of (C1) to (C11) above. The heavy chain variable region which the first nucleic acid encodes is identified e.g. in any one of (C1′) to (C11′) above.

The light chain variable region which the second nucleic acid encodes includes, e.g., a light chain CDR1 including an amino acid sequence represented by SEQ ID NO: 4, 14, 24, 34, 44, 54, 64, 74, 84, 94, or 104, a light chain CDR2 including an amino acid sequence represented by SEQ ID NO: 5, 15, 25, 35, 45, 55, 65, 75, 85, 95, or 105, and a light chain CDR3 including an amino acid sequence represented by SEQ ID NO: 6, 16, 26, 36, 46, 56, 66, 76, 86, 96, or 106. The light chain variable region which the second nucleic acid encodes includes, e.g., heavy chain CDRs 1 to 3 identified in any one of (B1) to (B11) above. The light chain variable region which the second nucleic acid encodes is identified e.g. in any one of (C1) to (C11) above. The light chain variable region which the second nucleic acid encodes is identified in e.g., any one of (C1′) to (C11′) above.

The above nucleic acids may be incorporated into a vector. The vector is selected as appropriate depending on a type or the like of a host cell to be transduced with the vector. Examples of the vector include a gene therapy vector. In the case of a gene therapy vector, the vector itself can be directly administered into a living body.

### <Cell>

The present disclosure encompasses a cell containing the above nucleic acids. Examples of the cell include a cell containing a vector into which the above nucleic acids are incorporated. The type of the cell is not particularly limited as long as the cell can express the above nucleic acids, e.g., the above vector is functional in the cell. Examples of the cell include animal cells (e.g., COS cells, CHO cells), yeast, bacteria (e.g., E. coli), plant cells, and insect cells.

### <Method for Producing Antibody>

The above antibodies can be produced using a genetic recombination method. That means, recombinant cells that express the above nucleic acids can be constructed to obtain the above antibodies from culture medium of the cells.

### <Construction and Preparation of Humanized Antibody>

A method for constructing and producing humanized antibodies will be explained. As an example, a method for constructing and producing humanized antibodies having the heavy chain CDRs 1 to 3 and the light chain CDRs 1 to 3 identified in (AB1) above will be described. As DNAs encoding each CDR, DNAs coding for the amino acid sequences presented in SEQ ID NO: 1 to 6 are first prepared. The DNAs can be prepared by using a known method such as PCR. The DNA can also be prepared by chemical synthesis.

Next, these DNAs are used to prepare DNAs encoding variable regions in which the heavy chain CDRs 1 to 3 are implanted into a framework region (FR) of a VH in any human antibody. Similarly, DNAs coding for variable regions in which the light chain CDRs 1 to 3 are implanted into an FR of a VL in any human antibody are prepared. Each DNA prepared is inserted into a vector having sequences coding for a CH or CL of a human antibody to construct a humanized antibody expression vector. The constructed expression vector is introduced into a host cell to obtain a recombinant cell that expresses a humanized antibody. Then, this recombinant cell is cultured to obtain a desired humanized antibody from the culture supernatant.

Humanized antibodies having the heavy chain CDRs 1 to 3 and light chain CDRs 1 to 3 identified in (AB2) to (AB11) above can be constructed and produced by the same method.

### <Construction and Preparation of Chimeric Antibody>

A method for constructing and producing a chimeric antibody will be explained. As an example, a method for constructing and producing a chimeric antibody having the heavy chain variable region (VH) and light chain variable region (VL) identified in (C1) above will be described. As a DNA coding for the VL, a DNA encoding the amino acid sequence represented by SEQ ID NO: 7 is first prepared. As a DNA encoding the VL, a DNA encoding the amino acid sequence represented by SEQ ID NO: 9 is prepared. The DNA can be prepared by using a known method such as PCR. The DNA can also be prepared by chemical synthesis.

The obtained DNAs encoding the VH or VL are each inserted into a vector having a sequence coding for the CH or CL respectively of a human antibody to construct a chimeric antibody expression vector. The vector having the sequence encoding the CH or CL of the human antibody can be commercially available. The constructed expression vector is introduced into a host cell to obtain a recombinant cell that expresses a chimeric antibody. Then, the recombinant cell is cultured to obtain a desired chimeric antibody from the culture supernatant.

Chimeric antibodies having the VH and VL identified in (C2) to (C11) above can also be constructed and produced by the same method.

### <Method for Producing Multispecific Antibody>

As a method for producing a multispecific antibody, for example, the anti-human LPA1 antibody according to the present disclosure or a fragment thereof is linked with another antibody or a fragment thereof. As another example, the anti-human LPA1 antibody according to the present disclosure or a fragment thereof and another antibody or a fragment thereof are co-expressed in a host cell. Examples of other linking methods include chemical coupling, gene fusion, and non-covalent association.

Examples of practically used multispecific antibodies (bispecific antibodies) include BLINCYTO® that binds to CD3 and CD19. Other examples include HEMLIBRA® that binds to coagulation factor IXa and coagulation factor X. The techniques for producing multispecific antibodies are known in the art, and these known production techniques can also be applied to the anti-human LPA1 antibody according to the present disclosure.

### <Purification Method>

The method for purifying the above antibodies is not particularly limited, and any known method can be used. For example, a culture supernatant of the above recombinant cell can be collected to purify the above antibodies by combining known methods such as various types of chromatography, salt precipitation, dialysis, and membrane separation. If the isotype of the antibody is IgG, the antibody can also be easily purified by affinity chromatography using protein A.

### <Modified Antibody>

The antibodies according to the present disclosure may be modified antibodies bonded with other molecules. Examples of other molecules include peptides, proteins, low molecular weight compounds, radioisotopes, and light absorbers. Examples of the method for bonding the antibodies with other molecules include chemical coupling, gene fusion, and non-covalent association. If other molecules to be bonded are antibodies, the modified antibodies can be multispecific antibodies. In this regard, multispecific antibodies can be regarded as a type of modified antibodies.

In a preferable embodiment, the above modified antibodies are antibody drug conjugates (ADC). Examples of the drug to be conjugated include anti-fibrosis agents; low molecular weight anticancer drugs (cytotoxins, chemotherapeutic drugs); biologically active proteins or polypeptides such as cytokines; radioisotopes; and light absorbers. These drugs can be conjugated with the antibodies directly or indirectly via linkers or chelating agents to produce antibody drug conjugates.

Examples of practically used antibody drug conjugates include: ENHERTZ® containing trastuzumab bonded with a camptothecin derivative, as an antibody drug conjugate prepared by conjugation of the antibody with a low molecular weight anticancer drug; ZEVALIN® containing an anti-CD20 monoclonal antibody labeled with yttrium 90 (⁹⁰Y), as an antibody drug conjugate prepared by conjugation of the antibody with a radioisotope; and ACALUX® containing cetuximab bonded with IR Dye 700DX, as an antibody drug conjugate prepared by conjugation of the antibody with a light absorber. The drugs contained in these antibody drug conjugates can also be applied to the anti-human LPA1 antibody according to the present disclosure. Furthermore, examples of suitable drugs used for the antibody drug conjugates are known in the art, and these known drugs can also be applied to the anti-human LPA1 antibody according to the present disclosure.

### <Medicament>

The present disclosure encompasses a medicament containing the above antibody as an active ingredient. The medicament may be a pharmaceutical composition containing the above antibody and a pharmaceutically acceptable carrier. Preferably, the medicament blocks the LPA1-dependent cell functions.

In a preferable embodiment, the above medicament is used for treating a disease, a disorder, or a symptom, causally involving the LPA1-dependent cell dysfunction. Examples of the disease, the disorder, or the symptom, causally involving the LPA1-dependent cell dysfunction include fibroses selected from a group consisting of hepatic fibrosis, kidney fibrosis, pulmonary fibrosis, skin fibrosis, cardiovascular fibrosis, gastrointestinal fibrosis, and other fibrous diseases.

Examples of the liver fibrosis include fibrosis selected from a group consisting of nonalcoholic steatohepatitis (NASH), nonalcoholic fatty liver disease (NAFLD), liver cirrhosis, ischemia reperfusion, post liver transplantation disorder, necrotizing hepatitis, hepatitis B, hepatitis C, primary biliary cirrhosis, and primary sclerosing cholangitis. The liver cirrhosis is caused, e.g., by at least one selected from a group consisting of alcoholic induction, drug induction, and chemical induction.

Examples of the kidney fibrosis include fibroses selected from a group consisting of proliferative glomerulonephritis, sclerosing glomerulonephritis, nephrogenic fibrosing dermopathy, diabetic nephropathy, renal tubulointerstitial fibrosis, and focal segmental glomerulosclerosis.

Examples of the pulmonary fibrosis include fibroses selected from a group consisting of lung interstitial fibrosis, drug-induced sarcoidosis, pulmonary fibrosis, idiopathic pulmonary fibrosis, asthma, chronic obstructive lung disease, diffuse alveolar damage disease, pulmonary hypertension, and neonatal bronchopulmonary malformation.

Examples of the skin fibrosis include fibroses selected from a group consisting of scleroderma, keloid scarring, psoriasis, hypertrophic scarring, and pseudo-scleroderma.

Examples of the cardiovascular fibrosis include fibroses selected from a group consisting of atherosclerosis, coronary artery restenosis, congestive cardiomyopathy, heart failure, heart transplantation, and myocardial fibrosis.

Examples of the gastrointestinal fibrosis include fibroses selected from a group consisting of collagenous colitis, villous atrophy, crypt hyperplasia, polyp formation, Crohn's fibrosis, gastric ulcer healing, or scar after abdominal adhesion surgery.

The fibrosis may be caused by a bone-related fiberization disease and have rheumatoid pannus formation.

Other examples of the disease, the disorder, and the symptom, causally involving the LPA1-dependent cell dysfunction include cell proliferative diseases such as hematological cancer and solid carcinoma (tumor cell proliferation, tumor infiltration and metastasis, and controlled angiogenesis).

Examples of the hematological cancer include hematological cancers selected from a group consisting of acute myeloid leukemia, acute lymphoblastic leukemia, chronic myeloid leukemia, chronic lymphocytic leukemia, and non-Hodgkin's lymphoma.

Examples of the solid carcinoma include solid carcinomas selected from a group consisting of breast cancer, malignant breast tumor, gastric cancer, melanoma, non-small-cell lung cancer, pulmonary adenocarcinoma, gastric cancer, pancreatic cancer, ovarian cancer, uterine cancer, cervical cancer, hepatoma, prostate cancer, urothelial cancer, renal cell cancer, and various squamous cell cancers. Examples of the squamous cell cancer include squamous cell cancers selected from a group consisting of oral squamous cell carcinoma, esophageal squamous cell carcinoma, and pharyngeal squamous cell carcinoma.

Other examples of the disease, the disorder, or the symptom, causally involving the LPA1-dependent cell dysfunction include: pains including fibromyalgia, cancer pain, and diabetic neuropathy; inflammatory diseases and autoimmune disease, including rheumatoid arthritis, sepsis, chronic obstructive lung disease, inflammatory bowel disease, implanted organ rejection, Guillain-Barre syndrome, and multiple sclerosis; metabolic diseases including obesity and insulin-resistant diabetes; cardiovascular disorders including stroke, hypertensive nephropathy, and Raynaud's phenomenon; nervous system disorders including hydrocephalus, schizophrenia, depression, and dementia; urologic diseases including prostatic hyperplasia and urinary incontinence; and ophthalmological diseases including ischemic retinopathy, diabetic retinopathy, and age-related macular degeneration.

### <Administration Method>

The above medicament can be administered orally or parenterally, and systemically or locally. Examples of the dosage forms include an injection dosage form, a transnasal dosage form, a transpulmonary dosage form, and a transdermal dosage form. In the case of the injection dosage form, the medicament can be administered systemically or locally, e.g., by intravenous injection, intramuscular injection, intraperitoneal injection, subcutaneous injection, or the like. The administration method can be selected as appropriate depending on age and symptoms of a patient. A dose of the above antibodies can be selected within a range of 0.0001 mg to 1000 mg per kilogram of body weight. Alternatively, for example, the dose can be selected within a range of 0.001 to 100,000 mg/body weight. However, the dose of the antibodies is not limited to these ranges.

### <Formulation>

The above medicament can be formulated according to an ordinary method (e.g., Remington's Pharmaceutical Science, latest edition, Mark Publishing Company, Easton, U.S.A.). The medicament can contain pharmaceutically acceptable carriers or additives. Examples of the carriers or the additives include, but are not limited to, surfactants (e.g., polyethylene glycol (PEG), Tween), excipients, antioxidants (e.g., ascorbic acid), colorants, fragrant agents, preservatives, stabilizers, buffers (e.g., phosphoric acid, citric acid, other organic acids), chelating agents (e.g., ethylene diamine tetra acetic acid (EDTA)), suspensions, isotonizing agents, binders, disintegrators, lubricants, fluidity accelerators, and flavoring agents, and other commonly used carriers or the like can be used as appropriate. Specific examples of the carriers or the additives include light anhydrous silicic acid, lactose, crystalline cellulose, mannitol, starch, carmellose calcium, carmellose sodium, hydroxypropyl cellulose, hydroxypropylmethyl cellulose, polyvinylacetal diethylaminoacetate, polyvinylpyrrolidone, gelatin, medium-chain fatty acid triglyceride, polyoxyethylene hardened castor oil 60, saccharose, carboxymethyl cellulose, corn starch, and inorganic salts. The medicament may contain other low molecular weight polypeptides; proteins such as serum albumin, gelatin, and immunoglobulin; and amino acids such as glycine, glutamine, asparagine, arginine, and lysine.

When the medicament is in an aqueous solution for injection, the medicament may be saline, an isotonic solution containing glucose or other auxiliary agents, such as D-sorbitol, D-mannose, D-mannitol, sodium chloride, and they may be used in combination with an appropriate solubilizer, e.g., an alcohol (e.g., ethanol), a polyalcohol (e.g., propylene glycol, PEG), a nonionic surfactant (polysorbate 80, HCO-50), or the like. If necessary, the above antibodies can be encapsulated in microcapsules (e.g., made of hydroxymethyl cellulose, gelatin, poly[methyl methacrylate]) or can be configured as a colloidal drug delivery system (e.g., liposomes, albumin microspheres, microemulsions, nanoparticles, and nanocapsules (e.g., see “Remington's Pharmaceutical Science 16th edition”, Oslo Ed., 1980).

Furthermore, techniques for sustained release of drugs are known and can be applied to the above medicament (Langer et. al., J. Biomed. Master. Res. 15: 162-277 (1981); Langer, Chem. Tech. 12: 9 (8-105) (1982); U.S. Pat. No. 3,773,919; EP Patent No. 58,481; Sidman et. al., Biopolymers 22: 547-556 (1983); E P Patent No. 133,988).

### <Application for Gene Therapy>

The above nucleic acid may be incorporated into a gene therapy vector or an mRNA that can be translated into a protein in vivo, to prepare a gene therapeutic drug. Examples of the method for administering the gene therapeutic drug (recombinant vector) include a direct administration method using naked plasmids, as well as a method in which the drug is packed into liposomes or the like for administration, a method in which the drug is incorporated into various virus vectors such as retrovirus vectors, adenovirus vectors, vaccinia virus vectors, poxvirus vectors, adeno-associated virus vectors, and hemagglutinating virus of Japan (HVJ) vectors (see Adolph, “Viral Genome Methods”, CRC Press, Florid (1996)), and a method in which the drug is applied on bead carriers such as colloidal gold particles for administration (e.g., WO 93/17706).

The gene therapeutic drug may be administered by any method as long as the antibodies are expressed in vivo and can exert their effects. Preferably, a sufficient amount of the drug is administered through an appropriate parenteral route, such as intravenous route, intraperitoneal route, subcutaneous route, intradermal route, intra-adipose route, intramammary route, inhalation route, intramuscular route, or the like by a method using injection, infusion, gas inductive particle bombardment (using e.g., an electron gun), mucosal route using collunarium or the like. Furthermore, the gene therapeutic drug may be administered ex vivo by a process in which the drug is introduced into cells using liposome transfection, particle bombardment (U.S. Pat. No. 4,945,050), or viral infection, and the cells are reintroduced into the animal.

Furthermore, the present disclosure also includes a treatment method and a therapeutic drug regarding a disease of a mammal suffering from a disease or an illness caused by an LPA1-dependent abnormal cell hyperfunction.

Herein, “treatment” means prevention or alleviation of progression and exacerbation of a disease symptom in a mammal that is at risk of suffering from or has suffered from the disease. Thereby, the “treatment” is used as a mean of a therapeutic treatment aimed to prevent or alleviate progression and exacerbation of various symptoms and the like of the disease.

The term “disease” refers to all diseases that develop due to the LPA1-dependent abnormal cell hyperfunction, and conceptually includes, but is not limited to, hepatic fibrosis, kidney fibrosis, pulmonary fibrosis, skin fibrosis, cardiovascular fibrosis, gastrointestinal fibrosis, and other fibrosis diseases, as well as hematological cancer, solid carcinoma, pain, inflammatory diseases, autoimmune diseases, metabolic diseases, cardiovascular disorders, nervous system disorders, urologic diseases, and ophthalmological diseases. The “mammal” to be treated means any animal classified into mammals, and examples thereof include, but are not limited to, humans, as well as pet animals such as dogs, cats, and rabbits, and domestic animals such as cattle, pigs, sheep, and horses. Humans are particularly preferable “mammals”.

### <Antibody-Immobilized Carrier>

The present disclosure encompasses a carrier to which the above antibodies are immobilized (antibody-immobilized carrier). In a preferable embodiment, the antibody-immobilized carrier is used to remove LPA1-expressing cells from a body fluid by bringing the cattier into contact with the body fluid containing the LPA1-expressing cells. Examples of the body fluid include blood. Only one type or two or more types of the antibodies may be immobilized to the carrier.

As a specific form of the antibody-immobilized carrier according to the present disclosure, the antibodies are immobilized to a water-insoluble carrier, which is charged into a container. Any material can be used as the water-insoluble carrier, but examples of the material suitable in terms of formability, sterility, and low cytotoxicity include: synthetic polymers such as polyethylene, polypropylene, polystyrene, acrylic resin, nylon, polyester, polycarbonate, polyacrylamide, and polyurethane; natural polymers such as agarose, cellulose, cellulose acetate, chitin, chitosan, and alginate; inorganic materials such as hydroxyapatite, glass, alumina, and titania; and metal materials such as stainless steel and titanium.

Examples of the shape of the carrier include granular shape, cotton shape, woven fabric shape, non-woven fabric shape, sponge-like porous shape, and flat plate shape, but granular shape, cotton shape, woven fabric shape, non-woven fabric shape, sponge-like porous shape are preferable in that these materials have a large surface area per volume. For example, peripheral blood can be passed through a porous filter prepared by previously charging an antibody-immobilized water-insoluble carrier into a container, to efficiently remove disease-related LPA1-expressing cells.

An LPA1-expressing cell removal kit can be prepared by combining the antibody-immobilized carrier according to the present disclosure with other components. Examples of the other components include an anticoagulant, and an extracorporeal circulation circuit.

### <Other Disclosures>

The present disclosure encompasses a method for treating a disease, disorder, or symptom, including administering an effective amount of the above antibodies to a patient suffering from the disease, disorder, or symptom involving an LPA1-dependent cell dysfunction. The present disclosure encompasses the above antibodies for use in treating a disease, disorder, or symptom involving an LPA1-dependent cell dysfunction. The present disclosure encompasses use of the above antibodies for the manufacture of a medicament for the treatment of a disease, disorder, or symptom involving an LPA1-dependent cell dysfunction. The aforementioned diseases, disorders, or symptoms include at least tissue fibrosis, cell proliferative disease, pain, inflammatory disease, autoimmune disease, metabolic disease, cardiovascular disorder, urologic disease, or ophthalmological disease.

### EXAMPLES

### [Example 1] Mouse Immunization for Preparation of Anti-LPA1 Antibody

### (1) Construction of LPA1-GroEL Subunit Fusion Protein-Expressing Gene Immunization Vector

An artificial gene added with a GCTAGC sequence at the 5′ end and a GTCGAC sequence at the 3′ end was synthesized using, as a base, a human LPA1 (NP_001392, SEQ ID NO: 111) that had been registered in National Center for Biotechnology Information (NCBI) GenBank. This artificial gene was introduced into a cloning vector, and then the resulting vector was cleaved at NheI and Sal sites to prepare DNA fragments. pCI-hCCR7 GroEL (described in WO 2012/043533 and JP Patent No. 5315495) was digested with restriction enzymes NheI and SalI, into which the prepared DNA fragments were inserted, to construct an immunization vector pCI-hLPA1 GroEL. This vector expresses a fusion protein of the human LPA1 and GroEL.

Instead of the human LPA1, a mouse LPA1 (NP_034466, SEQ ID NO: 112) was subjected to the same procedure to construct an immunization vector pCI-mLPA1 GroEL. This vector expresses a fusion protein of the mouse LPA1 and GroEL.

### (2) DNA Immunization

The vector pCI-hLPA1 GroEL or pCI-mLPA1 GroEL was dissolved at a concentration of 2 mg/mL in phosphate-buffered saline (PBS) to prepare an immunization composition. 8-week-old mice of various strains were immunized by dosing 25 μL of this immunization composition to each thigh muscle of all legs using in vivo electroporation (Day 0). Thus, the both legs were dosed with 50 g each of pCI-hLPA1 GroEL or pCI-mLPA1 GroEL, i.e. one mouse was immunized with 100 g of DNA per one intervention. Subsequently, the same DNA immunization was performed on day 14, day 28, day 42, and day 56. Furthermore, blood samples were collected before the immunization and 21, 35, and 63 days after the immunization, to prepare serum.

### (3) Preparation of LPA1-Stably Expressing Cell

An artificial gene was synthesized using, as a base, a human LPA1 (NP_001392, SEQ ID NO: 111) that had been registered in NCBI gene bank, and introduced into a pCIneo vector (Promega), to construct pCIneo-hLPA1. pCIneo-hLPA1 was introduced into CHO-K1 cell using Lipofectamine 2000 (Thermo Fisher Scientific, Inc.). The resulting cells were cultured in a culture medium containing G418 (Promega) to clone G418-resistant cells that stably expressed human LPA1 (hereinafter referred to as “human LPA1-stably expressing CHO cells”).

An artificial gene was synthesized using, as a base, a mouse LPA1 (NP_034466, SEQ ID NO: 112) that had been registered in NCBI gene bank, and introduced into pEF5/FRT/V5-DEST (Thermo Fisher Scientific, Inc.) to construct pEF-FRT-mLPA1. pEF-FRT-mLPA1 and pOG44 plasmid (Thermo Fisher Scientific, Inc.) were simultaneously introduced into Flp-In—CHO cell (Thermo Fisher Scientific, Inc.) using Lipofectamine 2000. The resulting cell was cultured in a culture medium containing hygromycin (Thermo Fisher Scientific, Inc.) to clone a hygromycin-resistant cell that stably expressed mouse LPA1 (hereinafter referred to as “mouse LPA1-stably expressing CHO cells”).

### (4) Evaluation for Binding Property of Serum Antibody to Human LPA1 and Mouse LPA1 by Flow Cytometry

The human LPA1-stably expressing CHO cell, mouse LPA1-stably-expressing CHO cell, and a CHO-K1 cell or CHO-FlpIn cell (hereinafter referred to as “negative control cell”) were washed with a fluorescence-activated cell sorting (FACS) buffer (PBS containing 1% of fetal bovine serum (FBS)), to which Fc Block (Cytek Biosciences, Inc.) was added in an amount of 1/500 of the cell suspension to block the cells at 4° C. for 30 minutes. After the blocking, each cell was incubated with 50 time-diluted pre- and post-immunization serums. Furthermore, each cell was washed with FACS buffer, to which a phycoerythrin-labeled anti-mouse IgG antibody (SouthernBiotech) was added as a secondary antibody. Then, the binding of the anti-human LPA1 antibody and anti-mouse LPA1 antibody in serum to each cell was evaluated using a flow cytometer iQue (Sartorius AG). The serum after the immunization contained antibodies that bound to the human LPA1-stably expressing CHO cell or the mouse LPA1-stably expressing CHO cell but did not bind to the negative control cells. Individuals with increased specific antibody titers against LPA1 were boosted and sampled.

### (5) Boosting Using Transient Expression Cell and Sampling

HEK293FT cell (Thermo Fisher Scientific, Inc.) was transfected with the vector pCI-hLPA1 or pCI-mLPA1 using Lipofectamine 2000 to transiently express human LPA1 or mouse LPA1. This cell was injected into spleen and abdominal cavities of individuals with an increased antibody titer, and 3 days later, spleen, inguinal lymph node, and iliac lymph node were sampled. Splenocytes and lymph node-derived cells were separated from each tissue sample, then suspended in CELLBANKER 1plus (Nippon Zenyaku Kogyo Co., Ltd.), and stored at −80° C. until sorting of specific antibody-producing lymphocytes.

### [Example 2] Preparation of Antibody

### (1) Selection of Specific Antibody-Producing Lymphocyte Using Microwell

The specific antibody-producing lymphocytes were selected using microwells in accordance with a method described in WO 2020/171020 and JP Patent No. 6881801. That means, the human LPA1-stably expressing CHO cell or the mouse LPA1-stably expressing CHO cell was respectively suspended in F-12 culture medium (10% FBS, containing Penicillin/Streptomycin) to prepare a cell suspension at 3.5×10⁵ cells/500 μL. The cell suspension was put into a microchamber (AS ONE Corporation.) for cell picking systems. The microchamber was subjected to centrifugation at 300 rpm for 1 minute five times to prepare the suspension such that one or two human LPA1-stably expressing CHO cells or mouse LPA1-stably expressing CHO cells were contained in each microwell. The micro chamber was washed with F-12 culture medium, to which 500 μL of F-12 culture medium was added, which was incubated in a CO₂ incubator at 37° C. for 1 h so that the human LPA1-stably expressing CHO cell or the mouse LPA1-stably expressing CHO cell adhered to bottom faces of the microwells. To the microwells, CytoRed solution (Dojin Molecular Technologies, Inc.) in a concentration adjusted to 10 nM by F-12 culture medium was added, which was incubated at 37° C. for another 1 hour to stain the CHO cells. The microchamber was washed with F-12 culture medium three times to remove excess CytoRed, and then 1 mL of F-12 medium was charged into the microchamber.

Antibody-producing cells were concentrated from the cells collected in Example 1 (5) after the DNA immunization using EasySep Mouse Biotin Positive Selection Kit (STEMCELL Technologies). In 500 μL of F-12 culture medium, 1.1×105 antibody-producing cells were suspended and charged into a microchamber. The microchamber was subjected to centrifugation at 300 rpm for 1 minute five times to prepare the suspension such that one or two antibody-producing cells were contained in each microwell. The micro chamber was washed with the culture medium, to which an appropriate amount of culture medium was added, which was incubated at 37° C. for 30 minutes to secrete antibodies from the antibody producing cells. The microwells were washed to remove the supernatant, to which Alexa Fluor 488-labeled anti-mouse IgG antibody (Abcam) diluted by 500 times in RPMI 1640 (containing 10% FBS) was added and incubated at 37° C. for 30 minutes. The microwells were washed with RPMI 1640 (phenol red-free, containing 1% FBS) three times, to which 1 mL of RPMI1640 was added. The microchamber was set to a cell picking system (AS ONE Corporation.) to acquire information on a transmitted light image and two fluorescence images of all microwells. Fluorescence of CytoRed was detected at an excitation wavelength of 543 nm and a fluorescent wavelength of 593 nm. Fluorescence of Alexa Fluor 488 was detected at an excitation wavelength of 482 nm and a fluorescent wavelength of 536 nm. From microwells that could be determined to have antibodies binding to the surfaces of the human LPA1-stably expressing CHO cells or mouse LPA1-stably expressing CHO cells based on scan images of transmitted light, CytoRed, and Alexa Fluor 488, antibody-producing cells were collected in the cell lysate using a capillary with a diameter of several m to several tens of m (AS ONE Corporation.)

(2) Isolation of Antibody Gene from Collected Antibody-Producing Cell

An antibody gene was obtained from the antibody producing cells by MAGrahd method (Kurosawa N, Yoshioka M, Fujimoto R, Yamagishi F, Isobe M. “Rapid production of antigen-specific monoclonal antibodies from a variety of animals”, BMC Biol. 2012; 10: 80). In other words, a cell-derived mRNA was captured on an oligo dT magnet by mixing 5 μL of cell lysate collected in (1) with 5 g of oligo dT magnet. The oligo dT magnet was washed with a washing solution using MAGrahd reactor tray and neodymium magnet, and then a cDNA was synthesized by a reverse transcription reaction. The magnet was further washed, and then a 5′-terminal translational reaction was performed. Using the synthesized cDNA, genes on the antibody heavy chain variable region (VH region) and genes on the antibody light chain variable region (VL region) were isolated and amplified by 5′ racePCR. In order to enhance the specificity of the amplified product, PCR was repeated twice. In the first PCR, a first forward primer (SEQ ID NO: 113) that amplified the VH and VL regions in common, a VH first reverse primer (SEQ ID NO: 114) that specifically amplified the VH region, and a VL first reverse primer (SEQ ID NO: 115) that specifically amplified the VL region were mixed and used. In the second PCR, the amplified product in the first PCR was used as a template, and, for amplification of the VH region, a second forward primer (SEQ ID NO: 116) and a VH second reverse primer (SEQ ID NO: 117) that specifically amplified the VH region were used as primers, and for amplification of the VL region, a second forward primer (SEQ ID NO: 116) and a VL second reverse primer (SEQ ID NO: 118) that specifically amplified the VL region were used as primers. As a result of an agarose gel electrophoresis for the sample after the second PCR, a gene amplified product corresponding to the VH region was found at a position of approximately 750 bp, and a gene amplified product corresponding to the VL region was found at a position of approximately 550 bp.

### (3) Construction of Antibody Expression Unit

An antibody expression unit was constructed by TS-jPCR method (Yoshioka M, Kurosawa N, Isobe M. “Target-selective joint polymerase chain reaction: a robust and rapid method for high-throughput production of recombinant monoclonal antibodies from single cells.”, BMC Biotechnol., 2011; 11: 75). That means, the VH region gene amplified in (2), an antibody heavy chain constant region gene, and a gene including a promoter region necessary for gene expression were fused by PCR to construct an antibody expression unit that expressed a full-length antibody heavy chain. Similarly, the VL region gene amplified in (2), an antibody light chain constant region gene, and a gene including a promoter region necessary for gene expression were fused by PCR to construct an antibody expression unit that expressed a full-length antibody light chain.

(4) Introduction of Antibody Expression Unit into Mammalian Cell

Expi293F cells (Thermo Fisher Scientific, Inc.) were seeded to a cell culture 6-well plate at 7.5×10⁶ cells/3 mL/well. Two types, heavy and light chain antibody expression units constructed in (3) were co-introduced into Expi293F cells using Expifectamine 293 Reagent (Thermo Fisher Scientific, Inc.). On day 5 after the introduction, a cell supernatant was collected and used to evaluate the binding properties of the produced antibodies.

### (5) Evaluation for Binding Property of Antibody Using Flow Cytometry

The human LPA1-stably expressing CHO cell or mouse LPA1-stably expressing CHO cell, and a negative control cell were washed with FACS buffer and suspended in FACS buffer at a cell concentration of 1×10⁷ cells/mL. To this suspension, Fc Block (Cytek Biosciences, Inc.) was added in an amount of 1/500 of the cell suspension, to block the cells at 4° C. for 30 minutes. After the blocking, the cells were suspended at 2×10⁵ cells/50 μL. This cell suspension was mixed with the cell supernatant collected in (4) and incubated at 4° C. for 1 hour. After the incubation, the cells were washed with 100 μL of FACS buffer twice. As a secondary antibody, a phycoerythrin-labeled anti-mouse IgG antibody (SouthernBiotech) dilution was added to wells at 50 μL/well and incubated at 4° C. for 1 hour. The cells were washed with 100 μL of FACS buffer twice and then suspended in 80 μL of FACS buffer, and a fluorescence intensity of the cell surface was measured using a flow cytometer iQue (Sartorius AG) to evaluate the binding property of the antibodies. An antibody unit that bound to the human LPA1-stably expressing CHO cell or mouse LPA1-stably expressing CHO cell and did not bind to the negative control cell was designated as a primary hit antibody.

### [Example 3] Screening of Antibody by Evaluating Inhibition of Intracellular cAMP Signaling

A purified antibody was prepared from a culture supernatant corresponding to the primary hit antibody in Example 2 by Protein A affinity purification according to an ordinary method and subjected to the following evaluation. A concentration of intracellular cyclic adenosine monophosphate (cAMP) was measured using LANCE Ultra cAMP Kit (PerkinElmer, Inc.).

The human LPA1-stably expressing CHO cell or mouse LPA1-stably expressing CHO cell was cultured in an FBS-free Ham's F-12 culture medium containing 0.5% of BSA, 100 units/mL of penicillin, and 100 μg/mL of streptomycin for 24 hours. The cells were collected from the cell culture dish by an enzyme-free cell dissociation buffer (Thermo Fisher Scientific, Inc.) and washed with Hanks' Balanced Salt Solution (HBSS). The cells were suspended at 2×10⁵ cells/mL in a stimulation buffer (HBSS containing 5 mM of HEPES, 0.5 mM of 3-isobutyl-1-methylxanthine (IBMX), and 0.1% of BSA), and dispensed into OptiPlate-96 well (PerkinElmer, Inc.) at 12.5 μL (2,500 cells)/well. The antibodies diluted to 400 nM in the stimulation buffer was added to the wells at 6.25 μL/well (final concentration: 100 nM) and allowed to stand for 15 minutes at room temperature. Subsequently, a mixture of 12 M Forskolin (final concentration: 3 μM) and 200 nM of oleoyl-L-α-lysophosphatidic acid sodium salt (Merck KGaA) (final concentration: 50 nM) was added to wells at 6.25 μL/well, and allowed to stand at room temperature for 30 minutes. An Eu-cAMP tracer solution and a ULight-anti-cAMP antibody solution, which had been diluted in a cAMP Detection Buffer appended to the kit according to the manufacturer's instruction, were added to the wells at 12.5 L/well respectively, and allowed to stand at room temperature for 1 hour. Then, a time-resolved fluorescence resonance energy transfer (TR-FRET) was measured using a plate reader EnVision 2104 (Perkin-Elmer, Inc.). For an inhibition rate (0%), a value measured in adding Forskolin and lysophosphatidic acid was standardized as 000 inhibition rate and a value measured in adding only Forskolin was standardized as 10000 inhibition rate, and the inhibition rate was calculated as a relative intracellular cAMP signaling inhibition rate (0).

Among (the primary hit antibodies, eleven types of antibodies: 210309-1-C, 210309-1-G, 210309-2-A, 210309-2-D, 210309-4-A, 210310-1-D, 210420-4-E, 210420-1-H, 210420-3-E, 21 1222-1-G, and 211222-1-A, which inhibited lysophosphatidic acid-induced intracellular cAMP signaling in the human LPA1-expressing cell and mouse LPA1-expressing cell, were selected as antibodies having the activity of blocking the LPA1-dependent cell functions.

Table 1 presents, for the eleven antibodies, antigen types from which the antibodies were obtained, and lysophosphatidic acid-induced intracellular cAMP signaling inhibition rates ofthe antibodies in the human and mouse LPA1-expressing cells.

TABLE 1

| | | Intracellular cAMP signaling inhibition | |
|---|---|---|---|
| | | (Inhibition rate %/100 nM) | |
| | | Human LPA1- | Mouse LPA1- |
| Antibody | Antigen | expressing cell | expressing cell |
| 210309-1-C | Human LPA1 | 47 | 20 |
| 210309-1-G | Human LPA1 | 34 | 36 |
| 210309-2-A | Human LPA1 | 32 | 32 |
| 210309-2-D | Human LPA1 | 51 | 53 |
| 210309-4-A | Human LPA1 | 95 | 50 |
| 210310-1-D | Human LPA1 | 22 | 25 |
| 210420-4-E | Mouse LPA1 | 24 | 46 |
| 210420-1-H | Mouse LPA1 | 22 | 37 |
| 210420-3-E | Mouse LPA1 | 44 | 50 |
| 211222-1-G | Human LPA1 | 22 | 49 |
| 211222-1-A | Human LPA1 | 1 | 30 |

### [Example 4] Screening of Antibody by Flow Cytometry Evaluation of Binding to Lysophosphatidic Acid Receptor

To evaluate the binding specificity of lysophosphatidic acid receptor subtypes to LPA1, LPA2, and LPA3, artificial genes added with a FLAG tag sequence at 5′ terminal were synthesized by using, as a base, human LPA1 (SEQ ID NO: 111), mouse LPA1 (SEQ ID NO: 112), as well as human LPA2 (NP_004711, SEQ ID NO: 119), mouse LPA2 (NP_064412, SEQ ID NO: 120), human LPA3 (NP_036284, SEQ ID NO: 121), and mouse LPA3 (NP_075359, SEQ ID NO: 122) which had been registered in GenBank. HEK293FT cell was transfected with 10 g of an expression vector carrying each synthetic gene using Lipofectamine 2000, to prepare a human LPA1 gene transfected cell, a mouse LPA1 gene transfected cell, a human LPA2 gene transfected cell, a mouse LPA2 gene transfected cell, a human LPA3 gene transfected cell, and a mouse LPA3 gene transfected cell.

The next day, the cells were washed with PBS, then detached from the cell culture dishes by an enzyme-free cell dissociation buffer (Thermo Fisher Scientific, Inc.), then washed with PBS, and then collected by centrifugation. Goat serum (Thermo Fisher Scientific, Inc.) was diluted by 2 times in FACS buffer (PBS containing 1% of FBS), into which the cells were suspended at 1×10⁷ cells/mL. This suspension was allowed to stand at 4° C. for 30 minutes to block the cells. The cells were collected by centrifugation and suspended in FACS buffer at 4×10⁶ cells/mL, and this suspension was dispensed into a V-bottom 96-well plate (Thermo Fisher Scientific, Inc.) at 25 μL (1×10⁵ cells)/well.

To the plate, 25 g/mL of eleven types of antibodies selected in Example 3 (210309-1-C, 210309-1-G, 210309-2-A, 210309-2-D, 210309-4-A, 210310-1-D, 210420-4-E, 210420-1-H, 210420-3-E, 211222-1-G, 211222-1-A), 20 g/mL of anti-FLAG mouse IgG antibody (Sigma-Aldrich Co. LLC) or mouse isotype control antibody (Fujifilm Wako Pure Chemicals Corporation) were added at 25 μL/well, and allowed to stand at 4° C. for 1 hour. The cells were collected by centrifugation and washed with 120 μL of FACS buffer. This washing operation was repeated twice. To the cell sediment, 25 μL each of Goat Anti-Mouse IgG H&L (DyLight 650) (Abcam) that was a secondary antibody diluted by 500 times in FACS buffer was dispensed, then suspended, and then allowed to stand at 4° C. for 1 hour. The cells were washed twice, then suspended in 50 μL of FACS buffer, and the whole content thereof was transferred to a V-bottom 96-well plate (Greiner), to measure the binding of antibodies to the cells using a flow cytometer (Agilent Novocyte).

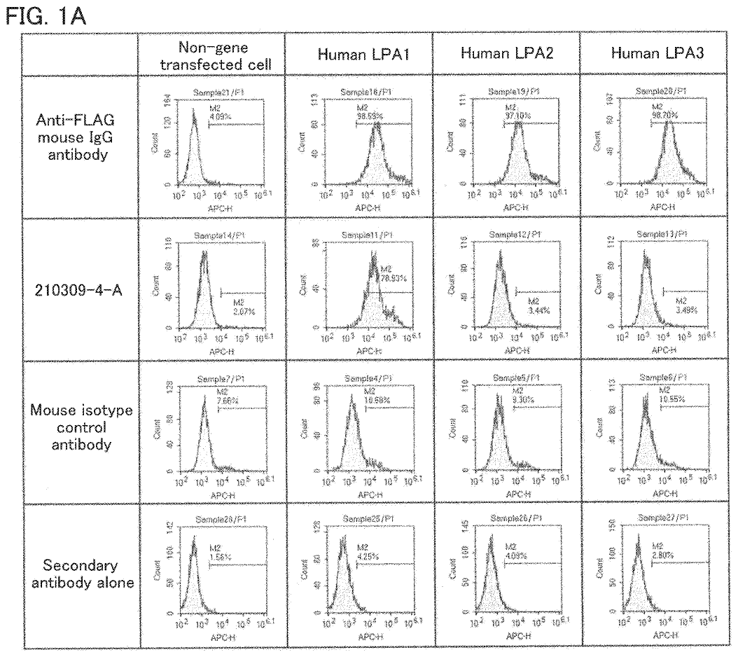

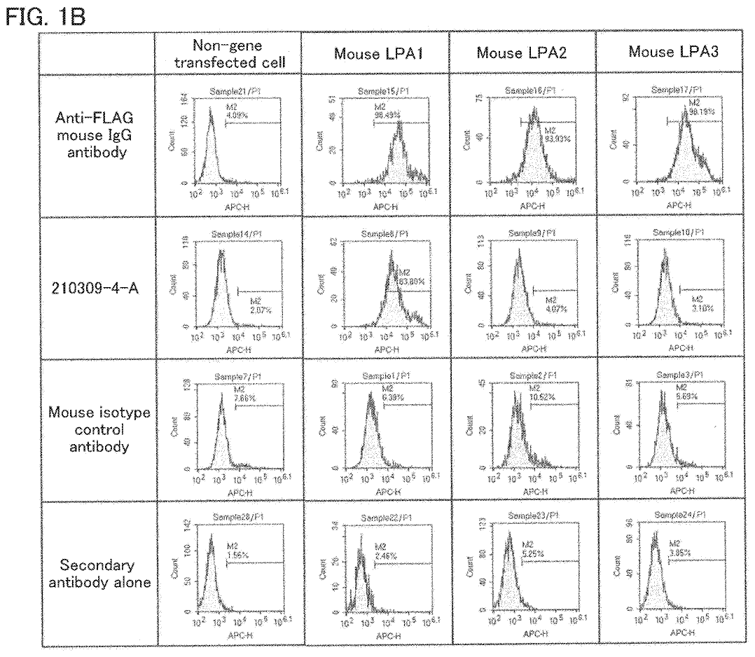

As representative examples, histograms of 210309-4-A measured by the flow cytometer are presented in FIG. 1. It was investigated whether or not a rightward shift of the histogram was caused in the case using the gene transfected cells compared to the case using the non-transfected cells. First, in the case using the anti-FLAG mouse IgG antibody as the primary antibody, all of the human LPA1 gene transfected cell, the human LPA2 gene transfected cell, the human LPA3 gene transfected cell, the mouse LPA1 gene transfected cell, the mouse LPA2 gene transfected cell, and the mouse LPA3 gene transfected cell showed the rightward shifts. On the other hand, in the case using the mouse isotype control antibody as the primary antibody or the case using the secondary antibody alone, all gene transfected cells showed no rightward shift. From this finding, it was confirmed that all receptors were expressed on the cell surface.

On the other hand, in the case using 210309-4-A as the primary antibody, only the human LPA1 gene transfected cell and mouse LPA1 gene transfected cell showed rightward shifts in the histograms. From this finding, it was confirmed that 210309-4-A bound specifically to the human LPA1 and mouse LPA extracellular domains but not to any of the human LPA2, human LPA3, mouse LPA2, and mouse LPA3 extracellular domains.

For antibodies other than 210309-4-A, the same results as in FIG. 1 were obtained.

The binding specificities of the eleven antibodies including antibodies other than 210309-4-A are presented in Table 2. Antibodies showing a rightward shift in the histogram were indicated as “+”, antibodies showing no rightward shift were indicated as “−”. It was observed that all the eleven antibodies bound specifically to the extracellular domains of the human LPA1 and mouse LPA but not to all extracellular domains of the human LPA2, human LPA3, mouse LPA2, and mouse LPA3, and the eleven antibodies were confirmed to bind specifically to the extracellular domain of the human LPA1 but not specifically to the extracellular domains of the human LPA2 and human LPA3.

TABLE 2

| | Binding specificity | | | | | |
|---|---|---|---|---|---|---|
| | Human | Human | Human | Mouse | Mouse | Mouse |
| Antibody | LPA1 | LPA2 | LPA3 | LPA1 | LPA2 | LPA3 |
| 210309-1-C | + | − | − | + | − | − |
| 210309-1-G | + | − | − | + | − | − |
| 210309-2-A | + | − | − | + | − | − |
| 210309-2-D | + | − | − | + | − | − |
| 210309-4-A | + | − | − | + | − | − |
| 210310-1-D | + | − | − | + | − | − |
| 210420-4-E | + | − | − | + | − | − |
| 210420-1-H | + | − | − | + | − | − |
| 210420-3-E | + | − | − | + | − | − |
| 211222-1-G | + | − | − | + | − | − |
| 211222-1-A | + | − | − | + | − | − |

### [Example 5] CDR Sequencing in Antibody Heavy Chain Variable Region and Antibody Light Chain Variable Region

The eleven antibody genes 210309-1-C, 210309-1-G, 210309-2-A, 210309-2-D, 210309-4-A, 210310-1-D, 210420-4-E, 210420-1-H, 210420-3-E, 211222-1-G, and 211222-1-A selected in Example 3 were cloned into pET vector by a homologous sequence-selective recombination cloning (Kurosawa N, Yoshioka M, and Isobe M., “Target-selective homologous recombination cloning for high-throughput generation of monoclonal antibodies from single plasma cells”, BMC Biotechnol., 2011; 11: 39). Furthermore, to analyze the sequence of the cloned antibody heavy chain variable region (VH region), the VH region was sequenced by a sequence analysis using the VH second reverse primer (SEQ ID NO: 117) used in Example 2. Similarly, to analyze the sequence of the cloned antibody light chain variable region (VL region), the VL region was sequenced by a sequence analysis using the VL second reverse primer (SEQ ID NO: 118) used in Example 2. For determination of the CDR, Kabat numbering method and IIMGT numbering method are combined for analysis, a region comprehensively including regions determined by the both numbering methods was determined as the CDR.

Amino acid sequences (AA) of the heavy chain variable region, the heavy chain CDRs 1 to 3, the light chain variable region, and the light chain CDRs 1 to 3 of each antibody, as well as the nucleotide sequences of DNAs individually coding for the heavy chain variable region and light chain variable region of each antibody are summarized in Tables 3-1 to 3-11.

TABLE 3-1
TABLE 3-1: 210309-4-A

| SEQ ID | | | |
|---|---|---|---|
| NO: | Region | Type | Sequence |
| 1 | Heavy chain CDR1 | AA | GYTFTSYGIS |
| 2 | Heavy chain CDR2 | AA | EIYPRSGNTYYNEKFKG |
| 3 | Heavy chain CDR3 | AA | ARESISRRLGWNFDV |
| 4 | Light chain CDR1 | AA | RASENIYSFLA |
| 5 | Light chain CDR2 | AA | NAKTLTE |
| 6 | Light chain CDR3 | AA | QHHYGPPLT |
| 7 | Heavy chain | AA | QVQLQQSGAELARPGASVKLSCKASGYTFTSYGISWVKQRTGQGLEWIGEIY |
| | variable region | | PRSGNTYYNEKFKGKATLTADKSSSTAYMELRSLTSEDSAVYFCARESISRR |
| | | | LGWNFDVWGTGTTVTVSS |
| 8 | Heavy chain | DNA | caggttcagctgcagcagtctggagctgagctggcgaggcctggggcttcag |
| | variable region | | tgaagctgtcctgcaaggcttctggctacaccttcacaagctatggtataag |
| | | | ctgggtgaagcagagaactggacagggccttgagtggattggagagatttat |
| | | | cctagaagtggtaatacttactacaatgagaagttcaagggcaaggccacac |
| | | | tgactgcagacaaatcctccagcacagcgtacatggagctccgcagcctgac |
| | | | atctgaggactctgcggtctatttctgtgcaagagagtctatatcacgacgg |
| | | | ctaggctggaacttcgatgtctggggcacagggaccacggtcaccgtctcct |
| | | | ca |
| 9 | Light chain | AA | DIHMTQSPASLSASVGETVTITCRASENIYSFLAWYQQKQGKSPHLVVYNAK |
| | variable region | | TLTEGVPSRFSGSGSGTHFSLKINSLQPEDFGIYYCQHHYGPPLTFGAGTKL |
| | | | ELK |
| 10 | Light chain | DNA | gacatccacatgactcagtctccagcctccctatctgcatctgtgggagaaa |
| | variable region | | ctgtcaccatcacatgtcgagcaagtgagaatatttacagttttttagcatg |
| | | | gtatcagcagaaacagggaaaatctcctcacctcgtggtctacaatgcaaag |
| | | | accttaacagaaggtgtgccatcaaggttcagtggcagtggatcaggcacac |
| | | | acttttctctgaagatcaacagccttcagcctgaagattttgggatttatta |
| | | | ctgtcaacatcattatggtcctcctctcacgttcggtgctgggaccaagctg |
| | | | gagctgaaa |

TABLE 3-2
TABLE 3-2: 210309-1-C

| SEQ ID | | | |
|---|---|---|---|
| NO: | Region | Type | Sequence |
| 11 | Heavy chain CDR1 | AA | GYSFTAYGIS |
| 12 | Heavy chain CDR2 | AA | EIYPRSGNTYYSEKFKG |
| 13 | Heavy chain CDR3 | AA | ARESLLKRLGWYFDV |
| 14 | Light chain CDR1 | AA | RASQNIYSFLA |
| 15 | Light chain CDR2 | AA | NAKTLAE |
| 16 | Light chain CDR3 | AA | QHHYGPPLT |
| 17 | Heavy chain | AA | QVHLQQSGAELARPGASVKLSCKASGYSFTAYGISWVQQRTGQGLEWIGEIY |
| | variable region | | PRSGNTYYSEKFKGKATLTADKSSSTAYMELRSLTSEDSAVYFCARESLLKR |
| | | | LGWYFDVWGTGTTVTVSS |
| 18 | Heavy chain | DNA | caggttcatctgcagcagtctggagctgagctggcgaggcctggggcttcag |
| | variable region | | tgaagctgtcctgcaaggcttctggctacagcttcacagcctatggtataag |
| | | | ctgggtgcagcagagaactggacagggccttgagtggattggagagatttat |
| | | | ccaagaagtggtaatacttactacagtgagaagttcaagggcaaggccacac |
| | | | tgactgcagacaaatcctccagtacagcgtacatggagctccgcagcctgac |
| | | | atctgaggactctgcggtctatttctgtgcaagagagtctctattaaaacgg |
| | | | ctaggctggtacttcgatgtctggggcacagggaccacggtcaccgtctcct |
| | | | ca |
| 19 | Light chain | AA | DIQMTQSPASLSASVGETVTITCRASQNIYSFLAWYQQKQGNSPQLLVYNAK |
| | variable region | | TLAEGVPSRFSGSGSGTQFSLKINSLQPEDFGSYYCQHHYGPPLTFGVGTKL |
| | | | ELK |
| 20 | Light chain | DNA | gacatccagatgactcagtctccagcctccctatctgcatctgtgggagaaa |
| | variable region | | ctgtcaccatcacatgtcgagcaagtcagaatatttacagttttttagcatg |
| | | | gtatcagcagaaacagggaaattctcctcagctcctggtctataatgcaaaa |
| | | | accttagcagaaggtgtgccatcaaggttcagtggcagtggatcaggcacac |
| | | | agttttctctgaagatcaacagcctgcagcctgaagattttgggagttatta |
| | | | ctgtcaacatcattatggtcctcctctcacgttcggtgttgggaccaagctg |
| | | | gagctgaaa |

TABLE 3-3
TABLE 3-3: 210309-1-G

| SEQ ID | | | |
|---|---|---|---|
| NO: | Region | Type | Sequence |
| 21 | Heavy chain CDR1 | AA | GYTFTSYGIS |
| 22 | Heavy chain CDR2 | AA | EIYPRSGVTYYNEKFKG |
| 23 | Heavy chain CDR3 | AA | ARESISLRLGWYFDV |
| 24 | Light chain CDR1 | AA | RASENIYRFLA |
| 25 | Light chain CDR2 | AA | NAKTLAE |
| 26 | Light chain CDR3 | AA | QHHYGPPLT |
| 27 | Heavy chain | AA | QVQLQQSGAELARPGASMKLSCRASGYTFTSYGISWVKQRTGQGLEWIGEIY |
| | variable region | | PRSGVTYYNEKFKGRATLTADKSSSTAFMELRSLTSEDSAVYFCARESISLR |
| | | | LGWYFDVWGTGTTVTVSS |
| 28 | Heavy chain | DNA | caggttcagctgcagcagtctggagctgagctggcgaggcctggggcttcaa |
| | variable region | | tgaagctgtcctgcagggcttctggctacaccttcacaagctatggtataag |
| | | | ctgggtgaagcagagaactggacagggccttgagtggattggagagatttat |
| | | | cccagaagtggtgttacttactacaatgagaagttcaagggtagggccacac |
| | | | tgactgcagacaaatcctccagcacagcgttcatggagctccgcagcctgac |
| | | | atctgaggactctgcggtctatttctgtgcaagagagtctatatcactacga |
| | | | ctaggctggtacttcgatgtctggggcacagggaccacggtcaccgtctcct |
| | | | ca |
| 29 | Light chain | AA | DIQMTQSPASLSASVGDTVTITCRASENIYRFLAWYQQKQRKSPHLLVYNAK |
| | variable region | | TLAEGVPSRFSGSGSGTQFSLKINSLQPEDFGSYYCQHHYGPPLTFGAGTKL |
| | | | ELT |
| 30 | Light chain | DNA | gacatccagatgactcagtctccagcctccctatctgcatctgtgggagata |
| | variable region | | ctgtcaccatcacatgtcgagcaagtgagaatatttacaggtttttagcatg |
| | | | gtatcagcagaaacagagaaaatctccgcacctcctggtctataatgcaaaa |
| | | | accttagcagaaggtgtgccatcaaggttcagtggcagtggatcaggcacac |
| | | | agttttctctgaagatcaacagtctgcagcctgaagattttgggagttatta |
| | | | ttgtcaacatcattatggtcctccactcacgttcggtgctgggaccaagctg |
| | | | gagctgaca |

TABLE 3-4
TABLE 3-4: 210309-2-A

| SEQ ID | | | |
|---|---|---|---|
| NO: | Region | Type | Sequence |
| 31 | Heavy chain CDR1 | AA | GYTFTSYGIS |
| 32 | Heavy chain CDR2 | AA | EIYPRSGNTYYNEKFKG |
| 33 | Heavy chain CDR3 | AA | ARESILLRLGWYFDV |
| 34 | Light chain CDR1 | AA | RASENIYRFLA |
| 35 | Light chain CDR2 | AA | NAKTLAE |
| 36 | Light chain CDR3 | AA | QHHYGPPLT |
| 37 | Heavy chain | AA | QVQLQQSGAELARPGASVKLSCKASGYTFTSYGISWVKQRTGQGLEWIGEIY |
| | variable region | | PRSGNTYYNEKFKGRATLTADKSSSTAYMELRSLTSEDSAVYFCARESILLR |
| | | | LGWYFDVWGTGTTVTVSS |
| 38 | Heavy chain | DNA | caggttcagctgcagcagtctggagctgagctggcgaggcctggggcttcag |
| | variable region | | tgaagctgtcctgcaaggcttctggctacaccttcacaagctatggtataag |
| | | | ctgggtgaagcagagaactggacagggccttgagtggattggagagatttat |
| | | | cctagaagtggtaatacttactacaatgagaagttcaagggcagggccacac |
| | | | tgactgcagacaaatcctccagcacagcgtacatggagctccgcagcctgac |
| | | | atctgaggactctgcggtctatttctgtgcaagagagtctatattactacgg |
| | | | ctaggctggtacttcgatgtctggggcacagggaccacggtcaccgtctcct |
| | | | ca |
| 39 | Light chain | AA | DIQMTQSPASLSASVGGTVTITCRASENIYRFLAWYQQKQGKSPQLLVYNAK |
| | variable region | | TLAEGVPSRFSGSGSGTQFSLKINSLQPEDFGIYYCQHHYGPPLTFGAGTKL |
| | | | ELK |
| 40 | Light chain | DNA | gacatccagatgactcagtctccagcctccctatctgcatctgtgggaggaa |
| | variable region | | ctgtcaccatcacatgtcgagcaagtgagaatatttacaggtttttagcatg |
| | | | gtatcagcagaaacagggaaaatctcctcagctcctggtctataatgcaaaa |
| | | | accttagcagaaggtgtgccatcaaggttcagtggcagtggatcaggcacac |
| | | | agttttctctgaagatcaacagccttcagcctgaagattttgggatttatta |
| | | | ctgtcaacatcattatggtcctcctctcacgttcggtgctgggaccaagctg |
| | | | gagctgaaa |

TABLE 3-5
TABLE 3-5: 210309-2-D

| SEQ ID | | | |
|---|---|---|---|
| NO: | Region | Type | Sequence |
| 41 | Heavy chain CDR1 | AA | GYTFTSYGIS |
| 42 | Heavy chain CDR2 | AA | EIYPRSGNTYYNEKVKG |
| 43 | Heavy chain CDR3 | AA | ARESIFLRLGWYFDV |
| 44 | Light chain CDR1 | AA | RASDNIYRFLA |
| 45 | Light chain CDR2 | AA | NAKTLAE |
| 46 | Light chain CDR3 | AA | QHHYGPPLT |
| 47 | Heavy chain | AA | QVQLQQSGAELARPGASVKLSCKASGYTFTSYGISWVKQRTGQGLEWIGEIY |
| | variable region | | PRSGNTYYNEKVKGKATLTADKSSSTAYMELRSLTSEDSAVYFCARESIFLR |
| | | | LGWYFDVWGTGTTVTVSS |
| 48 | Heavy chain | DNA | caggttcagctgcagcagtctggagctgagctggcgaggcctggggcttcag |
| | variable region | | tgaagctgtcctgcaaggcttctggctacaccttcacaagctatggtataag |
| | | | ttgggtgaagcagagaactggacagggccttgagtggattggagagatttat |
| | | | cctagaagtggtaatacttactacaatgagaaggtcaagggcaaggccacac |
| | | | tgactgcagacaaatcctccagcacagcgtacatggagctccgcagcctgac |
| | | | atctgaggactctgcggtctatttctgtgcaagagagtctatattcctacgt |
| | | | ctaggctggtacttcgatgtctggggcacagggaccacggtcaccgtctcct |
| | | | ca |
| 49 | Light chain | AA | DIQMTQSPASLSASVGETVTITCRASDNIYRFLAWYQQKQGKSPQLLVYNAK |
| | variable region | | TLAEGVPSRFSGSGTGTQFSLKINSLQPEDFGIYYCQHHYGPPLTFGAGTKL |
| | | | ELK |
| 50 | Light chain | DNA | gacatccagatgactcagtctccagcctccctatctgcatctgtgggagaaa |
| | variable region | | ctgtcaccatcacatgtcgagcaagtgacaatatttacaggtttttagcatg |
| | | | gtatcagcagaaacagggaaaatctcctcagctcctggtctataatgcaaaa |
| | | | accttagcagaaggtgtgccatcaaggttcagtggcagtggaacaggcacac |
| | | | agttttctctgaagatcaacagtcttcagcctgaagattttgggatttatta |
| | | | ctgtcaacatcattatggtcctcctctcacgttcggtgctgggaccaagctg |
| | | | gagctgaaa |

TABLE 3-6
TABLE 3-6: 210310-1-D

| SEQ ID | | | |
|---|---|---|---|
| NO: | Region | Type | Sequence |
| 51 | Heavy chain CDR1 | AA | GYTFTSFGVN |
| 52 | Heavy chain CDR2 | AA | QIYPRTGTTYHNERFKG |
| 53 | Heavy chain CDR3 | AA | AREGDRYSLAY |
| 54 | Light chain CDR1 | AA | RASENIYRFLA |
| 55 | Light chain CDR2 | AA | NAKTLVE |
| 56 | Light chain CDR3 | AA | QHHYGIPLT |
| 57 | Heavy chain | AA | QVQVQQSGAELARPGASVRLSCKASGYTFTSFGVNWVKQRTGQGLEWIGQIY |
| | variable region | | PRTGTTYHNERFKGKATLTTDKSSSTAYMELRSLTSEDSAVYFCAREGDRYS |
| | | | LAYWGQGTLVTVSA |
| 58 | Heavy chain | DNA | caggttcaggtacagcagtccggagctgagctggcgaggcctggggcatcag |
| | variable region | | tgaggctgtcctgcaaggcttctggctacaccttcacaagctttggtgtaaa |
| | | | ctgggtgaagcagagaactggacagggccttgagtggattggacagatttat |
| | | | cctagaactggtactacttaccacaatgagaggttcaagggcaaggccacac |
| | | | tgactacagacaaatcctccagcacagcgtacatggagctccgcagcctgac |
| | | | atctgaggactctgcggtctatttctgtgcacgagagggagataggtactca |
| | | | cttgcttactggggccaagggactctggtcactgtctctgca |
| 59 | Light chain | AA | DIQMTQSPASLSASVGETVTITCRASENIYRFLAWYQQKQGKSPQLLVYNAK |
| | variable region | | TLVEGVPSRFSGSGSGTQFSLKINNLQPEDFGSYYCQHHYGIPLTFGAGTKL |
| | | | ELK |
| 60 | Light chain | DNA | gacatccagatgactcagtctccagcctccctatctgcatctgtgggagaaa |
| | variable region | | ctgtcaccatcacatgtcgagcaagtgagaatatttacaggtttttagcatg |
| | | | gtatcagcagaaacagggaaaatctcctcagctcctggtctataatgcaaaa |
| | | | accttagtagaaggtgtgccatcaaggttcagtggcagtggatcaggcacac |
| | | | agttttctctgaagatcaacaacctacagcctgaagattttgggagttatta |
| | | | ctgtcaacatcattatggtattccgctcacgttcggtgctgggaccaagctg |
| | | | gagctgaaa |

TABLE 3-7
TABLE 3-7: 210420-4-E

| SEQ ID | | | |
|---|---|---|---|
| NO: | Region | Type | Sequence |
| 61 | Heavy chain CDR1 | AA | GYTFASFGIS |
| 62 | Heavy chain CDR2 | AA | EIYPRSGTTYFNEKFRG |
| 63 | Heavy chain CDR3 | AA | AREGVRRYAMDY |
| 64 | Light chain CDR1 | AA | RASGNIYSFLA |
| 65 | Light chain CDR2 | AA | NAKTLAE |
| 66 | Light chain CDR3 | AA | QHHYGIPLT |
| 67 | Heavy chain | AA | QLHLQQSGAELARPGASVRLSCKASGYTFASFGISWVKQRTGQGLEYIGEIY |
| | variable region | | PRSGTTYFNEKFRGKATLSADKSSSTAYMELRSLTSEDSAVYFCAREGVRRY |
| | | | AMDYWGQGTSVTVSS |
| 68 | Heavy chain | DNA | cagcttcacctgcagcagtctggagctgagctggcgaggcctggggcttcag |
| | variable region | | tgaggctgtcctgcaaggcttctggctacaccttcgcaagttttggtataag |
| | | | ctgggtgaagcagagaactggacagggccttgaatacattggagagatttat |
| | | | cctagaagtggtactacttacttcaatgagaagttcaggggcaaggccacac |
| | | | tgagtgcagacaaatcatccagcacagcgtacatggagctccgcagcctgac |
| | | | atctgaggactctgcggtctatttctgtgcaagagagggagtacgacggtat |
| | | | gctatggactactggggtcaaggaacctcagtcaccgtctcctca |
| 69 | Light chain | AA | DIQVTQSPASLSASVGETVTITCRASGNIYSFLAWYQQKQGKSPQLLVYNAK |
| | variable region | | TLAEGVPSRFSGSGSGTQFSLKISSLQPEDFGSYYCQHHYGIPLTFGAGTKL |
| | | | DLK |
| 70 | Light chain | DNA | gacatccaggtgactcagtctccagcctccctatctgcgtctgtgggagaaa |
| | variable region | | ctgtcaccatcacatgtcgcgcaagtgggaatatttacagttttttagcatg |
| | | | gtatcagcagaaacagggaaaatctcctcaactcctggtctataatgcaaaa |
| | | | actttagcagaaggtgtgccatcaaggttcagtggcagtggatcaggcacac |
| | | | agttttctctgaagatcagcagcctgcagcctgaagattttgggagttatta |
| | | | ctgtcaacatcattatggtattcctctcacgttcggtgctgggaccaagttg |
| | | | gacctgaaa |

TABLE 3-8
TABLE 3-8: 210420-1-H

| SEQ ID | | | |
|---|---|---|---|
| NO: | Region | Type | Sequence |
| 71 | Heavy chain CDR1 | AA | GYTFASFGIS |
| 72 | Heavy chain CDR2 | AA | EIYPRSGTTYFNEKFRG |
| 73 | Heavy chain CDR3 | AA | AREGVRRYAMDY |
| 74 | Light chain CDR1 | AA | RASGNIYSFLA |
| 75 | Light chain CDR2 | AA | NAKTLAE |
| 76 | Light chain CDR3 | AA | QHHYGIPLT |
| 77 | Heavy chain | AA | QFQLQQSGAELARPGASVKLSCKASGYTFASFGISWVKQRTGQGLEYIGEIY |
| | variable region | | PRSGTTYFNEKFRGKATLTADKSSSTAYMELRSLTSEDSAVYFCAREGVRRY |
| | | | AMDYWGQGTSVTVSS |
| 78 | Heavy chain | DNA | cagtttcagctgcagcagtctggagctgagctggcgaggcctggggcttcag |
| | variable region | | tgaagctgtcctgcaaggcttctggctacaccttcgcaagctttggtataag |
| | | | ctgggtgaaacagagaactggacagggccttgaatacattggagagatttat |
| | | | cctagaagtggtactacttacttcaatgagaaattcaggggcaaggccacac |
| | | | tgactgcagacaaatcatccagcacagcgtacatggagctccgcagcctgac |
| | | | atctgaggactctgcggtctatttctgtgcaagagagggggtacgacggtat |
| | | | gctatggactactggggtcaaggaacctcagtcaccgtctcctca |
| 79 | Light chain | AA | DIQVTQSPASLSASVGETVTITCRASGNIYSFLAWYQQKQGKSPQLLVYNAK |
| | variable region | | TLAEGVPSRFSGSGSGTQFSLKISSLQPEDFGSYYCQHHYGIPLTFGGGTKL |
| | | | DLK |
| 80 | Light chain | DNA | gacatccaggtgactcagtctccagcctccctatctgcgtctgtgggagaaa |
| | variable region | | ctgtcaccatcacatgtcgcgcaagtgggaatatttacagttttttagcatg |
| | | | gtatcagcagaaacagggaaaatctcctcagctcctggtctataatgcaaaa |
| | | | accttagcagaaggtgtgccatcaaggttcagtggcagtggatcaggcacac |
| | | | agttttctctgaagattagcagcctgcagcctgaagattttgggagttatta |
| | | | ctgtcaacatcattatggtattcctctcacgttcggtggtgggaccaagttg |
| | | | gacctgaaa |

TABLE 3-9
TABLE 3-9: 210420-3-E

| SEQ ID | | | |
|---|---|---|---|
| NO: | Region | Type | Sequence |
| 81 | Heavy chain CDR1 | AA | GYTFASFGIS |
| 82 | Heavy chain CDR2 | AA | EIYPRSGTTYFNEKFRG |
| 83 | Heavy chain CDR3 | AA | AREGVRRYAMDY |
| 84 | Light chain CDR1 | AA | RASGNIYSFLA |
| 85 | Light chain CDR2 | AA | NAKTLAE |
| 86 | Light chain CDR3 | AA | QHHYGIPLT |
| 87 | Heavy chain | AA | QLQLQQSGAELARPGASVKLSCKASGYTFASFGISWVKQRAGQGLEYIGEIY |
| | variable region | | PRSGTTYFNEKFRGKATLTADKSSSTAYMELRRLTSEDSAVYFCAREGVRRY |
| | | | AMDYWGQGTSVTVSS |
| 88 | Heavy chain | DNA | cagcttcagctgcagcagtctggagctgagctggcgaggcctggggcttcag |
| | variable region | | tgaagctgtcctgcaaggcttctggctacaccttcgcaagttttggtataag |
| | | | ttgggtgaagcagagagctggacagggccttgagtacattggagagatttat |
| | | | cctagaagtggtactacttacttcaatgagaagttcaggggcaaggccacac |
| | | | tgactgcagacaaatcatccagcacagcgtacatggagctccgcagactgac |
| | | | atctgaggactctgcggtctatttctgtgcaagagagggggtacgacggtat |
| | | | gctatggactactggggtcaaggaacctcagtcaccgtctcctca |
| 89 | Light chain | AA | DIQVTQSPASLSASVGETVTITCRASGNIYSFLAWYQQKQGKSPQLLVYNAK |
| | variable region | | TLAEGVPSRFSGSGSGTQFSLKINSLQPEDFGSYYCQHHYGIPLTFGAGTKL |
| | | | DLK |
| 90 | Light chain | DNA | gacatccaggtgactcagtctccagcctccctatctgcgtctgtgggagaaa |
| | variable region | | ctgtcaccatcacatgtcgcgcaagtgggaatatttacagttttttagcatg |
| | | | gtatcagcagaaacagggaaaatctcctcagctcctggtctataatgcaaaa |
| | | | accttagcagaaggtgtgccatcaaggttcagtggcagtggatcaggcacac |
| | | | agttttctctgaagatcaacagcctgcagcctgaagattttgggagttatta |
| | | | ctgtcaacatcattatggtattcctctcacgttcggtgctgggaccaagttg |
| | | | gacctgaaa |

TABLE 3-10
TABLE 3-10: 211222-1-G

| SEQ ID | | | |
|---|---|---|---|
| NO: | Region | Type | Sequence |
| 91 | Heavy chain CDR1 | AA | GYTFTSFGVN |
| 92 | Heavy chain CDR2 | AA | QIYPRTGTTYHNERFKG |
| 93 | Heavy chain CDR3 | AA | AREGDRYSLAY |
| 94 | Light chain CDR1 | AA | RASENIYRFLA |
| 95 | Light chain CDR2 | AA | NAKTLVE |
| 96 | Light chain CDR3 | AA | QHHYGIPLT |
| 97 | Heavy chain | AA | QVQVQQSGAELARPGASVRLSCKASGYTFTSFGVNWVKQRTGQGLEWIGQIY |
| | variable region | | PRTGTTYHNERFKGKATLTADKSSSTAYMELRSLTSEDSAVYFCAREGDRYS |
| | | | LAYWGQGTLVTVSA |
| 98 | Heavy chain | DNA | caggttcaggtacagcagtccggagctgagctggcgaggcctggggcatcag |
| | variable region | | tgaggctgtcctgcaaggcttctggctacaccttcacaagttttggtgtaaa |
| | | | ctgggtgaagcagagaactggacagggccttgagtggattggacagatttat |
| | | | cctagaactggtactacttaccacaatgagaggttcaagggcaaggccacac |
| | | | tgactgcagacaagtcctccagcacagcgtacatggagctccgcagcctgac |
| | | | atctgaggactctgcggtctatttctgtgcacgagagggagataggtactca |
| | | | cttgcttactggggccaagggactctggtcactgtctctgca |
| 99 | Light chain | AA | DIQMTQSPASLSASVGETVTITCRASENIYRFLAWYQQKQGKSPQLLVYNAK |
| | variable region | | TLVEGVPSRFSGSGSGTQFSLKINNLQPEDFGSYYCQHHYGIPLTFGAGTKL |
| | | | ELK |
| 100 | Light chain | DNA | gacatccagatgactcagtctccagcctccctatctgcatctgtgggagaaa |
| | variable region | | ctgtcaccatcacatgtcgagcaagtgagaatatttacaggtttttagcatg |
| | | | gtatcagcagaaacagggaaaatctcctcagctcctggtctataatgcaaaa |
| | | | accttagtagaaggtgtgccatcaaggttcagtggcagtggatcaggcacac |
| | | | agttttctctgaagatcaacaacctacagcctgaagattttgggagttatta |
| | | | ctgtcaacatcattatggtattccgctcacgttcggtgctgggaccaagctg |
| | | | gagctgaaa |

TABLE 3-11
TABLE 3-11: 211222-1-A

| SEQ ID | | | |
|---|---|---|---|
| NO: | Region | Type | Sequence |
| 101 | Heavy chain CDR1 | AA | GYTFTSFGVN |
| 102 | Heavy chain CDR2 | AA | QIYPRSGTTYHNERFKG |
| 103 | Heavy chain CDR3 | AA | AREGDRYSLAY |
| 104 | Light chain CDR1 | AA | RASENIYRFLA |
| 105 | Light chain CDR2 | AA | NAKTLAE |
| 106 | Light chain CDR3 | AA | QHHYGIPLT |
| 107 | Heavy chain | AA | QVQVHQSGAELARPGASVRLSCKASGYTFTSFGVNWVKQRTGQGLEWIGQIY |
| | variable region | | PRSGTTYHNERFKGKATLTADKSSSTAYMELRSLTSEDSAVYFCAREGDRYS |
| | | | LAYWGQGTLVTVSA |
| 108 | Heavy chain | DNA | caggttcaggtacaccagtccggagctgagctggcgaggcctggggcatcag |
| | variable region | | tgaggctgtcctgcaaggcttctggctacaccttcacgagctttggtgtaaa |
| | | | ctgggtgaagcagagaactggacagggccttgagtggattggacagatttat |
| | | | cctagaagtggtactacttaccacaatgagaggttcaagggcaaggccacac |
| | | | tgactgcagacaaatcctccagcacagcgtacatggagctccgcagcctgac |
| | | | atctgaggactctgcggtctatttctgtgcaagagagggagataggtactca |
| | | | cttgcttactggggccaagggactctggtcactgtctctgca |
| 109 | Light chain | AA | DIQMTQSPASLSASVGETVTITCRASENIYRFLAWYQQKQGKSPQLLVSNAK |
| | variable region | | TLAEGVPSRFSGSGSGTQFSLKINYLQPEDFGNYYCQHHYGIPLTFGAGTKL |
| | | | ELK |
| 110 | Light chain | DNA | gacatccagatgactcagtctccagcctccctatctgcatctgtgggagaaa |
| | variable region | | ctgtcaccatcacatgtcgagcaagtgagaatatttacaggtttttagcatg |
| | | | gtatcagcagaaacagggaaaatctcctcagctcctggtctctaatgcaaaa |
| | | | accttagcagaaggtgtgccatcaaggttcagtggcagtggatcaggcacac |
| | | | agttttctctgaagatcaactacctacagcctgaagattttgggaattatta |
| | | | ctgtcaacatcattatggtattccgctcacgttcggtgctgggaccaagctg |
| | | | gagctgaaa |

### [Example 6] Evaluation of Binding Property to LPA1 by Flow Cytometry

An artificial gene was synthesized, in which a heavy chain constant region and a K chain constant region of mouse IgG1 were connected to the VH regions and VL regions of 210309-1-C, 210309-1-G, 210309-2-A, 210309-2-D, 210309-4-A, 210310-1-D, 210420-4-E, 210420-1-H, 210420-3-E, 21 1222-1-G, and 211222-1-A, determined in Example 5. The antibody genes were introduced into a CHO cell, and from the culture supernatant of the cell, purified antibodies were produced by affinity chromatography and subjected to the following test. As a result of the purity test by sodium dodecyl sulfate polyacrylamide gel electrophoresis (SDS-PAGE), all the purified antibodies were confirmed to have purities of 9000 or higher.

The human LPA1-stably expressing CHO cell and the mouse LPA1-stably expressing CHO cell were washed with PBS, then detached from the cell culture dishes by an enzyme-free cell dissociation buffer (Thermo Fisher Scientific, Inc.), then washed with PBS, and then collected by centrifugation. The human LPA1-stably expressing CHO cell and the mouse LPA1-stably expressing CHO cell were suspended at 1×10⁷ cells/mL in Fc Block (Sci-Tech Biosciences, Inc.) diluted by 500 times in FACS buffer, and then allowed to stand at 4° C. for 30 minutes to block the cells. The cells were collected by centrifugation and suspended in FACS buffer at 4×10⁶ cells/mL, and this suspension was dispensed into a V-bottom 96-well plate (Thermo Fisher Scientific, Inc.) at 25 μL (1×10⁵ cells)/well. As primary antibodies, the eleven types of LPA1 antibodies produced and purified by the method described above and a mouse isotype control antibody (Fujifilm Wako Pure Chemicals Corporation) were diluted stepwise, which was added to each well at 25 μL/well, and allowed to stand at 4° C. for 1 hour. The cells were collected by centrifugation and washed with 120 μL of FACS buffer. This washing operation was repeated twice. To the cell sediment, 25 μL each of Goat Anti-Mouse IgG H&L (DyLight 650) (Abcam) that was a secondary antibody diluted by 500 times in FACS buffer was dispensed, then suspended, and then allowed to stand at 4° C. for 1 hour. The cells were washed twice, then suspended in 50 μL of FACS buffer, and the binding of antibodies to the cells was measured using a flow cytometer (Agilent Novocyte).

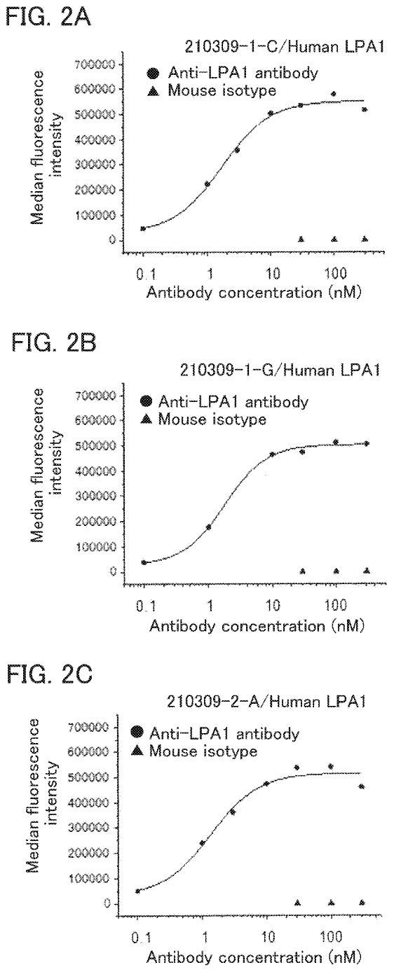

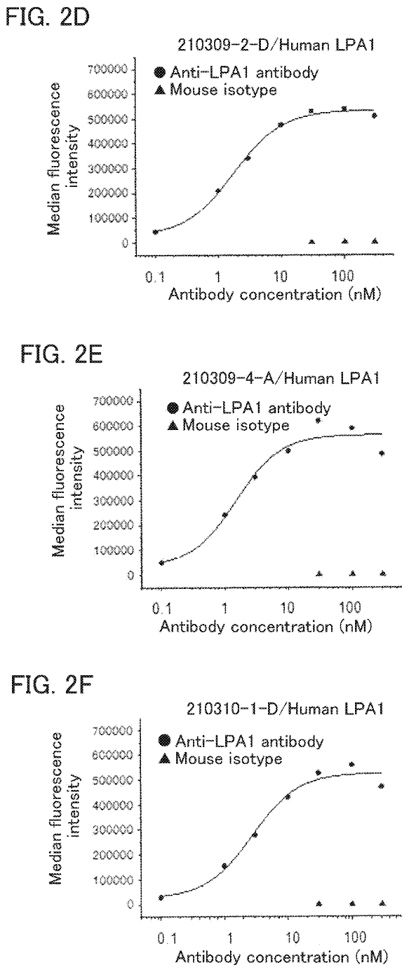

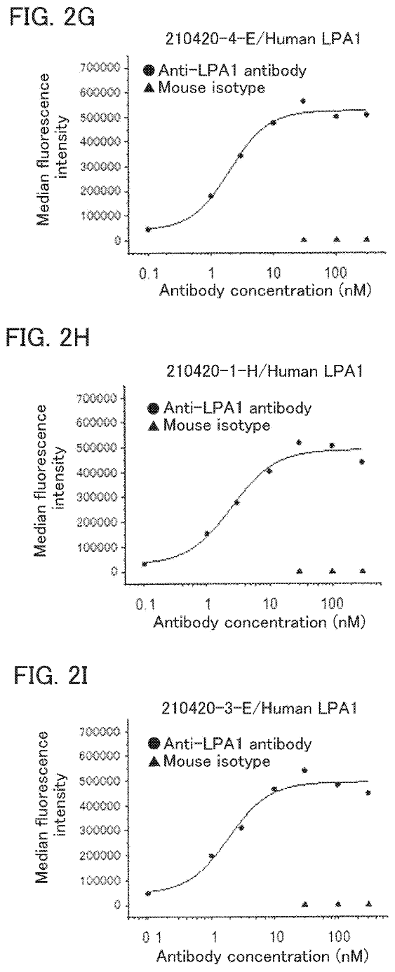

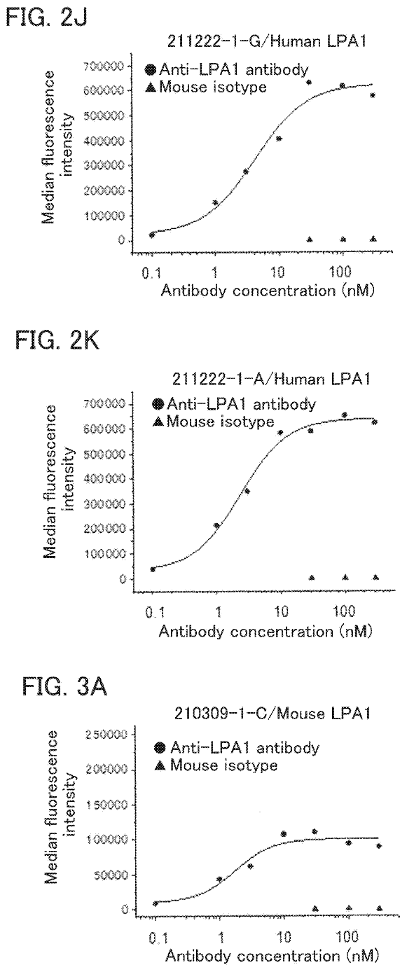

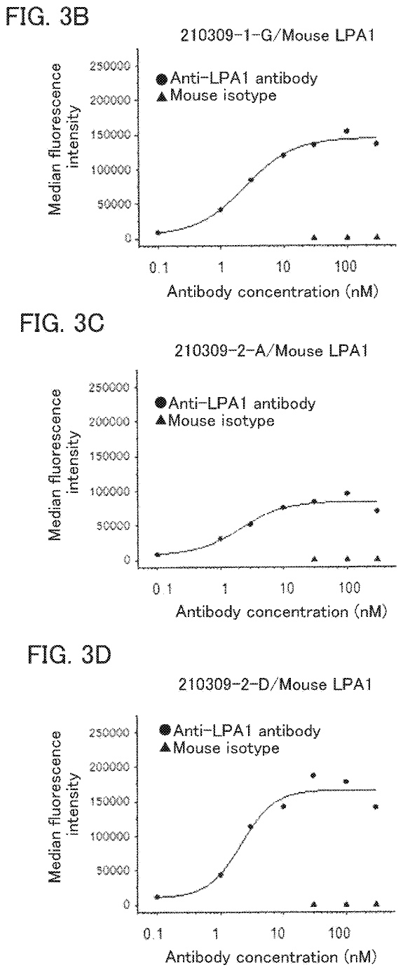

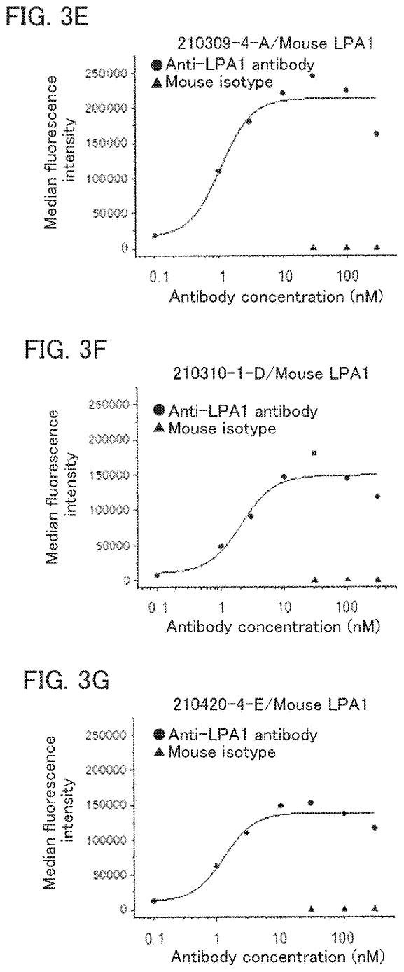

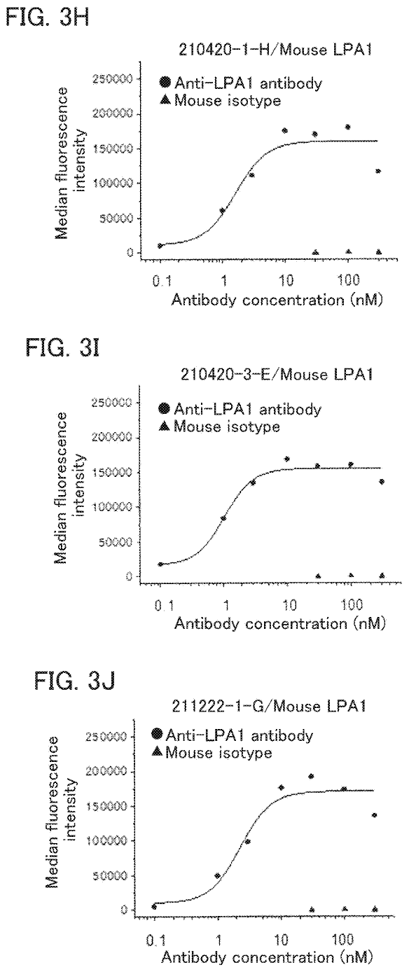

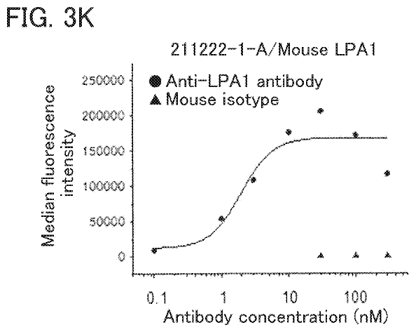

A graph was created, in which a concentration of the antibody was plotted on the vertical axis, and a median fluorescence intensity of the flow cytometer histogram was plotted on the horizontal axis. A11 of the eleven antibodies were confirmed to dose-dependently bind to the human LPA1-stably expressing CHO cell and the mouse LPA1-stably expressing CHO cell. The results of the binding of each antibody to the human LPA1-stably expressing CHO cell are presented in FIG. 2A to FIG. 2K, and the results of the binding of each antibody to the mouse LPA1-stably expressing CHO cell are presented in FIG. 3A to FIG. 3K. A 50% binding concentration (50% effective concentration (EC50)) of each antibody was calculated and summarized in Table 4.

TABLE 4

Binding to LPA1-stably expressing cell
EC50 (nM)

| | Human LPA1-stably | Mouse LPA1-stably |
|---|---|---|
| Antibody | expressing cell | expressing cell |
| 210309-1-C | 1.7 | 1.8 |
| 210309-1-G | 1.8 | 2.4 |
| 210309-2-A | 1.4 | 2.1 |
| 210309-2-D | 1.8 | 2.1 |
| 210309-4-A | 1.5 | 1.1 |
| 210310-1-D | 2.7 | 2.1 |
| 210420-4-E | 2.0 | 1.3 |
| 210420-1-H | 2.5 | 1.6 |
| 210420-3-E | 1.9 | 1.0 |
| 211222-1-G | 4.2 | 2.3 |
| 211222-1-A | 2.3 | 2.0 |

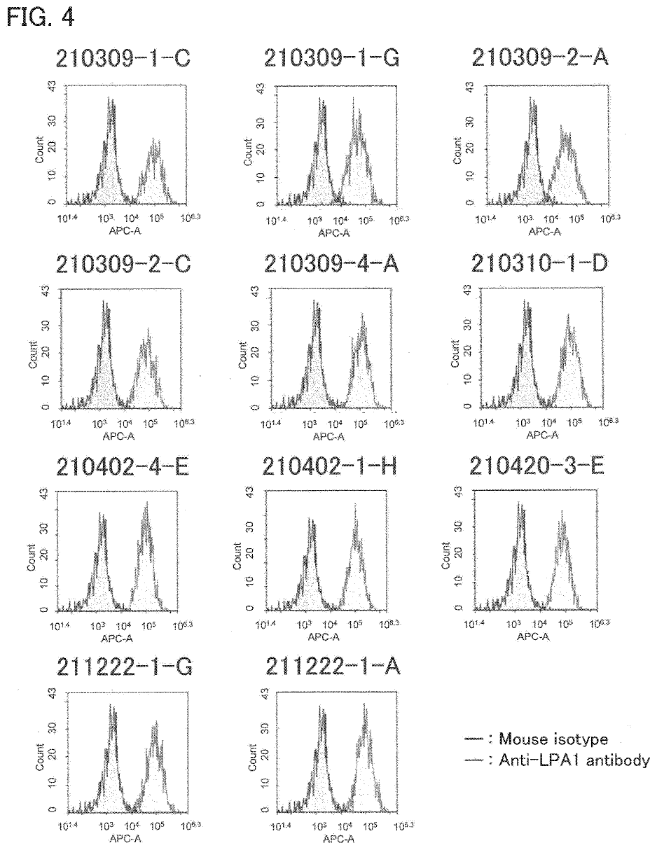

Next, the binding of the LPA1 antibody to the LPA1 endogenously expressed on the cell surface was evaluated. This was verified using a human lung-derived fibroblast cell line (IMR-90) that had been confirmed to express the human LPA1 gene. The binding properties of 10 μg/mL each of purified LPA1 antibodies and a mouse isotype control antibody were measured by a flow cytometer (Agilent Novocyte) according to the same procedure as above. As a result, the eleven antibodies were confirmed to bind specifically to IMIR-90. FIG. 4 presents histograms in which the eleven antibodies were compared with the mouse isotype control antibody.

### [Example 7] Evaluation of Intracellular cAMP Signaling Inhibition

The antibodies produced in Example 6 were subjected to the following test.

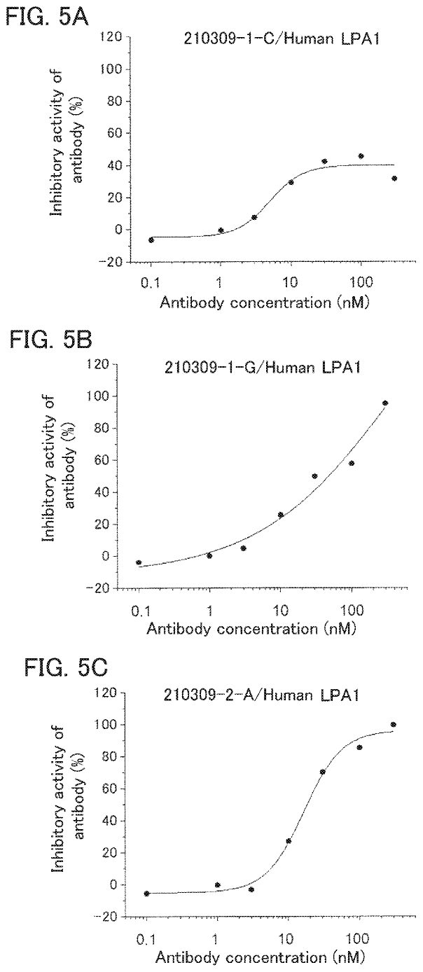

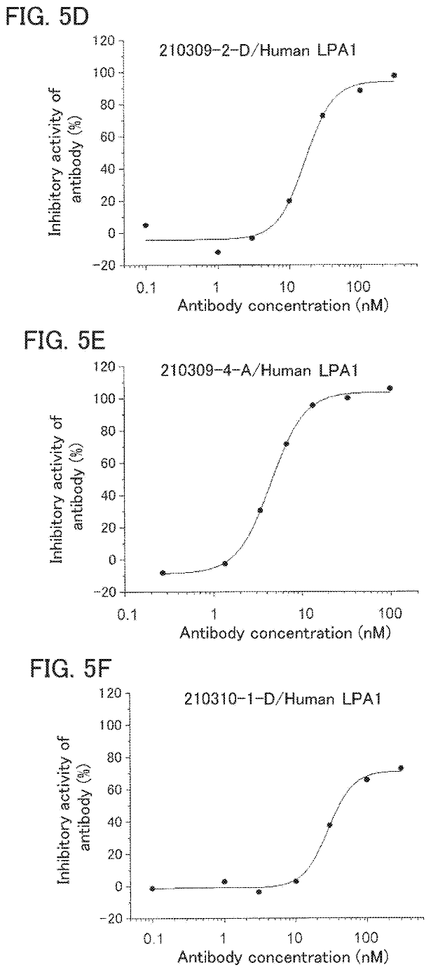

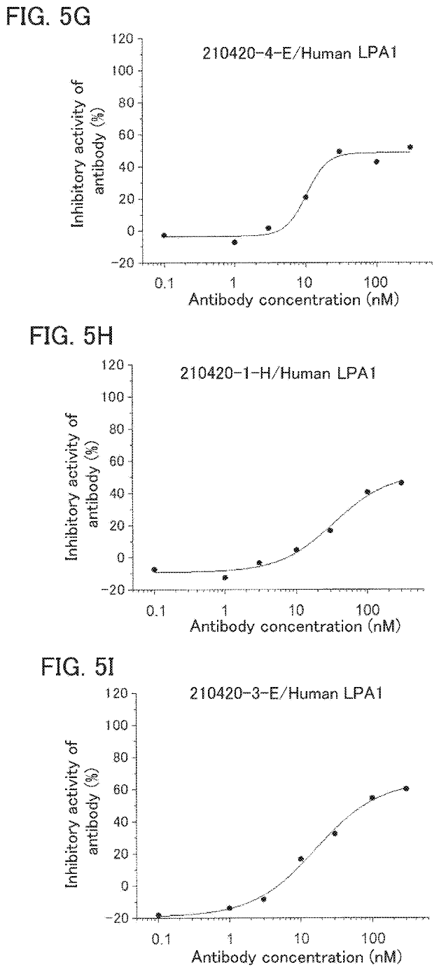

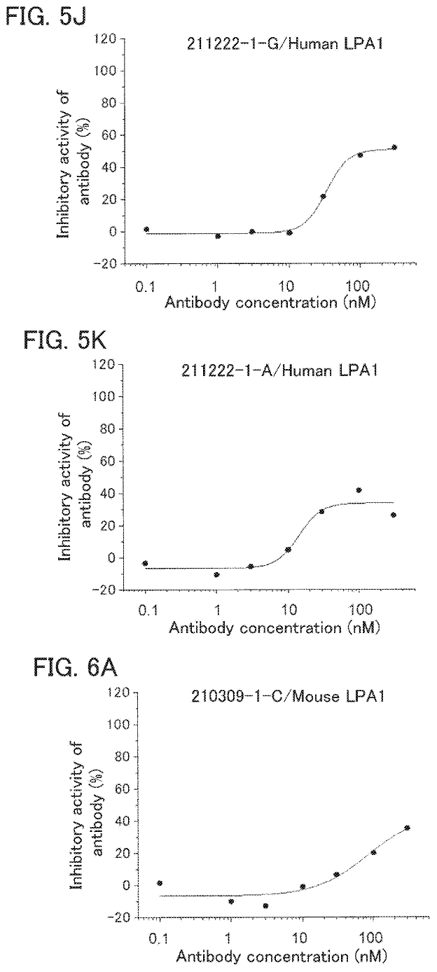

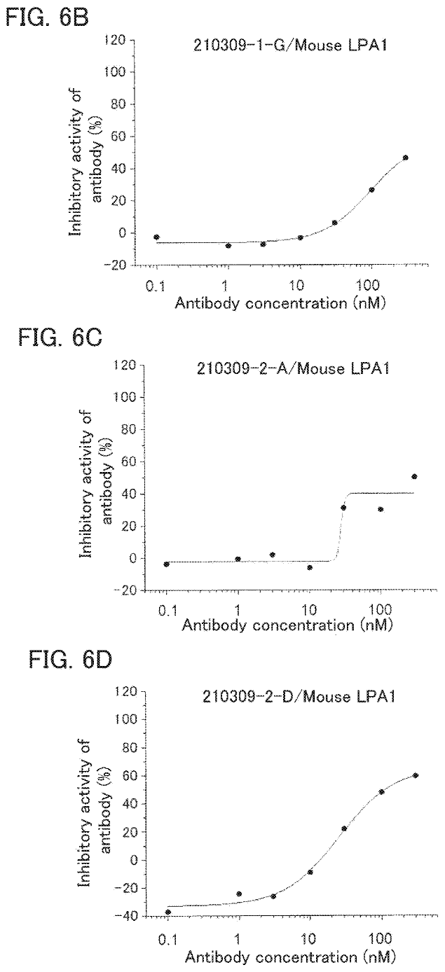

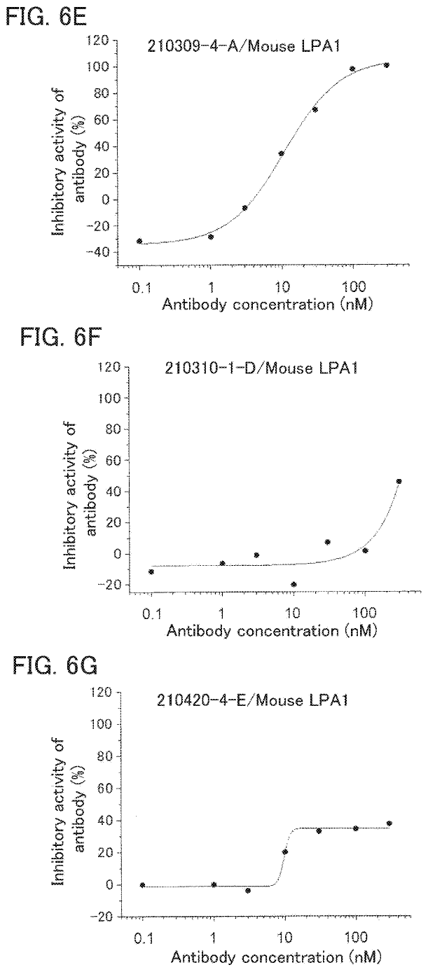

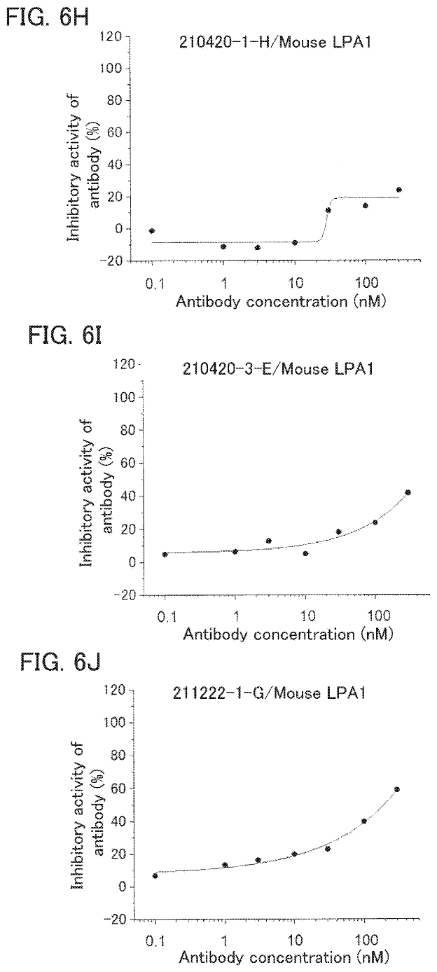

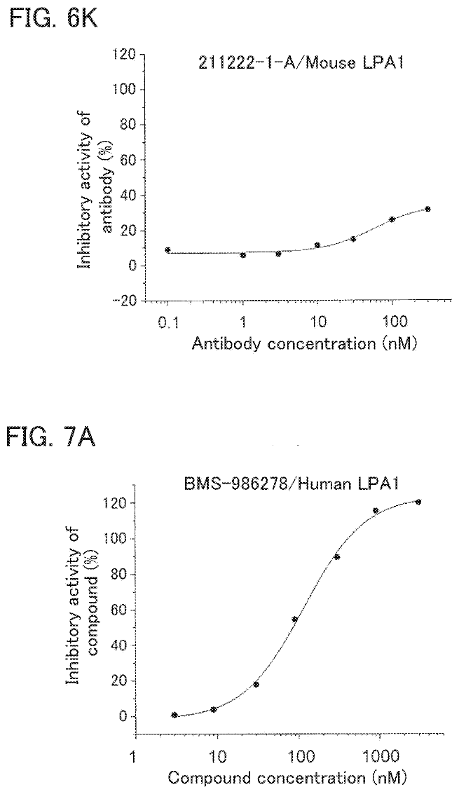

Changes in a concentration of intracellular cyclic adenosine monophosphate (cAMP) were measured using LANCE Ultra cAMP Kit (PerkinElmer, Inc.) according to the method in Example 3. To 12.5 μL of the human LPA1-stably expressing CHO cell and mouse LPA1-stably expressing CHO cell at a cell concentration of 2×10⁵~cells/mL, 6.25 μL of the purified LPA1 antibody or an LPA1 low molecule weight antagonist BMS-986278 (MedKoo Biosciences, Inc.) diluted stepwise was added and allowed to stand at room temperature for 15 minutes. Furthermore, to this mixture, 6.25 μL of a mixture of 12 μM Forskolin (Merck KGaA) (final concentration: 3 μM) and 200 nM oleoyl-L-oa-ysophosphatidic acid sodium salt (final concentration: 50 nM) was added and allowed to stand at room temperature for 30 minutes, and then the cAMP concentration was measured according to the method in Example 3. For an inhibition rate (%), a value measured in adding Forskolin and the oleoyl-L-α-lysophosphatidic acid was standardized as 0% inhibition rate and a value measured in adding only Forskolin was standardized as 100% inhibition rate, and the inhibition rate was calculated as a relative intracellular cAMP signaling inhibition rate (%). The concentration of each antibody was plotted on the vertical axis, and the inhibition rate (%) was plotted on the horizontal axis. The dose dependency of the inhibitory activity of each antibody against human LPA1 was presented in FIG. 5A to FIG. 5K, and the dose dependency of the inhibitory activity of each antibody against mouse LPA1 was presented in FIG. 6A to FIG. 6K. The results showed that all of the eleven antibodies dose-dependently inhibited lysophosphatidic acid-induced intracellular cAMP signaling in the human and mouse LPA1-expressing CHO cells, and had an activity of blocking the LPA1-dependent cell functions.

In three independent tests on the intracellular cAMP signaling inhibition, 50% inhibitory concentrations (IC50) were calculated, and their averages and standard deviations are presented in Table 5. The dose dependencies of the lysophosphatidic acid-induced intracellular cAMP signaling inhibition by BMS-986278 in the human and mouse LPA1-expressing CHO cells are presented in FIG. 7A and FIG. 7B respectively. The IC₅₀ values of BMS-986278 against the human and mouse LPA1 were 83 nM and 232 nM respectively. The results showed that all of the eleven antibodies had IC₅₀ values of the activity of blocking the human LPA1-dependent cell functions, equal to or superior to that of BMS-986278.

TABLE 5

Intracellular cAMP signaling inhibition
IC50 (nM)

| | Human LPA1-stably | Mouse LPA1-stably |
|---|---|---|
| Antibody | expressing cell | expressing cell |
| 210309-1-C | 25.1 ± 0.0 | — |
| 210309-1-G | 40.9 ± 3.2 | 197.3 ± 11.6 |
| 210309-2-A | 21.8 ± 3.5 | — |
| 210309-2-D | 18.0 ± 0.6 | 163.1 ± 62.7 |
| 210309-4-A | 5.3 ± 0.5 | 23.8 ± 6.1 |
| 210310-1-D | 60.5 ± 20.7 | — |
| 210420-4-E | 48.3 ± 34.9 | 124.0 ± 0.0 |
| 210420-1-H | 49.1 ± 0.0 | — |
| 210420-3-E | 33.1 ± 33.8 | 114.4 ± 51.2 |
| 211222-1-G | 96.8 ± 37.5 | 199.0 ± 0.0 |
| 211222-1-A | 69.9 ± 35.1 | — |

### [Example 8] Verification for Inverse Agonist Effect of LPA1 Antibody on Intracellular cAMP Signaling

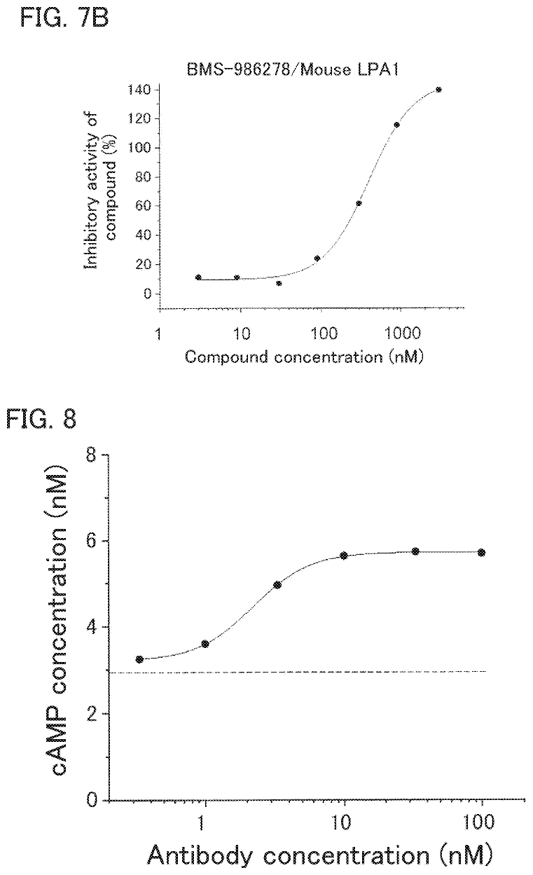

In the human LPA1-stably expressing CHO cells, the effect of 210309-4-A on changes in the intracellular cAMP concentration was evaluated in the absence of the LPA1 ligand. In other words, to 12.5 μL of human LPA1-stably expressing CHO cell at a cell concentration of 2×10⁵ cells/mL, 6.25 μL of 210309-4-A used in Example 6 and diluted stepwise was added, and allowed to stand at room temperature for 15 minutes. To this mixture, 6.25 μL of 12 μM Forskolin (final concentration: 3 μM) was further added and allowed to stand at room temperature for 30 minutes, and a cAMP concentration was measured. FIG. 8 presents a graph in which the antibody concentration is plotted on the vertical axis and the cAMP concentration (nM) is plotted on the horizontal axis. In FIG. 8, the dotted line indicates the cAMP concentration (2.9 nM) with Forskolin stimulation alone. That means, 210309-4-A showed the characteristic of the inverse agonist because it dose-dependently increased the cAMP level. As a result of subjecting the other ten LPA1 antibodies to the same test, they showed the characteristic of the inverse agonist because they dose-dependently increased the cAMP level. It was revealed that these antibodies could LPA1 ligand-independently block the LPA1 receptor-dependent cell functions.

### [Example 9] Verification for Intracellular cAMP Signaling Inhibition Manner of LPA1 Antibody

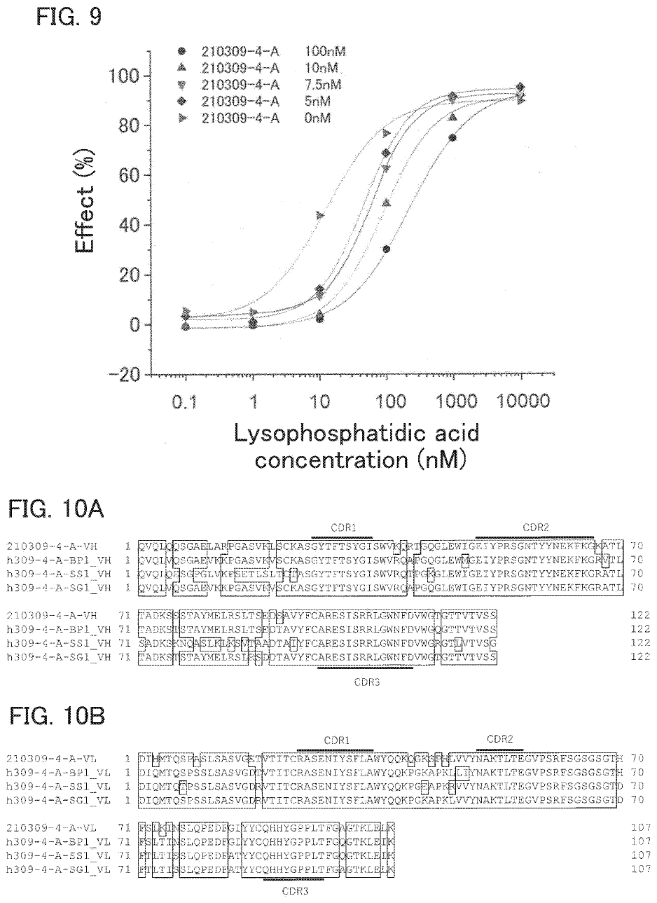

In the human LPA1-stably expressing CHO cell, the effect of various concentrations of 210309-4-A on a dose-response curve of lysophosphatidic acid as the LPA1 ligand was evaluated. In other words, 6.25 μL of 210309-4-A used in Example 6 (final concentration: 0.5, 7.5, 10, and 100 nM) at a constant concentration was added to 12.5 μL of the human LPA1-stably expressing CHO cell at a cell concentration of 2×10⁵ cells/mL, and allowed to stand at room temperature for 15 minutes. To this mixture, 6.25 μL of a mixture of 12 M Forskolin (final concentration: 3 μM) and oleoyl-L-α-lysophosphatidic acid sodium salt (final concentration: 0.1 to 1000 nM) was further added, then allowed to stand at room temperature for 30 minutes, and then a cAMP concentration was measured. FIG. 9 presents a dose-response curve of lysophosphatidic acid at each antibody concentration. The curve showed a characteristic of competitive inhibition because the dose-response curve shifted rightward as the antibody concentration increased.

### [Example 10] Preparation of Humanized Anti-LPA1 Antibody

A case of 210309-4-A will be described as an example. To prepare a humanized antibody implanted with the CDR of 210309-4-A, a human framework was selected on the basis of a homology between 210309-4-A and human germline VH and VK genes. In order to support a predicted antibody conformation of 210309-4-A, multiple antibodies with variation of the selected human germline VH and VL sequences, “h309-4-A-BP1”, “h309-4-A-SS1”, and “h309-4-A-SG1” were designed based on a computer modeling. FIG. 10A presents alignments of an h309-4-A-BP1 VH region (SEQ ID NO: 123), an h309-4-A-SS1 VH region (SEQ ID NO: 125), an h309-4-A-SG1 VH region (SEQ ID NO: 127), and a 210309-4-AVH region (SEQ ID NO: 7). FIG. 10B presents alignments of an h309-4-A-BP1 VL region (SEQ ID NO: 124), an h309-4-A-SS1 VL region (SEQ ID NO: 126), an h309-4-A-SG1 VL region (SEQ ID NO: 128), and a 210309-4-AVL region (SEQ ID NO: 9).

Artificial genes were synthesized, in which the human IgG1 heavy chain constant region (including LALA variant) (SEQ ID NO: 129) was connected to the VH region and the human IgG1 κ chain constant region (SEQ ID NO: 130) was connected to the VL region in h309-4-A-BP1, h309-4-A-SS1, and h309-4-A-SG1. As a comparable, an artificial gene of a chimeric anti-LPA1 antibody “309-4-A-Chimera” was also synthesized, in which the human IgG1 heavy chain constant region (including LALA variant) was connected to the VH region and the human IgG1 κ chain constant region was connected to the VL region in 210309-4-A in 210309-4-A. The artificial genes were introduced into CHO cells, and, from the CHO cell culture supernatant, purified antibodies were produced by affinity chromatography and subjected to the following test. As a result of assaying purities by SDS-PAGE, all the purified antibodies were confirmed to have a purity of 90% or higher.

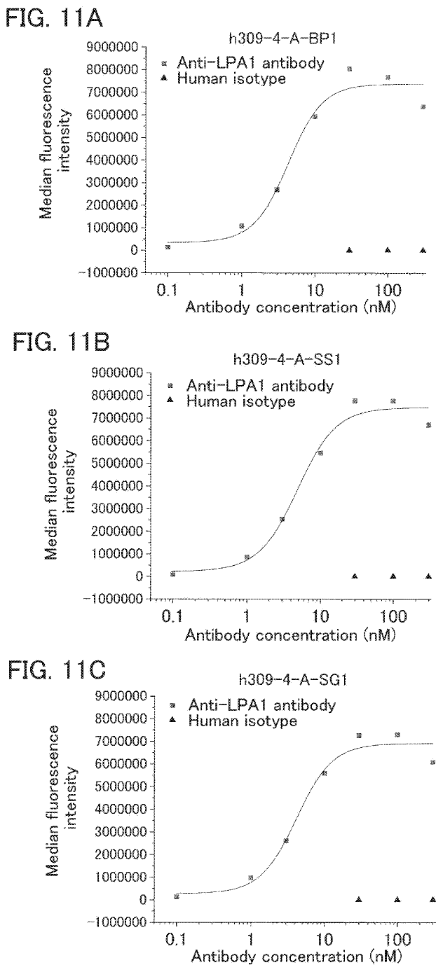

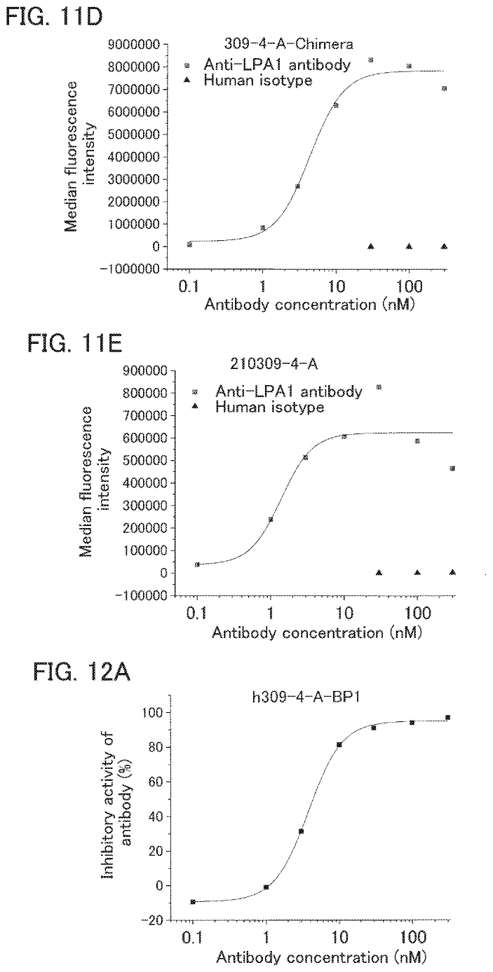

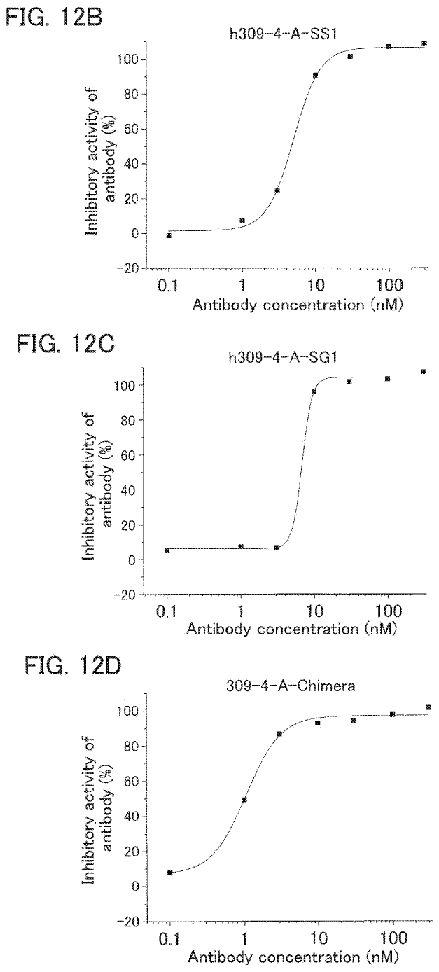

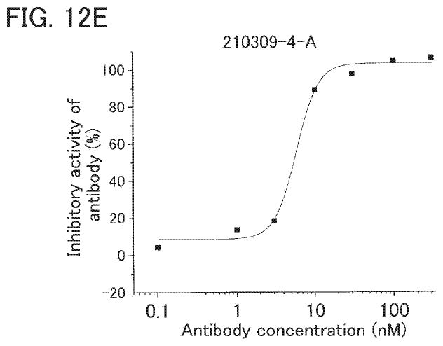

Subsequently, the binding activity of the human LPA1-stably expressing CHO cell to human LPA1 was measured according to the method in Example 6. A graph was created, in which a concentration of the antibody was plotted on the vertical axis, and a median fluorescence intensity of the flow cytometer histogram was plotted on the horizontal axis. The results of binding of each antibody to the human LPA1-stably expressing CHO cell are presented in FIG. 11A to FIG. 11E, and the calculated 50% binding concentrations (EC50) are presented in Table 6. The intracellular cAMP signaling inhibitory activity was also measured according to the method in Example 7. A graph was created, in which a concentration and an inhibition rate (%) of each antibody were plotted on the vertical axis and the horizontal axis respectively. The dose dependencies of the inhibitory activity of each antibody against human LPA1 are presented in FIG. 12A to FIG. 12E, and the calculated 50% inhibitory concentrations (ICSO) of each antibody are presented in Table 7.

As shown in these results, the binding and inhibitory activities of 309-4-A-Chimera were equivalent to those of 210309-4-A, indicating that the conversion of the human antibody to the constant region did not affect the binding and inhibitory activities. Furthermore, the binding and inhibitory activities of h309-4-A-BP1, h309-4-A-SS1, and h309-4-A-SG1 designed as humanized LPA1 antibodies were equivalent to those of 309-4-A-Chimera, indicating that the binding and inhibitory activities of all designs were maintained after the humanization.

TABLE 6

| | Binding to LPA1-stably expressing cell |
|---|---|
| | EC50 (nM) |
| Antibody | Human LPA1-stably expressing cell |
| h309-4-A-BP1 | 4.3 |
| h309-4-A-SS1 | 4.9 |
| h309-4-A-SG1 | 4.2 |
| 309-4-A-Chimera | 4.4 |
| 210309-4-A | 1.4 |

TABLE 7

| | Intracellular cAMP signaling inhibition |
|---|---|
| | IC50 (nM) |
| Antibody | Human LPA1-stably expressing cell |
| h309-4-A-BP1 | 4.4 |
| h309-4-A-SS1 | 5.2 |
| h309-4-A-SG1 | 5.7 |
| 309-4-A-Chimera | 1.0 |
| 210309-4-A | 4.5 |

The amino acid sequences of SEQ ID NO: 123 to 130 are presented in Table 8.

TABLE 8

| SEQ ID | | | |
|---|---|---|---|
| NO: | Antibody ID | Content | Amino acid sequence |
| 123 | h309-4-A-BP1 | Heavy chain | QVQLVQSGAEVKKPGASVKVSCKASGYTFTSYGISWVRQAPGQGL |
| | | variable region | EWMGEIYPRSGNTYYNEKFKGRVTLTADKSTSTAYMELRSLTSED |
| | | | TAVYFCARESISRRLGWNEDVWGQGTTVTVSS |
| 124 | | Light chain | DIQMTQSPSSLSASVGDTVTITCRASENIYSFLAWYQQKPGKAPK |
| | | variable region | LLIYNAKTLTEGVPSRFSGSGSGTHFSLTINSLQPEDFGIYYCQH |
| | | | HYGPPLTFGQGTKLEIK |
| 125 | h309-4-A-SS1 | Heavy chain | QVQLQESGPGLVKPSETLSLTCTASGYTFTSYGISWVRQTPGKGL |
| | | variable region | EWIGEIYPRSGNTYYNEKFKGRATLSADKSKNQASLKLKSVTAAD |
| | | | TAIYFCARESISRRLGWNFDVWGRGTLVTVSG |
| 126 | | Light chain | DIQMTQTPSSLSASVGDRVTITCRASENIYSFLAWYQQKPGEAPK |
| | | variable region | RVVYNAKTLTEGVPSRFSGSGSGTDFTLTISSLQPEDFATYYCQH |
| | | | HYGPPLTFGQGTKLEIK |
| 127 | h309-4-A-SG1 | Heavy chain | QVQLVQSGAEVKKPGASVKVSCKASGYTFTSYGISWVRQAPGQGL |
| | | variable region | EWIGEIYPRSGNTYYNEKFKGRATLTADKSTSTAYMELRSLRSDD |
| | | | TAVYFCARESISRRLGWNEDVWGTGTTVTVSS |
| 128 | | Light chain | DIQMTQSPSSLSASVGDRVTITCRASENIYSFLAWYQQKPGKAPK |
| | | variable region | LVVYNAKTLTEGVPSRFSGSGSGTDFTLTISSLQPEDFATYYCQH |
| | | | HYGPPLTFGAGTKLELK |
| 129 | HC-LALA | Heavy chain | ASTKGPSVFPLAPSSKSTSGGTAALGCLVKDYFPEPVTVSWNSGA |
| | | constant region | LTSGVHTFPAVLQSSGLYSLSSVVTVPSSSLGTQTYICNVNHKPS |
| | | (including LALA | NTKVDKKVEPKSCDKTHTCPPCPAPEAAGGPSVFLFPPKPKDTLM |
| | | variant) | ISRTPEVTCVVVDVSHEDPEVKENWYVDGVEVHNAKTKPREEQYN |
| | | | STYRVVSVLTVLHQDWLNGKEYKCKVSNKALPAPIEKTISKAKGQ |
| | | | PREPQVYTLPPSRDELTKNQVSLTCLVKGFYPSDIAVEWESNGQP |
| | | | ENNYKTTPPVLDSDGSFFLYSKLTVDKSRWQQGNVFSCSVMHEAL |
| | | | HNHYTQKSLSLSPGK |
| 130 | LC | Light chain | RTVAAPSVFIFPPSDEQLKSGTASVVCLLNNFYPREAKVQWKVDN |
| | | constant region | ALQSGNSQESVTEQDSKDSTYSLSSTLTLSKADYEKHKVYACEVT |
| | | | HQGLSSPVTKSENRGEC |

1. An antibody that specifically binds to an extracellular domain of human LPA1, wherein
the antibody does not specifically bind to an extracellular domain of human LPA2,
the antibody does not specifically bind to an extracellular domain of human LPA3,
the antibody comprises an activity of blocking an LPA1-dependent cell function,
the antibody comprises an inverse agonist effect, and
the antibody satisfies any one of the following (A1) to (A11):
(A1) comprising a heavy chain CDR1 including an amino acid sequence represented by SEQ ID NO: 1, a heavy chain CDR2 including an amino acid sequence represented by SEQ ID NO: 2, and a heavy chain CDR3 including an amino acid sequence represented by SEQ ID NO: 3;
(A2) comprising a heavy chain CDR1 including an amino acid sequence represented by SEQ ID NO: 11, a heavy chain CDR2 including an amino acid sequence represented by SEQ ID NO: 12, and a heavy chain CDR3 including an amino acid sequence represented by SEQ ID NO: 13;
(A3) comprising a heavy chain CDR1 including an amino acid sequence represented by SEQ ID NO: 21, a heavy chain CDR2 including an amino acid sequence represented by SEQ ID NO: 22, and a heavy chain CDR3 including an amino acid sequence represented by SEQ ID NO: 23;
(A4) comprising a heavy chain CDR1 including an amino acid sequence represented by SEQ ID NO: 31, a heavy chain CDR2 including an amino acid sequence represented by SEQ ID NO: 32, and a heavy chain CDR3 including an amino acid sequence represented by SEQ ID NO: 33;
(A5) comprising a heavy chain CDR1 including an amino acid sequence represented by SEQ ID NO: 41, a heavy chain CDR2 including an amino acid sequence represented by SEQ ID NO: 42, and a heavy chain CDR3 including an amino acid sequence represented by SEQ ID NO: 43;
(A6) comprising a heavy chain CDR1 including an amino acid sequence represented by SEQ ID NO: 51, a heavy chain CDR2 including an amino acid sequence represented by SEQ ID NO: 52, and a heavy chain CDR3 including an amino acid sequence represented by SEQ ID NO: 53;
(A7) comprising a heavy chain CDR1 including an amino acid sequence represented by SEQ ID NO: 61, a heavy chain CDR2 including an amino acid sequence represented by SEQ ID NO: 62, and a heavy chain CDR3 including an amino acid sequence represented by SEQ ID NO: 63;
(A8) comprising a heavy chain CDR1 including an amino acid sequence represented by SEQ ID NO: 71, a heavy chain CDR2 including an amino acid sequence represented by SEQ ID NO: 72, and a heavy chain CDR3 including an amino acid sequence represented by SEQ ID NO: 73;
(A9) comprising a heavy chain CDR1 including an amino acid sequence represented by SEQ ID NO: 81, a heavy chain CDR2 including an amino acid sequence represented by SEQ ID NO: 82, and a heavy chain CDR3 including an amino acid sequence represented by SEQ ID NO: 83;
(A10) comprising a heavy chain CDR1 including an amino acid sequence represented by SEQ ID NO: 91, a heavy chain CDR2 including an amino acid sequence represented by SEQ ID NO: 92, and a heavy chain CDR3 including an amino acid sequence represented by SEQ ID NO: 93; and
(A11) comprising a heavy chain CDR1 including an amino acid sequence represented by SEQ ID NO: 101, a heavy chain CDR2 including an amino acid sequence represented by SEQ ID NO: 102, and a heavy chain CDR3 including an amino acid sequence represented by SEQ ID NO: 103.

2. The antibody according to claim 1, wherein the antibody is a humanized antibody or a chimeric antibody.

3. The antibody according to claim 1, wherein the antibody is a multispecific antibody.

4. The antibody according to claim 1, wherein the antibody is a modified antibody bonded with another molecule.

5. The antibody according to claim 4, wherein the modified antibody is an antibody-drug conjugate.

6. A nucleic acid encoding the antibody according to claim 1.

7. A cell comprising the nucleic acid according to claim 6.

8. A medicament comprising the antibody according to claim 1 as an active ingredient.

9. The medicament according to claim 8, wherein the medicament is used for treating tissue fibrosis, cell proliferative disease, pain, inflammatory disease, autoimmune disease, metabolic disease, cardiovascular disorder, urological disease, or ophthalmological disease.

10. The medicament according to claim 9, wherein
the medicament is used for treating tissue fibrosis, and
the tissue fibrosis is hepatic fibrosis, kidney fibrosis, pulmonary fibrosis, skin fibrosis, cardiovascular fibrosis, or gastrointestinal fibrosis.

11. The antibody according to claim 2, wherein the antibody is a multispecific antibody.

12. The antibody according to claim 2, wherein the antibody is a modified antibody bonded with another molecule.

13. The antibody according to claim 12, wherein the modified antibody is an antibody-drug conjugate.

14. A nucleic acid encoding the antibody according to claim 2.

15. A cell comprising the nucleic acid according to claim 14.

16. A medicament comprising the antibody according to claim 2 as an active ingredient.

17. The medicament according to claim 16, wherein the medicament is used for treating tissue fibrosis, cell proliferative disease, pain, inflammatory disease, autoimmune disease, metabolic disease, cardiovascular disorder, urological disease, or ophthalmological disease.

18. The medicament according to claim 17, wherein
the medicament is used for treating tissue fibrosis, and
the tissue fibrosis is hepatic fibrosis, kidney fibrosis, pulmonary fibrosis, skin fibrosis, cardiovascular fibrosis, or gastrointestinal fibrosis.


SEQUENCE LISTING (130 sequences)

SEQ ID NO: 1 - protein, 10 aa, Mus musculus
GYTFTSYGIS

SEQ ID NO: 2 - protein, 17 aa, Mus musculus
EIYPRSGNTYYNEKFKG

SEQ ID NO: 3 - protein, 15 aa, Mus musculus
ARESISRRLGWNFDV

SEQ ID NO: 4 - protein, 11 aa, Mus musculus
RASENIYSFLA

SEQ ID NO: 5 - protein, 7 aa, Mus musculus
NAKTLTE

SEQ ID NO: 6 - protein, 9 aa, Mus musculus
QHHYGPPLT

SEQ ID NO: 7 - protein, 122 aa, Mus musculus
QVQLQQSGAELARPGASVKLSCKASGYTFTSYGISWVKQRTGQGLEWIGEIYPRSGNTYYNEKFKGKATLTADKSSSTAYMELRSLTSEDSAVYFCARESISRRLGWNFDVWGTGTTVTVSS

SEQ ID NO: 8 - DNA, 366 bp, Mus musculus
  source 1..366: mol_type = other DNA
  CDS 1..366: protein_id = 7; translation = QVQLQQSGAELARPGASVKLSCKASGYTFTSYGISWVKQRTGQG LEWIGEIYPRSGNTYYNEKFKGKATLTADKSSSTAYMELRSLTSEDSAVYFCARESIS RRLGWNFDVWGTGTTVTVSS
caggttcagctgcagcagtctggagctgagctggcgaggcctggggcttcagtgaagctgtcctgcaaggcttctggctacaccttcacaagctatggtataagctgggtgaagcagagaactggacagggccttgagtggattggagagatttatcctagaagtggtaatacttactacaatgagaagttcaagggcaaggccacactgactgcagacaaatcctccagcacagcgtacatggagctccgcagcctgacatctgaggactctgcggtctatttctgtgcaagagagtctatatcacgacggctaggctggaacttcgatgtctggggcacagggaccacggtcaccgtctcctca

SEQ ID NO: 9 - protein, 107 aa, Mus musculus
DIHMTQSPASLSASVGETVTITCRASENIYSFLAWYQQKQGKSPHLVVYNAKTLTEGVPSRFSGSGSGTHFSLKINSLQPEDFGIYYCQHHYGPPLTFGAGTKLELK

SEQ ID NO: 10 - DNA, 321 bp, Mus musculus
  source 1..321: mol_type = other DNA
  CDS 1..321: protein_id = 9; translation = DIHMTQSPASLSASVGETVTITCRASENIYSFLAWYQQKQGKSP HLVVYNAKTLTEGVPSRFSGSGSGTHFSLKINSLQPEDFGIYYCQHHYGPPLTFGAGT KLELK
gacatccacatgactcagtctccagcctccctatctgcatctgtgggagaaactgtcaccatcacatgtcgagcaagtgagaatatttacagttttttagcatggtatcagcagaaacagggaaaatctcctcacctcgtggtctacaatgcaaagaccttaacagaaggtgtgccatcaaggttcagtggcagtggatcaggcacacacttttctctgaagatcaacagccttcagcctgaagattttgggatttattactgtcaacatcattatggtcctcctctcacgttcggtgctgggaccaagctggagctgaaa

SEQ ID NO: 11 - protein, 10 aa, Mus musculus
GYSFTAYGIS

SEQ ID NO: 12 - protein, 17 aa, Mus musculus
EIYPRSGNTYYSEKFKG

SEQ ID NO: 13 - protein, 15 aa, Mus musculus
ARESLLKRLGWYFDV

SEQ ID NO: 14 - protein, 11 aa, Mus musculus
RASQNIYSFLA

SEQ ID NO: 15 - protein, 7 aa, Mus musculus
NAKTLAE

SEQ ID NO: 16 - protein, 9 aa, Mus musculus
QHHYGPPLT

SEQ ID NO: 17 - protein, 122 aa, Mus musculus
QVHLQQSGAELARPGASVKLSCKASGYSFTAYGISWVQQRTGQGLEWIGEIYPRSGNTYYSEKFKGKATLTADKSSSTAYMELRSLTSEDSAVYFCARESLLKRLGWYFDVWGTGTTVTVSS

SEQ ID NO: 18 - DNA, 366 bp, Mus musculus
  source 1..366: mol_type = other DNA
  CDS 1..366: protein_id = 17; translation = QVHLQQSGAELARPGASVKLSCKASGYSFTAYGISWVQQRTGQG LEWIGEIYPRSGNTYYSEKFKGKATLTADKSSSTAYMELRSLTSEDSAVYFCARESLL KRLGWYFDVWGTGTTVTVSS
caggttcatctgcagcagtctggagctgagctggcgaggcctggggcttcagtgaagctgtcctgcaaggcttctggctacagcttcacagcctatggtataagctgggtgcagcagagaactggacagggccttgagtggattggagagatttatccaagaagtggtaatacttactacagtgagaagttcaagggcaaggccacactgactgcagacaaatcctccagtacagcgtacatggagctccgcagcctgacatctgaggactctgcggtctatttctgtgcaagagagtctctattaaaacggctaggctggtacttcgatgtctggggcacagggaccacggtcaccgtctcctca

SEQ ID NO: 19 - protein, 107 aa, Mus musculus
DIQMTQSPASLSASVGETVTITCRASQNIYSFLAWYQQKQGNSPQLLVYNAKTLAEGVPSRFSGSGSGTQFSLKINSLQPEDFGSYYCQHHYGPPLTFGVGTKLELK

SEQ ID NO: 20 - DNA, 321 bp, Mus musculus
  source 1..321: mol_type = other DNA
  CDS 1..321: protein_id = 19; translation = DIQMTQSPASLSASVGETVTITCRASQNIYSFLAWYQQKQGNSP QLLVYNAKTLAEGVPSRFSGSGSGTQFSLKINSLQPEDFGSYYCQHHYGPPLTFGVGT KLELK
gacatccagatgactcagtctccagcctccctatctgcatctgtgggagaaactgtcaccatcacatgtcgagcaagtcagaatatttacagttttttagcatggtatcagcagaaacagggaaattctcctcagctcctggtctataatgcaaaaaccttagcagaaggtgtgccatcaaggttcagtggcagtggatcaggcacacagttttctctgaagatcaacagcctgcagcctgaagattttgggagttattactgtcaacatcattatggtcctcctctcacgttcggtgttgggaccaagctggagctgaaa

SEQ ID NO: 21 - protein, 10 aa, Mus musculus
GYTFTSYGIS

SEQ ID NO: 22 - protein, 17 aa, Mus musculus
EIYPRSGVTYYNEKFKG

SEQ ID NO: 23 - protein, 15 aa, Mus musculus
ARESISLRLGWYFDV

SEQ ID NO: 24 - protein, 11 aa, Mus musculus
RASENIYRFLA

SEQ ID NO: 25 - protein, 7 aa, Mus musculus
NAKTLAE

SEQ ID NO: 26 - protein, 9 aa, Mus musculus
QHHYGPPLT

SEQ ID NO: 27 - protein, 122 aa, Mus musculus
QVQLQQSGAELARPGASMKLSCRASGYTFTSYGISWVKQRTGQGLEWIGEIYPRSGVTYYNEKFKGRATLTADKSSSTAFMELRSLTSEDSAVYFCARESISLRLGWYFDVWGTGTTVTVSS

SEQ ID NO: 28 - DNA, 366 bp, Mus musculus
  source 1..366: mol_type = other DNA
  CDS 1..366: protein_id = 27; translation = QVQLQQSGAELARPGASMKLSCRASGYTFTSYGISWVKQRTGQG LEWIGEIYPRSGVTYYNEKFKGRATLTADKSSSTAFMELRSLTSEDSAVYFCARESIS LRLGWYFDVWGTGTTVTVSS
caggttcagctgcagcagtctggagctgagctggcgaggcctggggcttcaatgaagctgtcctgcagggcttctggctacaccttcacaagctatggtataagctgggtgaagcagagaactggacagggccttgagtggattggagagatttatcccagaagtggtgttacttactacaatgagaagttcaagggtagggccacactgactgcagacaaatcctccagcacagcgttcatggagctccgcagcctgacatctgaggactctgcggtctatttctgtgcaagagagtctatatcactacgactaggctggtacttcgatgtctggggcacagggaccacggtcaccgtctcctca

SEQ ID NO: 29 - protein, 107 aa, Mus musculus
DIQMTQSPASLSASVGDTVTITCRASENIYRFLAWYQQKQRKSPHLLVYNAKTLAEGVPSRFSGSGSGTQFSLKINSLQPEDFGSYYCQHHYGPPLTFGAGTKLELT

SEQ ID NO: 30 - DNA, 321 bp, Mus musculus
  source 1..321: mol_type = other DNA
  CDS 1..321: protein_id = 29; translation = DIQMTQSPASLSASVGDTVTITCRASENIYRFLAWYQQKQRKSP HLLVYNAKTLAEGVPSRFSGSGSGTQFSLKINSLQPEDFGSYYCQHHYGPPLTFGAGT KLELT
gacatccagatgactcagtctccagcctccctatctgcatctgtgggagatactgtcaccatcacatgtcgagcaagtgagaatatttacaggtttttagcatggtatcagcagaaacagagaaaatctccgcacctcctggtctataatgcaaaaaccttagcagaaggtgtgccatcaaggttcagtggcagtggatcaggcacacagttttctctgaagatcaacagtctgcagcctgaagattttgggagttattattgtcaacatcattatggtcctccactcacgttcggtgctgggaccaagctggagctgaca

SEQ ID NO: 31 - protein, 10 aa, Mus musculus
GYTFTSYGIS

SEQ ID NO: 32 - protein, 17 aa, Mus musculus
EIYPRSGNTYYNEKFKG

SEQ ID NO: 33 - protein, 15 aa, Mus musculus
ARESILLRLGWYFDV

SEQ ID NO: 34 - protein, 11 aa, Mus musculus
RASENIYRFLA

SEQ ID NO: 35 - protein, 7 aa, Mus musculus
NAKTLAE

SEQ ID NO: 36 - protein, 9 aa, Mus musculus
QHHYGPPLT

SEQ ID NO: 37 - protein, 122 aa, Mus musculus
QVQLQQSGAELARPGASVKLSCKASGYTFTSYGISWVKQRTGQGLEWIGEIYPRSGNTYYNEKFKGRATLTADKSSSTAYMELRSLTSEDSAVYFCARESILLRLGWYFDVWGTGTTVTVSS

SEQ ID NO: 38 - DNA, 366 bp, Mus musculus
  source 1..366: mol_type = other DNA
  CDS 1..366: protein_id = 37; translation = QVQLQQSGAELARPGASVKLSCKASGYTFTSYGISWVKQRTGQG LEWIGEIYPRSGNTYYNEKFKGRATLTADKSSSTAYMELRSLTSEDSAVYFCARESIL LRLGWYFDVWGTGTTVTVSS
caggttcagctgcagcagtctggagctgagctggcgaggcctggggcttcagtgaagctgtcctgcaaggcttctggctacaccttcacaagctatggtataagctgggtgaagcagagaactggacagggccttgagtggattggagagatttatcctagaagtggtaatacttactacaatgagaagttcaagggcagggccacactgactgcagacaaatcctccagcacagcgtacatggagctccgcagcctgacatctgaggactctgcggtctatttctgtgcaagagagtctatattactacggctaggctggtacttcgatgtctggggcacagggaccacggtcaccgtctcctca

SEQ ID NO: 39 - protein, 107 aa, Mus musculus
DIQMTQSPASLSASVGGTVTITCRASENIYRFLAWYQQKQGKSPQLLVYNAKTLAEGVPSRFSGSGSGTQFSLKINSLQPEDFGIYYCQHHYGPPLTFGAGTKLELK

SEQ ID NO: 40 - DNA, 321 bp, Mus musculus
  source 1..321: mol_type = other DNA
  CDS 1..321: protein_id = 39; translation = DIQMTQSPASLSASVGGTVTITCRASENIYRFLAWYQQKQGKSP QLLVYNAKTLAEGVPSRFSGSGSGTQFSLKINSLQPEDFGIYYCQHHYGPPLTFGAGT KLELK
gacatccagatgactcagtctccagcctccctatctgcatctgtgggaggaactgtcaccatcacatgtcgagcaagtgagaatatttacaggtttttagcatggtatcagcagaaacagggaaaatctcctcagctcctggtctataatgcaaaaaccttagcagaaggtgtgccatcaaggttcagtggcagtggatcaggcacacagttttctctgaagatcaacagccttcagcctgaagattttgggatttattactgtcaacatcattatggtcctcctctcacgttcggtgctgggaccaagctggagctgaaa

SEQ ID NO: 41 - protein, 10 aa, Mus musculus
GYTFTSYGIS

SEQ ID NO: 42 - protein, 17 aa, Mus musculus
EIYPRSGNTYYNEKVKG

SEQ ID NO: 43 - protein, 15 aa, Mus musculus
ARESIFLRLGWYFDV

SEQ ID NO: 44 - protein, 11 aa, Mus musculus
RASDNIYRFLA

SEQ ID NO: 45 - protein, 7 aa, Mus musculus
NAKTLAE

SEQ ID NO: 46 - protein, 9 aa, Mus musculus
QHHYGPPLT

SEQ ID NO: 47 - protein, 122 aa, Mus musculus
QVQLQQSGAELARPGASVKLSCKASGYTFTSYGISWVKQRTGQGLEWIGEIYPRSGNTYYNEKVKGKATLTADKSSSTAYMELRSLTSEDSAVYFCARESIFLRLGWYFDVWGTGTTVTVSS

SEQ ID NO: 48 - DNA, 366 bp, Mus musculus
  source 1..366: mol_type = other DNA
  CDS 1..366: protein_id = 47; translation = QVQLQQSGAELARPGASVKLSCKASGYTFTSYGISWVKQRTGQG LEWIGEIYPRSGNTYYNEKVKGKATLTADKSSSTAYMELRSLTSEDSAVYFCARESIF LRLGWYFDVWGTGTTVTVSS
caggttcagctgcagcagtctggagctgagctggcgaggcctggggcttcagtgaagctgtcctgcaaggcttctggctacaccttcacaagctatggtataagttgggtgaagcagagaactggacagggccttgagtggattggagagatttatcctagaagtggtaatacttactacaatgagaaggtcaagggcaaggccacactgactgcagacaaatcctccagcacagcgtacatggagctccgcagcctgacatctgaggactctgcggtctatttctgtgcaagagagtctatattcctacgtctaggctggtacttcgatgtctggggcacagggaccacggtcaccgtctcctca

SEQ ID NO: 49 - protein, 107 aa, Mus musculus
DIQMTQSPASLSASVGETVTITCRASDNIYRFLAWYQQKQGKSPQLLVYNAKTLAEGVPSRFSGSGTGTQFSLKINSLQPEDFGIYYCQHHYGPPLTFGAGTKLELK

SEQ ID NO: 50 - DNA, 321 bp, Mus musculus
  source 1..321: mol_type = other DNA
  CDS 1..321: protein_id = 49; translation = DIQMTQSPASLSASVGETVTITCRASDNIYRFLAWYQQKQGKSP QLLVYNAKTLAEGVPSRFSGSGTGTQFSLKINSLQPEDFGIYYCQHHYGPPLTFGAGT KLELK
gacatccagatgactcagtctccagcctccctatctgcatctgtgggagaaactgtcaccatcacatgtcgagcaagtgacaatatttacaggtttttagcatggtatcagcagaaacagggaaaatctcctcagctcctggtctataatgcaaaaaccttagcagaaggtgtgccatcaaggttcagtggcagtggaacaggcacacagttttctctgaagatcaacagtcttcagcctgaagattttgggatttattactgtcaacatcattatggtcctcctctcacgttcggtgctgggaccaagctggagctgaaa

SEQ ID NO: 51 - protein, 10 aa, Mus musculus
GYTFTSFGVN

SEQ ID NO: 52 - protein, 17 aa, Mus musculus
QIYPRTGTTYHNERFKG

SEQ ID NO: 53 - protein, 11 aa, Mus musculus
AREGDRYSLAY

SEQ ID NO: 54 - protein, 11 aa, Mus musculus
RASENIYRFLA

SEQ ID NO: 55 - protein, 7 aa, Mus musculus
NAKTLVE

SEQ ID NO: 56 - protein, 9 aa, Mus musculus
QHHYGIPLT

SEQ ID NO: 57 - protein, 118 aa, Mus musculus
QVQVQQSGAELARPGASVRLSCKASGYTFTSFGVNWVKQRTGQGLEWIGQIYPRTGTTYHNERFKGKATLTTDKSSSTAYMELRSLTSEDSAVYFCAREGDRYSLAYWGQGTLVTVSA

SEQ ID NO: 58 - DNA, 354 bp, Mus musculus
  source 1..354: mol_type = other DNA
  CDS 1..354: protein_id = 57; translation = QVQVQQSGAELARPGASVRLSCKASGYTFTSFGVNWVKQRTGQG LEWIGQIYPRTGTTYHNERFKGKATLTTDKSSSTAYMELRSLTSEDSAVYFCAREGDR YSLAYWGQGTLVTVSA
caggttcaggtacagcagtccggagctgagctggcgaggcctggggcatcagtgaggctgtcctgcaaggcttctggctacaccttcacaagctttggtgtaaactgggtgaagcagagaactggacagggccttgagtggattggacagatttatcctagaactggtactacttaccacaatgagaggttcaagggcaaggccacactgactacagacaaatcctccagcacagcgtacatggagctccgcagcctgacatctgaggactctgcggtctatttctgtgcacgagagggagataggtactcacttgcttactggggccaagggactctggtcactgtctctgca

SEQ ID NO: 59 - protein, 107 aa, Mus musculus
DIQMTQSPASLSASVGETVTITCRASENIYRFLAWYQQKQGKSPQLLVYNAKTLVEGVPSRFSGSGSGTQFSLKINNLQPEDFGSYYCQHHYGIPLTFGAGTKLELK

SEQ ID NO: 60 - DNA, 321 bp, Mus musculus
  source 1..321: mol_type = other DNA
  CDS 1..321: protein_id = 59; translation = DIQMTQSPASLSASVGETVTITCRASENIYRFLAWYQQKQGKSP QLLVYNAKTLVEGVPSRFSGSGSGTQFSLKINNLQPEDFGSYYCQHHYGIPLTFGAGT KLELK
gacatccagatgactcagtctccagcctccctatctgcatctgtgggagaaactgtcaccatcacatgtcgagcaagtgagaatatttacaggtttttagcatggtatcagcagaaacagggaaaatctcctcagctcctggtctataatgcaaaaaccttagtagaaggtgtgccatcaaggttcagtggcagtggatcaggcacacagttttctctgaagatcaacaacctacagcctgaagattttgggagttattactgtcaacatcattatggtattccgctcacgttcggtgctgggaccaagctggagctgaaa

SEQ ID NO: 61 - protein, 10 aa, Mus musculus
GYTFASFGIS

SEQ ID NO: 62 - protein, 17 aa, Mus musculus
EIYPRSGTTYFNEKFRG

SEQ ID NO: 63 - protein, 12 aa, Mus musculus
AREGVRRYAMDY

SEQ ID NO: 64 - protein, 11 aa, Mus musculus
RASGNIYSFLA

SEQ ID NO: 65 - protein, 7 aa, Mus musculus
NAKTLAE

SEQ ID NO: 66 - protein, 9 aa, Mus musculus
QHHYGIPLT

SEQ ID NO: 67 - protein, 119 aa, Mus musculus
QLHLQQSGAELARPGASVRLSCKASGYTFASFGISWVKQRTGQGLEYIGEIYPRSGTTYFNEKFRGKATLSADKSSSTAYMELRSLTSEDSAVYFCAREGVRRYAMDYWGQGTSVTVSS

SEQ ID NO: 68 - DNA, 357 bp, Mus musculus
  source 1..357: mol_type = other DNA
  CDS 1..357: protein_id = 67; translation = QLHLQQSGAELARPGASVRLSCKASGYTFASFGISWVKQRTGQG LEYIGEIYPRSGTTYFNEKFRGKATLSADKSSSTAYMELRSLTSEDSAVYFCAREGVR RYAMDYWGQGTSVTVSS
cagcttcacctgcagcagtctggagctgagctggcgaggcctggggcttcagtgaggctgtcctgcaaggcttctggctacaccttcgcaagttttggtataagctgggtgaagcagagaactggacagggccttgaatacattggagagatttatcctagaagtggtactacttacttcaatgagaagttcaggggcaaggccacactgagtgcagacaaatcatccagcacagcgtacatggagctccgcagcctgacatctgaggactctgcggtctatttctgtgcaagagagggagtacgacggtatgctatggactactggggtcaaggaacctcagtcaccgtctcctca

SEQ ID NO: 69 - protein, 107 aa, Mus musculus
DIQVTQSPASLSASVGETVTITCRASGNIYSFLAWYQQKQGKSPQLLVYNAKTLAEGVPSRFSGSGSGTQFSLKISSLQPEDFGSYYCQHHYGIPLTFGAGTKLDLK

SEQ ID NO: 70 - DNA, 321 bp, Mus musculus
  source 1..321: mol_type = other DNA
  CDS 1..321: protein_id = 69; translation = DIQVTQSPASLSASVGETVTITCRASGNIYSFLAWYQQKQGKSP QLLVYNAKTLAEGVPSRFSGSGSGTQFSLKISSLQPEDFGSYYCQHHYGIPLTFGAGT KLDLK
gacatccaggtgactcagtctccagcctccctatctgcgtctgtgggagaaactgtcaccatcacatgtcgcgcaagtgggaatatttacagttttttagcatggtatcagcagaaacagggaaaatctcctcaactcctggtctataatgcaaaaactttagcagaaggtgtgccatcaaggttcagtggcagtggatcaggcacacagttttctctgaagatcagcagcctgcagcctgaagattttgggagttattactgtcaacatcattatggtattcctctcacgttcggtgctgggaccaagttggacctgaaa

SEQ ID NO: 71 - protein, 10 aa, Mus musculus
GYTFASFGIS

SEQ ID NO: 72 - protein, 17 aa, Mus musculus
EIYPRSGTTYFNEKFRG

SEQ ID NO: 73 - protein, 12 aa, Mus musculus
AREGVRRYAMDY

SEQ ID NO: 74 - protein, 11 aa, Mus musculus
RASGNIYSFLA

SEQ ID NO: 75 - protein, 7 aa, Mus musculus
NAKTLAE

SEQ ID NO: 76 - protein, 9 aa, Mus musculus
QHHYGIPLT

SEQ ID NO: 77 - protein, 119 aa, Mus musculus
QFQLQQSGAELARPGASVKLSCKASGYTFASFGISWVKQRTGQGLEYIGEIYPRSGTTYFNEKFRGKATLTADKSSSTAYMELRSLTSEDSAVYFCAREGVRRYAMDYWGQGTSVTVSS

SEQ ID NO: 78 - DNA, 357 bp, Mus musculus
  source 1..357: mol_type = other DNA
  CDS 1..357: protein_id = 77; translation = QFQLQQSGAELARPGASVKLSCKASGYTFASFGISWVKQRTGQG LEYIGEIYPRSGTTYFNEKFRGKATLTADKSSSTAYMELRSLTSEDSAVYFCAREGVR RYAMDYWGQGTSVTVSS
cagtttcagctgcagcagtctggagctgagctggcgaggcctggggcttcagtgaagctgtcctgcaaggcttctggctacaccttcgcaagctttggtataagctgggtgaaacagagaactggacagggccttgaatacattggagagatttatcctagaagtggtactacttacttcaatgagaaattcaggggcaaggccacactgactgcagacaaatcatccagcacagcgtacatggagctccgcagcctgacatctgaggactctgcggtctatttctgtgcaagagagggggtacgacggtatgctatggactactggggtcaaggaacctcagtcaccgtctcctca

SEQ ID NO: 79 - protein, 107 aa, Mus musculus
DIQVTQSPASLSASVGETVTITCRASGNIYSFLAWYQQKQGKSPQLLVYNAKTLAEGVPSRFSGSGSGTQFSLKISSLQPEDFGSYYCQHHYGIPLTFGGGTKLDLK

SEQ ID NO: 80 - DNA, 321 bp, Mus musculus
  source 1..321: mol_type = other DNA
  CDS 1..321: protein_id = 79; translation = DIQVTQSPASLSASVGETVTITCRASGNIYSFLAWYQQKQGKSP QLLVYNAKTLAEGVPSRFSGSGSGTQFSLKISSLQPEDFGSYYCQHHYGIPLTFGGGT KLDLK
gacatccaggtgactcagtctccagcctccctatctgcgtctgtgggagaaactgtcaccatcacatgtcgcgcaagtgggaatatttacagttttttagcatggtatcagcagaaacagggaaaatctcctcagctcctggtctataatgcaaaaaccttagcagaaggtgtgccatcaaggttcagtggcagtggatcaggcacacagttttctctgaagattagcagcctgcagcctgaagattttgggagttattactgtcaacatcattatggtattcctctcacgttcggtggtgggaccaagttggacctgaaa

SEQ ID NO: 81 - protein, 10 aa, Mus musculus
GYTFASFGIS

SEQ ID NO: 82 - protein, 17 aa, Mus musculus
EIYPRSGTTYFNEKFRG

SEQ ID NO: 83 - protein, 12 aa, Mus musculus
AREGVRRYAMDY

SEQ ID NO: 84 - protein, 11 aa, Mus musculus
RASGNIYSFLA

SEQ ID NO: 85 - protein, 7 aa, Mus musculus
NAKTLAE

SEQ ID NO: 86 - protein, 9 aa, Mus musculus
QHHYGIPLT

SEQ ID NO: 87 - protein, 119 aa, Mus musculus
QLQLQQSGAELARPGASVKLSCKASGYTFASFGISWVKQRAGQGLEYIGEIYPRSGTTYFNEKFRGKATLTADKSSSTAYMELRRLTSEDSAVYFCAREGVRRYAMDYWGQGTSVTVSS

SEQ ID NO: 88 - DNA, 357 bp, Mus musculus
  source 1..357: mol_type = other DNA
  CDS 1..357: protein_id = 87; translation = QLQLQQSGAELARPGASVKLSCKASGYTFASFGISWVKQRAGQG LEYIGEIYPRSGTTYFNEKFRGKATLTADKSSSTAYMELRRLTSEDSAVYFCAREGVR RYAMDYWGQGTSVTVSS
cagcttcagctgcagcagtctggagctgagctggcgaggcctggggcttcagtgaagctgtcctgcaaggcttctggctacaccttcgcaagttttggtataagttgggtgaagcagagagctggacagggccttgagtacattggagagatttatcctagaagtggtactacttacttcaatgagaagttcaggggcaaggccacactgactgcagacaaatcatccagcacagcgtacatggagctccgcagactgacatctgaggactctgcggtctatttctgtgcaagagagggggtacgacggtatgctatggactactggggtcaaggaacctcagtcaccgtctcctca

SEQ ID NO: 89 - protein, 107 aa, Mus musculus
DIQVTQSPASLSASVGETVTITCRASGNIYSFLAWYQQKQGKSPQLLVYNAKTLAEGVPSRFSGSGSGTQFSLKINSLQPEDFGSYYCQHHYGIPLTFGAGTKLDLK

SEQ ID NO: 90 - DNA, 321 bp, Mus musculus
  source 1..321: mol_type = other DNA
  CDS 1..321: protein_id = 89; translation = DIQVTQSPASLSASVGETVTITCRASGNIYSFLAWYQQKQGKSP QLLVYNAKTLAEGVPSRFSGSGSGTQFSLKINSLQPEDFGSYYCQHHYGIPLTFGAGT KLDLK
gacatccaggtgactcagtctccagcctccctatctgcgtctgtgggagaaactgtcaccatcacatgtcgcgcaagtgggaatatttacagttttttagcatggtatcagcagaaacagggaaaatctcctcagctcctggtctataatgcaaaaaccttagcagaaggtgtgccatcaaggttcagtggcagtggatcaggcacacagttttctctgaagatcaacagcctgcagcctgaagattttgggagttattactgtcaacatcattatggtattcctctcacgttcggtgctgggaccaagttggacctgaaa

SEQ ID NO: 91 - protein, 10 aa, Mus musculus
GYTFTSFGVN

SEQ ID NO: 92 - protein, 17 aa, Mus musculus
QIYPRTGTTYHNERFKG

SEQ ID NO: 93 - protein, 11 aa, Mus musculus
AREGDRYSLAY

SEQ ID NO: 94 - protein, 11 aa, Mus musculus
RASENIYRFLA

SEQ ID NO: 95 - protein, 7 aa, Mus musculus
NAKTLVE

SEQ ID NO: 96 - protein, 9 aa, Mus musculus
QHHYGIPLT

SEQ ID NO: 97 - protein, 118 aa, Mus musculus
QVQVQQSGAELARPGASVRLSCKASGYTFTSFGVNWVKQRTGQGLEWIGQIYPRTGTTYHNERFKGKATLTADKSSSTAYMELRSLTSEDSAVYFCAREGDRYSLAYWGQGTLVTVSA

SEQ ID NO: 98 - DNA, 354 bp, Mus musculus
  source 1..354: mol_type = other DNA
  CDS 1..354: protein_id = 97; translation = QVQVQQSGAELARPGASVRLSCKASGYTFTSFGVNWVKQRTGQG LEWIGQIYPRTGTTYHNERFKGKATLTADKSSSTAYMELRSLTSEDSAVYFCAREGDR YSLAYWGQGTLVTVSA
caggttcaggtacagcagtccggagctgagctggcgaggcctggggcatcagtgaggctgtcctgcaaggcttctggctacaccttcacaagttttggtgtaaactgggtgaagcagagaactggacagggccttgagtggattggacagatttatcctagaactggtactacttaccacaatgagaggttcaagggcaaggccacactgactgcagacaagtcctccagcacagcgtacatggagctccgcagcctgacatctgaggactctgcggtctatttctgtgcacgagagggagataggtactcacttgcttactggggccaagggactctggtcactgtctctgca

SEQ ID NO: 99 - protein, 107 aa, Mus musculus; identical to SEQ ID NO: 59

SEQ ID NO: 100 - DNA, 321 bp, Mus musculus; identical to SEQ ID NO: 60
  source 1..321: mol_type = other DNA
  CDS 1..321: protein_id = 99; translation = DIQMTQSPASLSASVGETVTITCRASENIYRFLAWYQQKQGKSP QLLVYNAKTLVEGVPSRFSGSGSGTQFSLKINNLQPEDFGSYYCQHHYGIPLTFGAGT KLELK

SEQ ID NO: 101 - protein, 10 aa, Mus musculus
GYTFTSFGVN

SEQ ID NO: 102 - protein, 17 aa, Mus musculus
QIYPRSGTTYHNERFKG

SEQ ID NO: 103 - protein, 11 aa, Mus musculus
AREGDRYSLAY

SEQ ID NO: 104 - protein, 11 aa, Mus musculus
RASENIYRFLA

SEQ ID NO: 105 - protein, 7 aa, Mus musculus
NAKTLAE

SEQ ID NO: 106 - protein, 9 aa, Mus musculus
QHHYGIPLT

SEQ ID NO: 107 - protein, 118 aa, Mus musculus
QVQVHQSGAELARPGASVRLSCKASGYTFTSFGVNWVKQRTGQGLEWIGQIYPRSGTTYHNERFKGKATLTADKSSSTAYMELRSLTSEDSAVYFCAREGDRYSLAYWGQGTLVTVSA

SEQ ID NO: 108 - DNA, 354 bp, Mus musculus
  source 1..354: mol_type = other DNA
  CDS 1..354: protein_id = 107; translation = QVQVHQSGAELARPGASVRLSCKASGYTFTSFGVNWVKQRTGQG LEWIGQIYPRSGTTYHNERFKGKATLTADKSSSTAYMELRSLTSEDSAVYFCAREGDR YSLAYWGQGTLVTVSA
caggttcaggtacaccagtccggagctgagctggcgaggcctggggcatcagtgaggctgtcctgcaaggcttctggctacaccttcacgagctttggtgtaaactgggtgaagcagagaactggacagggccttgagtggattggacagatttatcctagaagtggtactacttaccacaatgagaggttcaagggcaaggccacactgactgcagacaaatcctccagcacagcgtacatggagctccgcagcctgacatctgaggactctgcggtctatttctgtgcaagagagggagataggtactcacttgcttactggggccaagggactctggtcactgtctctgca

SEQ ID NO: 109 - protein, 107 aa, Mus musculus
DIQMTQSPASLSASVGETVTITCRASENIYRFLAWYQQKQGKSPQLLVSNAKTLAEGVPSRFSGSGSGTQFSLKINYLQPEDFGNYYCQHHYGIPLTFGAGTKLELK

SEQ ID NO: 110 - DNA, 321 bp, Mus musculus
  source 1..321: mol_type = other DNA
  CDS 1..321: protein_id = 109; translation = DIQMTQSPASLSASVGETVTITCRASENIYRFLAWYQQKQGKSP QLLVSNAKTLAEGVPSRFSGSGSGTQFSLKINYLQPEDFGNYYCQHHYGIPLTFGAGT KLELK
gacatccagatgactcagtctccagcctccctatctgcatctgtgggagaaactgtcaccatcacatgtcgagcaagtgagaatatttacaggtttttagcatggtatcagcagaaacagggaaaatctcctcagctcctggtctctaatgcaaaaaccttagcagaaggtgtgccatcaaggttcagtggcagtggatcaggcacacagttttctctgaagatcaactacctacagcctgaagattttgggaattattactgtcaacatcattatggtattccgctcacgttcggtgctgggaccaagctggagctgaaa

SEQ ID NO: 111 - protein, 364 aa, Homo sapiens
MAAISTSIPVISQPQFTAMNEPQCFYNESIAFFYNRSGKHLATEWNTVSKLVMGLGITVCIFIMLANLLVMVAIYVNRRFHFPIYYLMANLAAADFFAGLAYFYLMFNTGPNTRRLTVSTWLLRQGLIDTSLTASVANLLAIAIERHITVFRMQLHTRMSNRRVVVVIVVIWTMAIVMGAIPSVGWNCICDIENCSNMAPLYSDSYLVFWAIFNLVTFVVMVVLYAHIFGYVRQRTMRMSRHSSGPRRNRDTMMSLLKTVVIVLGAFIICWTPGLVLLLLDVCCPQCDVLAYEKFFLLLAEFNSAMNPIIYSYRDKEMSATFRQILCCQRSENPTGPTEGSDRSASSLNHTILAGVHSNDHSVV

SEQ ID NO: 112 - protein, 364 aa, Mus musculus
MAAASTSSPVISQPQFTAMNEQQCFYNESIAFFYNRSGKYLATEWNTVSKLVMGLGITVCVFIMLANLLVMVAIYVNRRFHFPIYYLMANLAAADFFAGLAYFYLMFNTGPNTRRLTVSTWLLRQGLIDTSLTASVANLLAIAIERHITVFRMQLHTRMSNRRVVVVIVVIWTMAIVMGAIPSVGWNCICDIDHCSNMAPLYSDSYLVFWAIFNLVTFVVMVVLYAHIFGYVRQRTMRMSRHSSGPRRNRDTMMSLLKTVVIVLGAFIVCWTPGLVLLLLDVCCPQCDVLAYEKFFLLLAEFNSAMNPIIYSYRDKEMSATFRQILCCQRNENPNGPTEGSDRSASSLNHTILAGVHSNDHSVV

SEQ ID NO: 113 - DNA, 37 bp, synthetic construct
  source 1..37: mol_type = other DNA
gctagcgctaccggactcagatcccccccccccccdn

SEQ ID NO: 114 - DNA, 23 bp, synthetic construct
  source 1..23: mol_type = other DNA
accytgcatttgaactccttgcc

SEQ ID NO: 115 - DNA, 25 bp, synthetic construct
  source 1..25: mol_type = other DNA
actgccatcaatcttccacttgaca

SEQ ID NO: 116 - DNA, 44 bp, synthetic construct
  source 1..44: mol_type = other DNA
ggtttagtgaaccgtcagatccgctagcgctaccggactcagat

SEQ ID NO: 117 - DNA, 23 bp, synthetic construct
  source 1..23: mol_type = other DNA
ctggacagggatccagagttcca

SEQ ID NO: 118 - DNA, 25 bp, synthetic construct
  source 1..25: mol_type = other DNA
actgaggcacctccagatgttaact

SEQ ID NO: 119 - protein, 351 aa, Homo sapiens
MVIMGQCYYNETIGFFYNNSGKELSSHWRPKDVVVVALGLTVSVLVLLTNLLVIAAIASNRRFHQPIYYLLGNLAAADLFAGVAYLFLMFHTGPRTARLSLEGWFLRQGLLDTSLTASVATLLAIAVERHRSVMAVQLHSRLPRGRVVMLIVGVWVAALGLGLLPAHSWHCLCALDRCSRMAPLLSRSYLAVWALSSLLVFLLMVAVYTRIFFYVRRRVQRMAEHVSCHPRYRETTLSLVKTVVIILGAFVVCWTPGQVVLLLDGLGCESCNVLAVEKYFLLLAEANSLVNAAVYSCRDAEMRRTFRRLLCCACLRQSTRESVHYTSSAQGGASTRIMLPENGHPLMDSTL

SEQ ID NO: 120 - protein, 348 aa, Mus musculus
MGQCYYNETIGFFYNNSGKELSLHWRPKDVVVVALGLTVSVLVLLTNLLVIAAIASNRRFHQPIYYLLGNLAAADLFAGMAYLFLMFHTGPRTARLSIKGWFLRQGLLDTSLTASVATLLAIAVERHRSVMAVQLHSRLPRGRVVTLIVGVWAAALGLGLLPAHFWHCLCDLDSCSRMVPLFSRSYLAAWALSSLLVFLLMVAVYTRIFFYVRRRVERMAEHVSCHPRYRETTLSLVKTVVIILGAFVVCWTPGQVVLLLDGLDCKSCNVLAVEKYFLLLAEANSLVNAVVYSCRDAEMRRTFRRLLCCMCLRWSSHKSARYSASAQTGASTRIMLPENGRPLMDSTL

SEQ ID NO: 121 - protein, 353 aa, Homo sapiens
MNECHYDKHMDFFYNRSNTDTVDDWTGTKLVIVLCVGTFFCLFIFFSNSLVIAAVIKNRKFHFPFYYLLANLAAADFFAGIAYVFLMFNTGPVSKTLTVNRWFLRQGLLDSSLTASLTNLLVIAVERHMSIMRMRVHSNLTKKRVTLLILLVWAIAIFMGAVPTLGWNCLCNISACSSLAPIYSRSYLVFWTVSNLMAFLIMVVVYLRIYVYVKRKTNVLSPHTSGSISRRRTPMKLMKTVMTVLGAFVVCWTPGLVVLLLDGLNCRQCGVQHVKRWFLLLALLNSVVNPIIYSYKDEDMYGTMKKMICCFSQENPERRPSRIPSTVLSRSDTGSQYIEDSISQGAVCNKSTS

SEQ ID NO: 122 - protein, 354 aa, Mus musculus
MNECHYDKRMDFFYNRSNTDTADEWTGTKLVIVLCVGTFFCLFIFFSNSLVIAAVITNRKFHFPFYYLLANLAAADFFAGIAYVFLMFNTGPVSKTLTVNRWFLRQGLLDTSLTASLANLLVIAVERHMSIMRMRVHSNLTKKRVTLLILLVWAIAIFMGAVPTLGWNCLCNISACSSLAPIYSRSYLIFWTVSNLLAFFIMVAVYVRIYMYVKRKTNVLSPHTSGSISRRRAPMKLMKTVMTVLGAFVVCWTPGLVVLLLDGLNCKQCNVQHVKRWFLLLALLNSVMNPIIYSYKDEDMYNTMRKMICCALQDSNTERRPSRNPSTIHSRSETGSQYLEDSISQGPVCNKNGS

SEQ ID NO: 123 - protein, 122 aa, synthetic construct
QVQLVQSGAEVKKPGASVKVSCKASGYTFTSYGISWVRQAPGQGLEWMGEIYPRSGNTYYNEKFKGRVTLTADKSTSTAYMELRSLTSEDTAVYFCARESISRRLGWNFDVWGQGTTVTVSS

SEQ ID NO: 124 - protein, 107 aa, synthetic construct
DIQMTQSPSSLSASVGDTVTITCRASENIYSFLAWYQQKPGKAPKLLIYNAKTLTEGVPSRFSGSGSGTHFSLTINSLQPEDFGIYYCQHHYGPPLTFGQGTKLEIK

SEQ ID NO: 125 - protein, 122 aa, synthetic construct
QVQLQESGPGLVKPSETLSLTCTASGYTFTSYGISWVRQTPGKGLEWIGEIYPRSGNTYYNEKFKGRATLSADKSKNQASLKLKSVTAADTAIYFCARESISRRLGWNFDVWGRGTLVTVSG

SEQ ID NO: 126 - protein, 107 aa, synthetic construct
DIQMTQTPSSLSASVGDRVTITCRASENIYSFLAWYQQKPGEAPKRVVYNAKTLTEGVPSRFSGSGSGTDFTLTISSLQPEDFATYYCQHHYGPPLTFGQGTKLEIK

SEQ ID NO: 127 - protein, 122 aa, synthetic construct
QVQLVQSGAEVKKPGASVKVSCKASGYTFTSYGISWVRQAPGQGLEWIGEIYPRSGNTYYNEKFKGRATLTADKSTSTAYMELRSLRSDDTAVYFCARESISRRLGWNFDVWGTGTTVTVSS

SEQ ID NO: 128 - protein, 107 aa, synthetic construct
DIQMTQSPSSLSASVGDRVTITCRASENIYSFLAWYQQKPGKAPKLVVYNAKTLTEGVPSRFSGSGSGTDFTLTISSLQPEDFATYYCQHHYGPPLTFGAGTKLELK

SEQ ID NO: 129 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 107 aa, Homo sapiens [sequence omitted]